(19) United States
(12) Patent Application Publication
(10) Pub. No.: US 2008/0076674 A1
(43) Pub. Date: Mar. 27, 2008

(54) Novel oligonucleotide compositions and probe sequences useful for detection and analysis of non coding RNAs associated with cancer

(76) Inventors: Thomas Litman, Vaerlose (DK); Soren Moller, Holte (DK); Soren Morgenthaler Echwald, Humlebaek (DK)

Correspondence Address: CLARK & ELBING LLP, 101 FEDERAL STREET, BOSTON, MA, 02110, US

(21) Appl. No.: 11/825,667

(22) Filed: Jul. 6, 2007

(30) Foreign Application Priority Data
Jul. 6, 2006 (DK) PA2006/00929

(51) Int. Cl. C40B 30/04; C07H 21/00; C40B 40/06; C12Q 1/68

(52) U.S. Cl. 506/9; 128/898; 435/6; 506/16; 536/24.31

(57) ABSTRACT

The invention relates relates to ribonucleic acids and oligonucleotide probes useful for detection and analysis of non-coding RNAs, such as microRNAs and small nuclear RNA (snRNA), in particular small nucleolar RNAs (snoRNAs), and their precursors which are associated with cancer, and which can be used for characterising breast cancers or suspected cancer.

The present invention relates to methods for detection and analysis of noncoding RNAs associated with cancer. The invention furthermore relates to collections of oligonucleotide probes for detection and analysis of non-coding RNAs associated with cancer.

### BACKGROUND OF THE INVENTION

The present invention relates to the detection and analysis of target nucleotide sequences associated with cancer, such as breast cancer, more specifically to the methods employing the use of oligonucleotide probes that are useful for detecting and analyzing target nucleotide sequences associated with cancer, such as breast cancer, especially non-coding RNA target sequences associated with cancer, such as breast cancer, such as microRNAs (miRNAs), piRNAs, snRNAs and siRNAs sequences of interest, and precursors of such non-coding RNAs, for detecting differences between nucleic acid samples (e.g., such as samples from a breast cancer patient and a healthy patient or a tumor sample and a non tumorous sample from the same patient).

According to the World Health Organisation (WHO) more than 11 million people worldwide are diagnosed with cancer every year, and it is estimated that there will be 16 million new cases every year by 2020. Cancer causes 7 million deaths every year—or 12.5% of deaths worldwide. Furthermore, cancer is a complex disease affecting nearly every tissue in the body, and the conquest of cancer continues to pose great challenges to medical science. In fact, the age-adjusted mortality rate for cancer is about the same in the 21st century as it was 50 years ago!

Thus, there is an obvious medical need for better patient care through linking of cancer diagnosis and treatment, in order to fulfill the promises of personalized medicine.

By understanding the genetic and biochemical mechanisms by which cancers arise, through a characterization of cancer in molecular terms, physicians can improve the ways cancers are detected, classified, monitored and treated.

The first success story of linking molecular diagnostics and targeted cancer therapy is treatment of HER-2 positive breast cancer with the anti-HER-2 antibody Herceptin (trastuzumab; Genentec). This breast cancer treatment originally provided only modest benefits and some troubling side effects, for a broad patient population. However, once patients who expressed the HER2/neu gene were singled out the drugs efficacy shot up justifying the adverse events. Other cases of linking molecular diagnostics to therapy are Gleevec for CML and Tamoxifen anti-hormone therapy for ER/PR positive breast cancers.

However, targeting a single molecule is unlikely to result in a profound response or durable remission in all cancer patients. As our understanding of cancer advances it has become clear that cancer pathogenesis is the result of multiple molecules or systems gone awry.

Therefore, the “omic” technology—because of its ability to identify abnormal patterns of expression associated with cancers—is a promising approach to evaluate the heterogeneity of cancer patients. In response to the opportunities several companies have begun developing molecular cancer diagnostics based on proteomic, genomic as well as transcriptomic technologies. This trend signifies commercial validation of the molecular cancer diagnostic market.

MicroRNAs (miRNAs) have rapidly emerged as an important class of short endogenous RNAs that act as post-transcriptional regulators of gene expression by base-pairing with their target mRNAs. The 19-25 nucleotide (nt) mature miRNAs are processed sequentially from longer hairpin transcripts by the RNAse III ribonucleases Drosha (Lee, Y., et al., 2003. Nature 425: 415-419.) and Dicer (Hutvagner, G., et al., 2001. Science 293: 834-838, Ketting, R. F., et al., 2001. Genes Dev. 15: 2654-2659.). To date 4584 microRNAs have been annotated in vertebrates, invertebrates and plants according to the miRBase database release 9.2 in May 2007 (Griffiths-Jones, S. 2004. NAR 32 (Database issue), D109-D111), and many miRNAs that correspond to putative genes have also been identified. Some miRNAs have multiple loci in the genome (Reinhart, B. J., et al., 2002. Genes Dev. 16, 1616-1626.) and occasionally, several miRNA genes are arranged in tandem clusters (Lagos-Quintana, M., et al., 2001. Science 294: 853-858.). Recent bioinformatic predictions combined with array analyses, small RNA cloning and Northern blot validation indicate that the total number of miRNAs in vertebrate genomes is significantly higher than previously estimated and maybe as many as 1000 (Bentwich, I., et al., 2005. Nat. Genet. 37: 766-770, Berezikov, E., et al., 2005. Cell 120: 21-24, Xie, X., Lu, J., et al., 2005. Nature 434: 338-345.).

The first miRNAs genes to be discovered, lin-4 and let-7, base-pair incompletely to repeated elements in the 3′ untranslated regions (UTRs) of other heterochronic genes, and control developmental timing in the roundworm C. elegans by regulating translation directly and negatively via antisense RNA-RNA interaction (Lee, R. C., et al., 1993. Cell 75: 843-854., Reinhart, B. J., et al., 2000. Nature 403: 901-906.). The majority of plant miRNAs have perfect or near-perfect complementarity with their target sites and direct RISC-mediated target mRNA cleavage, whereas most animal miRNAs recognize their target sites located in 3′-UTRs by incomplete base-pairing, resulting in translational repression of the target genes (Bartel, D. P. 2004. Cell 116: 281-297.).

An increasing body of research shows that animal miRNAs play fundamental biological roles in cell growth and apoptosis (Brennecke, J., et al., 2003. Cell 113: 25-36.), hematopoietic lineage differentiation (Chen, C. Z., et al., 2004. Science 303: 83-86.), homeobox gene regulation (Yekta, S., et al., 2004. Science 304: 594-596.), neuronal asymmetry (Johnston, R. J. and Hobert, O. 2003. Nature 426: 845-849.), insulin secretion (Poy, M. N., et al., 2004. Nature 432, 226-230.), brain morphogenesis (Giraldez, A. J., et al., 2005. Science 308: 833-838.), cardiogenesis (Zhao, Y., et al., 2005. Nature 436: 214-220.) and late embryonic development in vertebrates (Chen, P. Y., et al., 2005. Genes Dev. 19: 1288-1293., Wienholds, E., et al., 2005. Science 309: 310-311.). Several studies have identified subclasses of miRNAs directly implicated in the regulation of mammalian brain development and neuronal differentiation (Krichevsky, A. M., et al., 2003. RNA 9: 1274-1281., Miska, E. A., et al., 2004. Genome Biology 5:R68., Sempere, L. F., et al., 2004. Genome Biol. 5: R13., Smirnova, L., et al., 2005. Eur J Neurosci. 21: 1469-77.). Interestingly, many neural miRNAs appear to be temporally regulated in cortical cultures copurifying with polyribosomes, suggesting that they may control localized translation of dendrite-specific mRNAs (Kim, J., et al., 2004. PNAS 101: 360-5.). The number of regulatory mRNA targets of vertebrate miRNAs was recently estimated by identifying conserved complementarity to the seed sequence of the miRNAs, suggesting that ˜30% of the human genes may be controlled by miRNAs, with an average of ˜200 mRNA targets per miRNA (Krek, A., et al., 2005. Nat. Genet. 37: 495-500., Lewis, B. P., et al., 2005. Cell 120: 15-20.).

The expanding inventory of human miRNAs along with their highly diverse expression patterns and high number of potential target mRNAs suggest that miRNAs are involved in a wide variety of human diseases. One is spinal muscular atrophy, a pediatric neurodegenerative disease caused by reduced protein levels or loss-of-function mutations of the survival of motor neurons gene (Paushkin, S., et al., 2002. Curr. Opin. Cell Biol. 14: 305-312.). Other diseases in which miRNAs or their processing machinery have been implicated, include fragile X mental retardation caused by absence of the fragile X mental retardation protein (Nelson, P., et al., 2003. TIBS 28: 534-540) and DiGeorge syndrome (Landthaler, M., et al., 2004. Curr. Biol. 14: 2162-2167.). In addition, perturbed miRNA expression patterns have been reported in many human cancers. For example, the human miRNA genes miR15a and miR16-1 are deleted or down-regulated in the majority of B-cell chronic lymphocytic leukemia cases, while more than 50% of the human miRNA genes are located in cancer-associated genomic regions or at fragile sites (Calin, G. A., et al. 2004. PNAS 101: 11755-11760.).

In a series of publications during recent years, it has become clear that microRNAs are extensively involved in cancer pathogenesis, and microRNAs have been shown to be differentially expressed in a number of cancers (Breast cancer: Iorio et al Cancer Res 2005; 65: 7065. Lung cancer: Yanaihara et al Cell Science 2006; 9: 189-198. Chronic lymphocytic leukaemia (CLL): Galin et al PNAS, 2004 101(32):11755-11760. Colon cancer: Cummins et al PNAS 2006, 103 (10):3687-3692. Prostate cancer: Volinia et al PNAS 2006; 103: 2257). In fact, in a landmark paper Lu et al (Nature 2005; 435:834-838) demonstrated differential expression of microRNAs in multiple cancers types, and that signatures based on approximately 200 microRNAs improve classification of poorly differentiated cancers over mRNA profiles.

Furthermore, the expected complexity of the “microRNA'nome” is far smaller than the human transcriptome with the total number of microRNAs being approximately limited to between 800 to 1000. Therefore, a microRNA cancer signature can be predicted to include from 5-20 microRNAs, suggesting that microRNA based theranostics will be of limited complexity and far more robust than mRNA profiles.

Taken together microRNAs constitute a new class of non-coding RNAs that plays a significant role in determining gene expression, microRNAs are differentially expressed in human cancers, and a series of recent publications show that microRNAs classify human cancers; in some cases improvement over mRNA classification is observed.

Breast cancer is one of the most prevalent cancer forms with 212,920 newly diagnosed cases in US (predicted for 2006) and approximately 370,100 in EU (actual cases in 2004). Furthermore, it is estimated that worldwide breast cancer affects ˜1 million women annually.

The primary treatment for breast cancer is surgery followed—in many cases—by radiation. Tumors are classified based on the TNM system that relays on histology of the primary tumor (T), regional lymph nodes (N), as well as distant metastasis (M). It should be noted that US staging system and the EU (St. Gallen) criteria for breast cancer classification differ slightly.

The adjuvant therapy chosen to follow surgery is selected on the basis of multiple factors such as Estrogen-receptor (ER) and Progesterone-receptor (PR) protein status and additional pathologic characteristics, including tumor grade (based on TMN classification), proliferative activity, human epidermal growth factor receptor 2 (HER2/neu) status, menopausal status, as well as the general health of the patient. The strongest predictors for risk of metastasis are lymph node status and histological grade.

Depending on disease classification (staging) patients receive a mixture of radiation, anti-hormone therapy (Tamoxifen or Aromatase inhibitors) and chemotherapy. The chemotherapy may be selected from a series of different treatment regiments such as CMF (cyclophosphamide, methotrexate and 5-FU) or FAC (Cyclophosphamide, adriamycin, and 5-FU).

The current classification is not adequate, because breast cancer patients with the same stage of disease can exhibit very different response to treatment as well as overall outcome. Chemotherapy and/or hormonal therapy reduces the risk of distant metastases by one-third; however, 70-80% of patients receiving this treatment would have survived without it, and therefore more accurate prognostic methods are needed to improve the selection of patients for adjuvant systemic therapy.

The present invention allows for the determination of microRNA signatures that improve the classification of early diagnosed cancers, such as breast cancers. The microRNA signatures—following form the role of microRNAs in cancer—reveal the true cancerous potential of the tumor, and enable physicians to select the appropriate treatment. microRNA based cancer, such as breast cancer, classification may significantly benefit patient care, because recurrence rate may be improved due to adequate treatment of traditionally classified low risk patients, and suitable therapy, such as adjuvant chemotherapy may be deselected for the large group of patients that do not benefit from it.

PCT/DK2005/000838, and U.S. application Ser. No. 11/324,177, both hereby incorporated by reference, disclose methods for the detection of microRNAs (miRNAs) using oligonucleotides which comprise nucleotide analogues, such as locked nucletic acids (LNAs).

WO2005/098029, hereby incorporated by reference, discloses a method using oligonucleotides for the detection, quantification, monitoring of expression of siRNA and/or miRNA. It is suggested that the method can be used for determining the differences between nucleic acid samples from e.g. a cancer patient.

The Sanger Institute publishes known miRNA sequences in the miRBASE database (http://microrna.sanger.ac.uk/sequences/index.shtml). To date there are 475 human miRNAs present in the miRBASE database. WO2006/015312 discloses sets of genetic markers which can be correlated with a prognosis of breast cancer.

Lau et al., Science. Jun. 15, 2006 Girard et al., Nature. Jun. 4, 2006 Aravin et al., Nature. Jun. 4, 2006 Grivna et al., Genes Dev. Jun. 9, 2006 disclose piRNAs, which are non-coding RNAs of up to 30 bases in length which are expressed in the gonads. piRNAs interact with Piwi, which is an Arganaut like protein.

Iorio et al, (Cancer Res 2005; 65 (16), pp 7065-7070 discloses miRNAs whose expression profile is altered between breast cancer and non tumor cells.

### SUMMARY OF THE INVENTION

The invention provides for a method for the characterisation of cancer, in a sample derived or obtained from a mammal, preferably a human being, said method comprising the following steps:
a. obtaining at least one test sample, such as a biopsy sample, of a tumor or of a putative tumor, from a patient;
b. presenting a first population of nucleic acid molecules, prepared from said at least one test sample. wherein said first population comprises non-coding RNAs;
c. hybridizing said first population of target molecules, against at least one first detection probe, wherein said at least one first detection probe comprises a recognition sequence derived from a non-coding RNA or precursor thereof;
d. detecting a signal emitted during or subsequent to said hybridization step, said signal providing data which is indicative of hybridization of said at least one first detection probe to a first a non-coding RNA or precursor thereof present within said first population of target molecules;
e. comparing said signal data obtained to reference data, which optionally maybe obtained from said control sample, to provide characterisation of at least one feature of said cancer.

The invention provides for a method for the characterisation of cancer, in a sample derived or obtained from a mammal, preferably a human being, said method comprising the following steps:
a. Obtaining at least one test sample, such as a biopsy sample, of a tumor or of a putative tumor, from a patient;
b. Presenting a first population of nucleic acid molecules, prepared from said at least one test sample, wherein said first population comprises small nucleolar RNA or miRNA;
c. Hybridizing said first population of target molecules, against at least one first detection probe, wherein said at least one first detection probe comprises recognition sequence derived from a small nuclear RNA (snRNA) or miRNA or precursor thereof;
d. Detecting a signal emitted during or subsequent to said hybridization step, said signal providing data which is indicative of hybridization of said at least one first detection probe to a first a small nuclear RNA (snRNA) or miRNA or precursor thereof present within said first population of target molecules;
e. Comparing said signal data obtained to reference data, which optionally may be obtained from said control sample, to provide characterisation of at least one feature of said cancer.

The invention further provides for the use of at least one detection probe which comprises a recognition sequence which is complementary to a small nuclear RNA (snRNA) or miRNA precursor thereof for the characterisation of cancer.

The invention further provides for a collection of detection probes, wherein each member of said collection comprises a recognition sequence consisting of nucleobases and/or affinity enhancing nucleobase analogues, wherein said collection of detection probes comprises at least one detection probe which is complementary to a small nuclear RNA (snRNA) or miRNA or precursor thereof.

The invention further provides for a kit for the detection of cancer, said kit comprising at least one detection probe which is complementary to a small nuclear RNA (snRNA) or miRNA or precursor thereof.

The invention further provides for a method of for the treatment of cancer, said method comprising
a. Isolating at least one tissue sample from a patient suffering from cancer;
b. Performing the method for the characterisation of cancer according to the invention, to identify at least one feature of said cancer;
c. Based on at least one feature identified in step b) diagnosing the physiological status of the cancer disease in said patient;
d. Selecting an appropriate form of therapy for said patient based on the said diagnosis;
e. Administering said appropriate form of therapy.

The invention further provides for a method for the determination of suitability of a cancer patient for treatment comprising:
a. Isolating at least one tissue sample from a patient suffering from cancer;
b. Performing the method for the characterisation of cancer according to the invention, to identify at least one feature of said cancer;
c. Based on the at least one feature identified in step b) diagnosing the physiological status of the patient;
d. Based on the said diagnosis obtained in step c) determining whether said patient would benefit from treatment of said cancer.

The invention further provides for a method for the determination of the origin of a metastatic cancer, or a cancer suspected of being a metastasis, comprising:
a. Isolating at least one tissue sample of a metastatic cancer, or a cancer suspected of being a metastasis, from a patient;
b. Performing the method for the characterisation of cancer according to the invention, to identify the origin of said metastatic cancer.

The invention further provides for a method for the determination of the likely prognosis of a cancer patient comprising:
a. Isolating at least one tissue sample from a patient suffering from cancer;
b. Performing the method for the characterisation of cancer according to the invention, to identify at least one feature of said cancer;
c. wherein said feature allows for the determination of the likely prognosis of said cancer patient.

The invention further provides for a method for specific isolation, purification, amplification, detection, identification, quantification, inhibition or capture of a target nucleotide sequence in a sample from a cancer, said method comprising contacting said sample with a detection probe as which is complementary to a snRNA or miRNA under conditions that facilitate hybridization between said member/probe and said snRNA or miRNA sequence.

### BRIEF DESCRIPTION OF THE FIGURES

FIG. 1. M-A plot showing all miRNA signals before averaging

FIG. 2. The miRNAs that were reported as down-regulated in breast cancer by Iorio et al. were confirmed, and in 7 out of 8 cases with higher contrast between normal and cancer

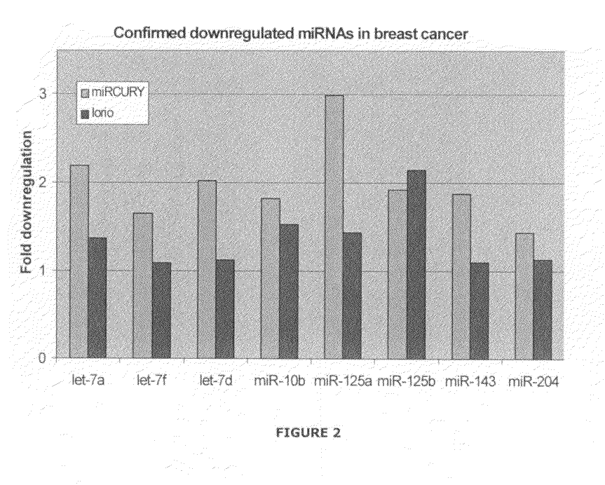

FIG. 3. Most of the miRNAs that were reported as up-regulated by Iorio et al, were also detected as up-regulated with the miRCURY microarray. In particular, miR-21 was highly expressed in breast cancer tissue compared to normal adjacent tissue.

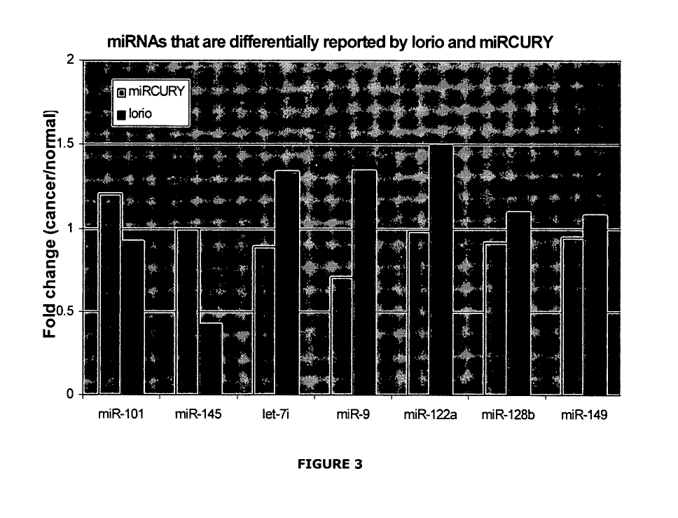

FIG. 4. For these miRNAs, our findings contrast those of Iorio et al. This discrepancy could be due to 1) low signal, where ratios become unreliable, 2) the wide range of miRNA expression reported by Iorio et al (e.g. miR-145: 1.65-14.56 for normal breast, and 0.92-8.46 for breast cancer), and 3) our limited sample material.

FIG. 5. Dilution series for the human miR-145 real-time quantitative PCR assay.

FIG. 6. Quantitative RT-PCR data for selected miRNAs and U6 snoRNA.

### DETAILED DESCRIPTION OF THE INVENTION

The invention provides for a method for the characterisation of cancer, in a sample derived or obtained from a mammal, preferably a human being, said method comprising the following steps:
a. Obtaining at least one test sample, such as a biopsy sample, of a tumor or of a putative tumor, from a patient, and optionally at least one control sample;
b. Presenting a first population of nucleic acid molecules, prepared from said at least one test sample, and optionally a second population of nucleic acid molecules, prepared from said control sample;
c. Hybridizing said first population of target molecules, and optionally said second population of target molecules, against at least one detection probe, wherein said at least one detection probe comprises a recognition sequence derived from a non-coding RNA sequence associated with said cancer, such as a non-coding RNA sequence selected from the group consisting of microRNA (miRNA), siRNA piRNA, and snRNA, and precursor sequences thereof;
d. Detecting a signal emitted during or subsequent to said hybridization step, said signal providing data which is indicative of hybridization of said at least one detection probe to a first complementary target within said first population of target molecules;
e. Comparing said signal data obtained to reference data, which optionally maybe obtained from said control sample, to provide characterisation of at least one feature of said cancer.

The invention also provides for the use of at least one detection probe which is capable of hybridizing to a non-coding RNA target, such as a microRNA (miRNA), siRNA, piRNA or snRNA, for the characterisation of cancer, wherein said detection probe hybridizes to at least one non coding RNA associated with cancer.

The invention also provides for a collection of detection probes, wherein each member of said collection comprises a recognition sequence consisting of nucleobases and/or affinity enhancing nucleobase analogues, wherein said collection of detection probes comprises at least one member which is selected for its ability to hybridize to one or more non-ncoding RNAs which are associated with cancer, wherein said one or more non-ncoding RNAs are as defined herein.

The invention also provides for a kit for the detection of cancer, said kit comprising at least one detection probe (and/or at least one detection probe pair) according to the invention, wherein said detection probe hybridizes to at least one non-coding RNA associated with cancer.

The invention also provides for pairs of detection probes, wherein said detection probe pair comprise of a first detection probe which is capable of hybridizing to a further complementary target, such as a precursor non-coding RNA, and a second detection probe which is capable of hybridizing to said first complementary target, such as the corresponding mature non-coding RNA.

The invention also provides for a method for the treatment of cancer, said method comprising
a. Isolating at least one tissue sample from a patient suffering from cancer;
b. Performing the characterisation of the at least one tissue sample according to the method of characterisation of cancer according to the invention and/or by use of the collection of detection probes or kit according to the invention;
c. Based on the at least one feature identified in step b) diagnosing the physiological status of the cancer disease in said patient;
d. Selecting an appropriate form of therapy for said patient based on the said diagnosis;
e. Administering said appropriate form of therapy.

The invention also provides for a method for the determination of suitability of a cancer patient for treatment comprising:
a. Isolating at least one tissue sample from a patient suffering from cancer;
b. Performing the characterisation of the at least one tissue sample according to the method of characterisation of cancer according to the invention and/or by use of the collection of detection probes or kit according to the invention;
c. Based on the at least one feature identified in step b) diagnosing the physiological status of the patient;
d. Based on the said diagnosis obtained in step c) determining whether said patient would benefit from treatment of said cancer.

The invention also provides for a method for the determination of the origin of a metastatic cancer, or a cancer suspected of being a metastatic cancer, comprising:
a. Isolating at least one tissue sample from a patient suffering from cancer, or suspected of having cancer, such as cancer, or a metastatic cancer, or suspected metastatic cancer, which may have originated from a cancer tumor;
b. Performing the characterisation of the at least one tissue sample according to the method of characterisation of cancer according to the invention and/or by use of the collection of detection probes or kit according to the invention.
wherein said feature allows the identification of the origin of said metastatic cancer to be determined.

The invention also provides for a method for the determination of the likely prognosis of a cancer patient comprising:
a. Isolating at least one tissue sample from a patient suffering from cancer;
b. Performing the characterisation of the at least one tissue sample according to the method of characterisation of cancer according to the invention and/or the use of the collection of detection probes or kit according to the invention.
to identify at least one feature of said cancer wherein said feature allows for the determination of the likely prognosis of said cancer patient.

The invention also provides for a method for specific isolation, purification, amplification, detection, identification, quantification, inhibition or capture of a target nucleotide sequence in a sample, said method comprising contacting said sample with a detection probe according to the invention under conditions that facilitate hybridization between said member/probe and said target nucleotide sequence, wherein said target nucleotide sequence is, or is derived from a non-coding RNA associated with cancer.

The invention also provides for new molecular markers for cancer, and the use of such markers in the methods according to the invention, and for use in the collection of probes and/or kits according to the invention.

In another aspect the invention features detection probe sequences containing a ligand, which said ligand means something, which binds. Such ligand-containing detection probes of the invention are useful for isolating and/or detection target RNA molecules from complex nucleic acid mixtures, such as miRNAs, their cognate target mRNAs, siRNAs, piRNAs and snRNAs.

The invention therefore also provides for detection probes, such as oligonucleotide compositions, which are ligands to the molecular markers according to the invention.

In another aspect the invention features detection probes whose sequences have been furthermore modified by Selectively Binding Complementary (SBC) nucleobases, i.e. modified nucleobases that can make stable hydrogen bonds to their complementary nucleobases, but are unable to make stable hydrogen bonds to other SBC nucleobases. Such SBC monomer substitutions are especially useful when highly self-complementary detection probe sequences are employed. As an example, the SBC nucleobase A′, can make a stable hydrogen bonded pair with its complementary unmodified nucleobase, T. Likewise, the SBC nucleobase T′ can make a stable hydrogen bonded pair with its complementary unmodified nucleobase, A. However, the SBC nucleobases A′ and T′ will form an unstable hydrogen bonded pair as compared to the base pairs A′-T and A-T′. Likewise, a SBC nucleobase of C is designated C′ and can make a stable hydrogen bonded pair with its complementary unmodified nucleobase G, and a SBC nucleobase of G is designated G′ and can make a stable hydrogen bonded pair with its complementary unmodified nucleobase C, yet C′ and G′ will form an unstable hydrogen bonded pair as compared to the base pairs C′-G and C-G′. A stable hydrogen bonded pair is obtained when 2 or more hydrogen bonds are formed e.g. the pair between A′ and T, A and T′, C and G′, and C′ and G. An unstable hydrogen bonded pair is obtained when 1 or no hydrogen bonds is formed e.g. the pair between A′ and T′, and C′ and G′. Especially interesting SBC nucleobases are 2,6-diaminopurine (A′, also called D) together with 2-thio-uracil (U′, also called 2SU)(2-thio-4-oxo-pyrimidine) and 2-thio-thymine (T′, also called 2ST)(2-thio-4-oxo-5-methyl-pyrimidine).

In another aspect the detection probe sequences of the invention are covalently bonded to a solid support by reaction of a nucleoside phosphoramidite with an activated solid support, and subsequent reaction of a nucleoside phosphoramide with an activated nucleotide or nucleic acid bound to the solid support. In some embodiments, the solid support or the detection probe sequences bound to the solid support are activated by illumination, a photogenerated acid, or electric current. In other embodiments the detection probe sequences contain a spacer, e.g. a randomized nucleotide sequence or a non-base sequence, such as hexaethylene glycol, between the reactive group and the recognition sequence. Such covalently bonded detection probe sequence populations are highly useful for large-scale detection and expression profiling of mature miRNAs, stem-loop precursor miRNAs, siRNAs, piRNAs, snRNAs and other non-coding RNAs.

The present oligonucleotide compositions and detection probe sequences of the invention are highly useful and applicable for detection of individual small RNA molecules in complex mixtures composed of hundreds of thousands of different nucleic acids, such as detecting mature miRNAs, their target mRNAs, piRNAs, snRNAs or siRNAs, by Northern blot analysis or for addressing the spatiotemporal expression patterns of miRNAs, siRNAs or other non-coding RNAs as well as mRNAs by in situ hybridization in whole-mount.

The oligonucleotide compositions and detection probe sequences are especially applicable for accurate, highly sensitive and specific detection and quantitation of microRNAs and other non-coding RNAS, which are useful as biomarkers for diagnostic purposes of human diseases, such as breast cancer, as well as for antisense-based intervention, targeted against tumorigenic miRNAs and other non-coding RNAs.

The detection probes, detection probe pairs, and oligonucleotide compositions and probe sequences which hybridize to the molecular markers according to the invention are furthermore applicable for sensitive and specific detection and quantitation of microRNAs, which can be used as biomarkers for the identification of the primary site of metastatic tumors of unknown origin.

### Definitions

For the purposes of the subsequent detailed description of the invention the following definitions are provided for specific terms, which are used in the disclosure of the present invention:

In the present context “ligand” means something, which binds. Ligands may comprise biotin and functional groups such as: aromatic groups (such as benzene, pyridine, naphtalene, anthracene, and phenanthrene), heteroaromatic groups (such as thiophene, furan, tetrahydrofuran, pyridine, dioxane, and pyrimidine), carboxylic acids, carboxylic acid esters, carboxylic acid halides, carboxylic acid azides, carboxylic acid hydrazides, sulfonic acids, sulfonic acid esters, sulfonic acid halides, semicarbazides, thiosemicarbazides, aldehydes, ketones, primary alcohols, secondary alcohols, tertiary alcohols, phenols, alkyl halides, thiols, disulphides, primary amines, secondary amines, tertiary amines, hydrazines, epoxides, maleimides, C₁-C₂₀ alkyl groups optionally interrupted or terminated with one or more heteroatoms such as oxygen atoms, nitrogen atoms, and/or sulphur atoms, optionally containing aromatic or mono/polyunsaturated hydrocarbons, polyoxyethylene such as polyethylene glycol, oligo/polyamides such as poly-β-alanine, polyglycine, polylysine, peptides, oligo/polysaccharides, oligo/polyphosphates, toxins, antibiotics, cell poisons, and steroids, and also “affinity ligands”, i.e. functional groups or biomolecules that have a specific affinity for sites on particular proteins, antibodies, poly- and oligosaccharides, and other biomolecules.

The singular form “a”, “an” and “the” include plural references unless the context clearly dictates otherwise. For example, the term “a cell” includes a plurality of cells, including mixtures thereof. The term “a nucleic acid molecule” includes a plurality of nucleic acid molecules.

“Transcriptome” refers to the complete collection of transcriptional units of the genome of any species. In addition to protein-coding mRNAs, it also represents non-coding RNAs, such as small nucleolar RNAs, siRNAs, microRNAs and antisense RNAs, which comprise important structural and regulatory roles in the cell.

A “multi-probe library” or “library of multi-probes” comprises a plurality of multi-probes, such that the sum of the probes in the library is able to recognise a major proportion of a transcriptome, including the most abundant sequences, such that about 60%, about 70%, about 80%, about 85%, more preferably about 90%, and still more preferably 95%, of the target nucleic acids in the transcriptome, are detected by the probes.

“Sample” refers to a sample of cells, or tissue or fluid isolated from an organism or organisms, including but not limited to, for example, skin, plasma, serum, spinal fluid, lymph fluid, synovial fluid, urine, tears, blood cells, organs, tumors, and also to samples of in vitro cell culture constituents (including but not limited to conditioned medium resulting from the growth of cells in cell culture medium, recombinant cells and cell components).

The terms “Detection probes” or “detection probe” or “detection probe sequence” refer to an oligonucleotide or oligonucleotide analogue, which oligonucleotide or oligonucleotide analogue comprises a recognition sequence complementary to a nucleotide target, such as an RNA (or DNA) target sequence. It is preferable that the detection probe(s) are oligonucleotides, preferably where said recognition sequence is substituted with high-affinity nucleotide analogues, e.g. LNA, to increase the sensitivity and specificity of conventional oligonucleotides, such as DNA oligonucleotides, for hybridization to short target sequences, e.g. mature miRNAs, stem-loop precursor miRNAs, pri-miRNAs, siRNAs or other non-coding RNAs as well as miRNA binding sites in their cognate mRNA targets, mRNAs, mRNA splice variants, RNA-edited mRNAs, antisense RNAs, small nuclear RNAs (snRNA) such as small nucleolar RNAs (snoRNA).

The terms “miRNA” and “microRNA” refer to about 18-25 nt non-coding RNAs derived from endogenous genes. They are processed from longer (ca 75 nt) hairpin-like precursors termed pre-miRNAs. MicroRNAs assemble in complexes termed miRNPs and recognize their targets by antisense complementarity. If the microRNAs match 100% their target, i.e. the complementarity is complete, the target mRNA is cleaved, and the miRNA acts like a siRNA. If the match is incomplete, i.e. the complementarity is partial, then the translation of the target mRNA is blocked.

The terms “Small interfering RNAs” or “siRNAs” refer to 21-25 nt RNAs derived from processing of linear double-stranded RNA. siRNAs assemble in complexes termed RISC (RNA-induced silencing complex) and target homologous RNA sequences for endonucleolytic cleavage. Synthetic siRNAs also recruit RISCs and are capable of cleaving homologous RNA sequences

Small nucleolar RNAs (snoRNAs) are a class of small RNA molecules that guide chemical modifications (methylation or pseudouridylation) of ribosomal RNAs (rRNAs) and other RNA genes (tRNAs and other small nuclear RNAs (snRNAs)). They are classified under snRNA in MeSH. snoRNAs are commonly referred to as guide RNAs but should not be confused with the guide RNAs (gRNA) that direct RNA editing in trypanosomes.

Small nuclear RNA (snRNA) is a class of small RNA molecules that are found within the nucleus of eukaryotic cells. They are transcribed by RNA polymerase II or RNA polymerase III and are involved in a variety of important processes such as RNA splicing (removal of introns from hnRNA), regulation of transcription factors (7SK RNA) or RNA polymerase II (B2 RNA), and maintaining the telomeres. They are always associated with specific proteins, and the complexes are referred to as small nuclear ribonucleoproteins (snRNP) or sometimes as snurps. These elements are rich in uridine content.

A large group of snRNAs are known as small nucleolar RNAs (snoRNAs). These are small RNA molecules that play an essential role in RNA biogenesis and guide chemical modifications of ribosomal RNAs (rRNAs) and other RNA genes (tRNA and snRNAs). They are located in the nucleus and the cajal bodies of eukaryotic cells (the major sites of RNA synthesis).

In a preferred embodiment the snRNA is a snoRNA, such as a U6 snoRNA.

The term “piRNA” refer to small RNA molecules of up to 30 bases in length that are found in the gonads (such as the testis), and interact with the Piwi protein.

The term “RNA interference” (RNAi) refers to a phenomenon where double-stranded RNA homologous to a target mRNA leads to degradation of the targeted mRNA. More broadly defined as degradation of target mRNAs by homologous siRNAs.

The terms “microRNA precursor” or “miRNA precursor” or “pre-miRNA” refer to polynucleotide sequences (approximately 70-120 nucleotides in length) that form hairpin-like structures having a loop region and a stem region. The stem region includes a duplex created by the pairing of opposite ends of the pre-miRNA polynucleotide sequence. The loop region connects the two halves of the stem region. The pre-miRNAs are transcribed as mono- or poly-cistronic, long, primary precursor transcripts (pri-miRNAs) that are then cleaved into individual pre-miRNAs by a nuclear RNase III-like enzyme. Subsequently pre-miRNA hairpins are exported to the cytoplasm where they are processed by a second RNase III-like enzyme into miRNAs.

The “miRNA precursor loop sequence” or “loop sequence of the miRNA precursor” or “loop region” of an miRNA precursor is the portion of an miRNA precursor that is not present in the stem region and that is not retained in the mature miRNA (or its complement) upon cleavage by a RNAase III-like enzyme into miRNAs.

The “miRNA precursor stem sequence” or “stem sequence of the miRNA precursor” or “stem region” of an miRNA precursor is the portion of an miRNA precursor created by the pairing of opposite ends of the pre-miRNA polynucleotide sequence, and including the portion of the miRNA precursor that will be retained in the “mature miRNA.”

The term “Recognition sequence” refers to a nucleotide sequence that is complementary to a region within the target nucleotide sequence essential for sequence-specific hybridization between the target nucleotide sequence and the recognition sequence.

The term “label” as used herein refers to any atom or molecule which can be used to provide a detectable (preferably quantifiable) signal, and which can be attached to a nucleic acid or protein. Labels may provide signals detectable by fluorescence, radioactivity, colorimetric, X-ray diffraction or absorption, magnetism, enzymatic activity, and the like.

As used herein, the terms “nucleic acid”, “polynucleotide” and “oligonucleotide” refer to primers, probes, oligomer fragments to be detected, oligomer controls and unlabelled blocking oligomers and shall be generic to polydeoxyribonucleotides (containing 2-deoxy-D-ribose), to polyribonucleotides (containing D-ribose), and to any other type of polynucleotide which is an N glycoside of a purine or pyrimidine base, or modified purine or pyrimidine bases. There is no intended distinction in length between the term “nucleic acid”, “polynucleotide” and “oligonucleotide”, and these terms will be used interchangeably. These terms refer only to the primary structure of the molecule. Thus, these terms include double- and single-stranded DNA, as well as double- and single stranded RNA. The oligonucleotide is comprised of a sequence of approximately at least 3 nucleotides, preferably at least about 6 nucleotides, and more preferably at least about 8-30 nucleotides corresponding to a region of the designated target nucleotide sequence. “Corresponding” means identical to or complementary to the designated sequence. The oligonucleotide is not necessarily physically derived from any existing or natural sequence but may be generated in any manner, including chemical synthesis, DNA replication, reverse transcription or a combination thereof.

The terms “oligonucleotide” or “nucleic acid” intend a polynucleotide of genomic DNA or RNA, cDNA, semi synthetic, or synthetic origin which, by virtue of its origin or manipulation: (1) is not associated with all or a portion of the polynucleotide with which it is associated in nature; and/or (2) is linked to a polynucleotide other than that to which it is linked in nature; and (3) is not found in nature. Because mononucleotides are reacted to make oligonucleotides in a manner such that the 5′-phosphate of one mononucleotide pentose ring is attached to the 3′ oxygen of its neighbour in one direction via a phosphodiester linkage, an end of an oligonucleotide is referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide, also may be said to have a 5′ and 3′ ends. When two different, non-overlapping oligonucleotides anneal to different regions of the same linear complementary nucleic acid sequence, the 3′ end of one oligonucleotide points toward the 5′ end of the other; the former may be called the “upstream” oligonucleotide and the latter the “downstream” oligonucleotide.

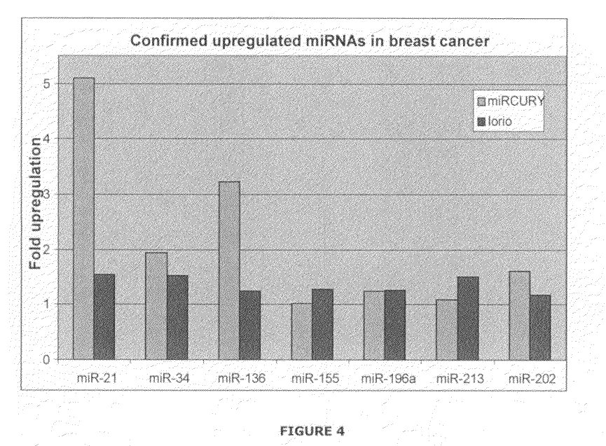

By the term “SBC nucleobases” is meant “Selective Binding Complementary” nucleobases, i.e. modified nucleobases that can make stable hydrogen bonds to their complementary nucleobases, but are unable to make stable hydrogen bonds to other SBC nucleobases. As an example, the SBC nucleobase A′, can make a stable hydrogen bonded pair with its complementary unmodified nucleobase, T. Likewise, the SBC nucleobase T′ can make a stable hydrogen bonded pair with its complementary unmodified nucleobase, A. However, the SBC nucleobases A′ and T′ will form an unstable hydrogen bonded pair as compared to the base pairs A′-T and A-T′. Likewise, a SBC nucleobase of C is designated C′ and can make a stable hydrogen bonded pair with its complementary unmodified nucleobase G, and a SBC nucleobase of G is designated G′ and can make a stable hydrogen bonded pair with its complementary unmodified nucleobase C, yet C′ and G′ will form an unstable hydrogen bonded pair as compared to the base pairs C′-G and C-G′. A stable hydrogen bonded pair is obtained when 2 or more hydrogen bonds are formed e.g. the pair between A′ and T, A and T′, C and G′, and C′ and G. An unstable hydrogen bonded pair is obtained when 1 or no hydrogen bonds is formed e.g. the pair between A′ and T′, and C′ and G′. Especially interesting SBC nucleobases are 2,6-diaminopurine (A′, also called D) together with 2-thio-uracil (U′, also called ^{2S}U)(2-thio-4-oxo-pyrimidine) and 2-thio-thymine (T′, also called ^{2S}T)(2-thio-4-oxo-5-methyl-pyrimidine). FIG. 4 in PCT Publication No. WO 2004/024314 illustrates that the pairs A-^{2S}T and D-T have 2 or more than 2 hydrogen bonds whereas the D-^{2S}T pair forms a single (unstable) hydrogen bond. Likewise the SBC nucleobases pyrrolo-[2,3-d]pyrimidine-2(3H)-one (C′, also called PyrroloPyr) and hypoxanthine (G′, also called I)(6-oxo-purine) are shown in FIG. 4 in PCT Publication No. WO 2004/024314 where the pairs PyrroloPyr-G and C—I have 2 hydrogen bonds each whereas the PyrroloPyr-I pair forms a single hydrogen bond.

“SBC LNA oligomer” refers to a “LNA oligomer” containing at least one LNA monomer where the nucleobase is a “SBC nucleobase”. By “LNA monomer with an SBC nucleobase” is meant a “SBC LNA monomer”. Generally speaking SBC LNA oligomers include oligomers that besides the SBC LNA monomer(s) contain other modified or naturally occurring nucleotides or nucleosides. By “SBC monomer” is meant a non-LNA monomer with a SBC nucleobase. By “isosequential oligonucleotide” is meant an oligonucleotide with the same sequence in a Watson-Crick sense as the corresponding modified oligonucleotide e.g. the sequences agTtcATg is equal to agTscD^{2S}Ug where s is equal to the SBC DNA monomer 2-thio-t or 2-thio-u, D is equal to the SBC LNA monomer LNA-D and ^{2S}U is equal to the SBC LNA monomer LNA ^{2S}U.

The complement of a nucleic acid sequence as used herein refers to an oligonucleotide which, when aligned with the nucleic acid sequence such that the 5′ end of one sequence is paired with the 3′ end of the other, is in “antiparallel association.” Bases not commonly found in natural nucleic acids may be included in the nucleic acids of the present invention include, for example, inosine and 7-deazaguanine. Complementarity may not be perfect; stable duplexes may contain mismatched base pairs or unmatched bases. Those skilled in the art of nucleic acid technology can determine duplex stability empirically considering a number of variables including, for example, the length of the oligonucleotide, percent concentration of cytosine and guanine bases in the oligonucleotide, ionic strength, and incidence of mismatched base pairs.

Stability of a nucleic acid duplex is measured by the melting temperature, or “Tₘ”. The Tₘ of a particular nucleic acid duplex under specified conditions is the temperature at which half of the duplexes have disassociated.

The term “nucleobase” covers the naturally occurring nucleobases adenine (A), guanine (G), cytosine (C), thymine (T) and uracil (U) as well as non-naturally occurring nucleobases such as xanthine, diaminopurine, 8-oxo-N⁶-methyladenine, 7-deazaxanthine, 7-deazaguanine, N⁴,N⁴-ethanocytosin, N⁶,N⁶-ethano-2,6-diaminopurine, 5-methylcytosine, 5-(C³-C⁶)-alkynyl-cytosine, 5-fluorouracil, 5-bromouracil, pseudoisocytosine, 2-hydroxy-5-methyl-4-triazolopyridin, isocytosine, isoguanine, inosine and the “non-naturally occurring” nucleobases described in Benner et al., U.S. Pat. No. 5,432,272 and Susan M. Freier and Karl-Heinz Altmann, Nucleic Acid Research, 25: 4429-4443, 1997. The term “nucleobase” thus includes not only the known purine and pyrimidine heterocycles, but also heterocyclic analogues and tautomers thereof. Further naturally and non naturally occurring nucleobases include those disclosed in U.S. Pat. No. 3,687,808; in chapter 15 by Sanghvi, in Antisense Research and Application, Ed. S. T. Crooke and B. Lebleu, CRC Press, 1993; in Englisch, et al., Angewandte Chemie, International Edition, 30: 613-722, 1991 (see, especially pages 622 and 623, and in the Concise Encyclopedia of Polymer Science and Engineering, J. I. Kroschwitz Ed., John Wiley & Sons, pages 858-859, 1990, Cook, Anti-Cancer DrugDesign 6: 585-607, 1991, each of which are hereby incorporated by reference in their entirety).

The term “nucleosidic base” or “nucleobase analogue” is further intended to include heterocyclic compounds that can serve as like nucleosidic bases including certain “universal bases” that are not nucleosidic bases in the most classical sense but serve as nucleosidic bases. Especially mentioned as a universal base is 3-nitropyrrole or a 5-nitroindole. Other preferred compounds include pyrene and pyridyloxazole derivatives, pyrenyl, pyrenylmethylglycerol derivatives and the like. Other preferred universal bases include, pyrrole, diazole or triazole derivatives, including those universal bases known in the art.

Preferred nucleobase analogues include, 2′-O-alkyl-RNA unit, 2′-OMe-RNA unit, 2′-amino-DNA unit, 2′-fluoro-DNA unit, LNA unit, PNA unit, HNA unit, INA unit, most preferably LNA.

By “oligonucleotide,” “oligomer,” or “oligo” is meant a successive chain of monomers (e.g., glycosides of heterocyclic bases) connected via internucleoside linkages. The linkage between two successive monomers in the oligo consist of 2 to 4, desirably 3, groups/atoms selected from —CH₂—, —O—, —S—, —NR^{H}—, >C═O, >C═NR^{H}, >C═S, —Si(R″)₂—, —SO—, —S(O)₂—, —P(O)₂—, —PO(BH₃)—, —P(O,S)—, —P(S)₂—, —PO(R″)—, —PO(OCH₃)—, and —PO(NHR^{H})—, where R^{H} is selected from hydrogen and C₁₋₄-alkyl, and R″ is selected from C₁₋₆-alkyl and phenyl. Illustrative examples of such linkages are —CH₂—CH₂—CH₂—, —CH₂—CO—CH₂—, —CH₂—CHOH—CH₂—, —O—CH₂—O—, —O—CH₂—CH₂—, —O—CH₂—CH═ (including R⁵ when used as a linkage to a succeeding monomer), —CH₂—CH₂—O—, —NR^{H}—CH₂—CH₂—, —CH₂—CH₂—NR^{H}—, —CH₂—NR^{H}—CH₂—, —O—CH₂—CH₂—NR^{H}—, —NR^{H}—CO—O—, —NR^{H}—CO—NR^{H}—, —NR^{H}—CS—NR^{H}—, —NR^{H}—C(═NR^{H})—NR^{H}—, —NR^{H}—CO—CH₂—NR^{H}—, —O—CO—O—, —O—CO—CH₂—O—, —O—CH₂—CO—O—, —CH₂—CO—NR^{H}—, —O—CO—NR^{H}—, —NR^{H}—CO—CH₂—, —O—CH₂—CO—NR^{H}—, —O—CH₂—CH₂—NR^{H}—, —CH═N—O—, —CH₂—NR^{H}—O—, —CH₂—O—N═ (including R⁵ when used as a linkage to a succeeding monomer), —CH₂—O—NR^{H}—, —CO—NR^{H}—CH₂—, —CH₂—NR^{H}—O—, —CH₂—NR^{H}—CO—, —O—NR^{H}—CH₂—, —O—NR^{H}—, —O—CH₂—S—, —S—CH₂—O—, —CH₂—CH₂—S—, —O—CH₂—CH₂—S—, —S—CH₂—CH═(including R⁵ when used as a linkage to a succeeding monomer), —S—CH₂—CH₂—, —S—CH₂—CH₂—O—, —S—CH₂—CH₂—S—, —CH₂—S—CH₂—, —CH₂—SO—CH₂—, —CH₂—SO₂—CH₂—, —O—SO—O—, —O—S(O)₂—O—, —O—S(O)₂—CH₂—, —O—S(O)₂—NR^{H}—, —NR^{H}—S(O)₂—CH₂—, —O—S(O)₂—CH₂—, —O—P(O)₂—O—, —O—P(O,S)—O—, —O—P(S)₂—O—, —S—P(O)₂—O—, —S—P(O,S)—O—, —S—P(S)₂—O—, —O—P(O)₂—S—, —O—P(O,S)—S—, —O—P(S)₂—S—, —S—P(O)₂—S—, —S—P(O,S)—S—, —S—P(S)₂—S—, —O—PO(R″)—O—, —O—PO(OCH₃)—O—, —O—PO(OCH₂CH₃)—O—, —O—PO(OCH₂CH₂S—R)—O—, —O—PO(BH₃)—O—, —O—PO(NHR^{N})—O—, —O—P(O)₂—NR^{H}—, —NR^{H}—P(O)₂—O—, —O—P(O,NR^{H})—O—, —CH₂—P(O)₂—O—, —O—P(O)₂—CH₂—, and —O—Si(R″)₂—O—; among which —CH₂—CO—NR^{H}—, —CH₂—NR^{H}—O—, —S—CH₂—O—, —O—P(O)₂—O—, —O—P(O,S)—O—, —O—P(S)₂—O—, —NR^{H}—P(O)₂—O—, —O—P(O,NR^{H})—O—, —O—PO(R″)—O—, —O—PO(CH₃)—O—, and —O—PO(NHR^{N})—O—, where R^{H} is selected form hydrogen and C₁₋₄-alkyl, and R″ is selected from C₁₋₆-alkyl and phenyl, are especially desirable. Further illustrative examples are given in Mesmaeker et. al., Current Opinion in Structural Biology 1995, 5, 343-355 and Susan M. Freier and Karl-Heinz Altmann, Nucleic Acids Research, 1997, vol 25, pp 4429-4443. The left-hand side of the internucleoside linkage is bound to the 5-membered ring as substituent P* at the 3′-position, whereas the right-hand side is bound to the 5′-position of a preceding monomer.

By “LNA” or “LNA monomer” (e.g., an LNA nucleoside or LNA nucleotide) or an LNA oligomer (e.g., an oligonucleotide or nucleic acid) is meant a nucleoside or nucleotide analogue that includes at least one LNA monomer. LNA monomers as disclosed in PCT Publication WO 99/14226 are in general particularly desirable modified nucleic acids for incorporation into an oligonucleotide of the invention. Additionally, the nucleic acids may be modified at either the 3′ and/or 5′ end by any type of modification known in the art. For example, either or both ends may be capped with a protecting group, attached to a flexible linking group, attached to a reactive group to aid in attachment to the substrate surface, etc. Desirable LNA monomers and their method of synthesis also are disclosed in U.S. Pat. No. 6,043,060, U.S. Pat. No. 6,268,490, PCT Publications WO 01/07455, WO 01/00641, WO 98/39352, WO 00/56746, WO 00/56748 and WO 00/66604 as well as in the following papers: Morita et al., Bioorg. Med. Chem. Lett. 12(1):73-76, 2002; Hakansson et al., Bioorg. Med. Chem. Lett. 11(7):935-938, 2001; Koshkin et al., J. Org. Chem. 66(25):8504-8512, 2001; Kvaerno et al., J. Org. Chem. 66(16):5498-5503, 2001; Hakansson et al., J. Org. Chem. 65(17):5161-5166, 2000; Kvaerno et al., J. Org. Chem. 65(17):5167-5176, 2000; Pfundheller et al., Nucleosides Nucleotides 18(9):2017-2030, 1999; and Kumar et al., Bioorg. Med. Chem. Lett. 8(16):2219-2222, 1998.

Preferred LNA monomers, also referred to as “oxy-LNA” are LNA monomers which include bicyclic compounds as disclosed in PCT Publication WO 03/020739 wherein the bridge between R^{4′} and R^{2′} as shown in formula (I) below together designate —CH₂—O— or —CH₂—CH₂—O—.

By “LNA modified oligonucleotide” or “LNA substituted oligonucleotide” is meant a oligonucleotide comprising at least one LNA monomer of formula (I), described infra, having the below described illustrative examples of modifications:

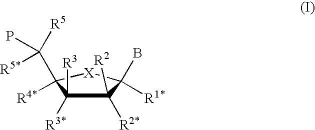

wherein X is selected from —O—, —S—, —N(R^{N})—, —C(R⁶R⁶*)—, —O—C(R⁷R⁷*)—, —C(R⁶R⁶*)—O—, —S—C(R⁷R⁷*)—, —C(R⁶R⁶*)—S—, —N(R^{N}*)—C(R⁷R⁷*)—, —C(R⁶R⁶*)—N(R^{N}*)—, and —C(R⁶R⁶*)—C(R⁷R⁷*).

B is selected from a modified base as discussed above e.g. an optionally substituted carbocyclic aryl such as optionally substituted pyrene or optionally substituted pyrenylmethylglycerol, or an optionally substituted heteroalicylic or optionally substituted heteroaromatic such as optionally substituted pyridyloxazole, optionally substituted pyrrole, optionally substituted diazole or optionally substituted triazole moieties; hydrogen, hydroxy, optionally substituted C₁₋₄-alkoxy, optionally substituted C₁₋₄-alkyl, optionally substituted C₁₋₄-acyloxy, nucleobases, DNA intercalators, photochemically active groups, thermochemically active groups, chelating groups, reporter groups, and ligands.

P designates the radical position for an internucleoside linkage to a succeeding monomer, or a 5′-terminal group, such internucleoside linkage or 5′-terminal group optionally including the substituent R⁵. One of the substituents R², R²*, R³, and R³* is a group P* which designates an internucleoside linkage to a preceding monomer, or a 2′/3′-terminal group. The substituents of R¹*, R⁴*, R⁵, R⁵*, R⁶, R⁶*, R⁷, R⁷*, R^{N}, and the ones of R², R²*, R³, and R³* not designation P* each designates a biradical comprising about 1-8 groups/atoms selected from —C(R^{a}R^{b})—, —C(R^{a})═C(R^{a})—, —C(R^{a})═N—, —C(R^{a})—O—, —O—, —Si(R^{a})₂—, —C(R^{a})—S, —S—, —SO₂—, —C(R^{a})—N(R^{b})—, —N(R^{a})—, and >C═Q, wherein Q is selected from —O—, —S—, and —N(R^{a})—, and R^{a} and R^{b} each is independently selected from hydrogen, optionally substituted C₁₋₁₂-alkyl, optionally substituted C₂₋₁₂-alkenyl, optionally substituted C₂₋₁₂-alkynyl, hydroxy, C₁₋₁₂-alkoxy, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, hetero-aryloxy-carbonyl, heteroaryloxy, heteroarylcarbonyl, amino, mono- and di(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C-₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl may be optionally substituted, and where two geminal substituents R^{a} and R^{b} together may designate optionally substituted methylene (═CH₂), and wherein two non-geminal or geminal substituents selected from R^{a}, R^{b}, and any of the substituents R¹*, R², R²*, R³, R³*, R⁴*, R⁵, R⁵*, R⁶ and R⁶*, R⁷, and R⁷* which are present and not involved in P, P* or the biradical(s) together may form an associated biradical selected from biradicals of the same kind as defined before; the pair(s) of non-geminal substituents thereby forming a mono- or bicyclic entity together with (i) the atoms to which said non-geminal substituents are bound and (ii) any intervening atoms.

Each of the substituents R¹*, R², R²*, R³, R⁴*, R⁵, R⁵*, R⁶ and R⁶*, R⁷, and R⁷* which are present and not involved in P, P* or the biradical(s), is independently selected from hydrogen, optionally substituted C₁₋₁₂-alkyl, optionally substituted C₂₋₁₂-alkenyl, optionally substituted C₂₋₁₂-alkynyl, hydroxy, C₁₋₁₂-alkoxy, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di-(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl may be optionally substituted, and where two geminal substituents together may designate oxo, thioxo, imino, or optionally substituted methylene, or together may form a spiro biradical consisting of a 1-5 carbon atom(s) alkylene chain which is optionally interrupted and/or terminated by one or more heteroatoms/groups selected from —O—, —S—, and —(NR^{N})— where R^{N} is selected from hydrogen and C₁₋₄-alkyl, and where two adjacent (non-geminal) substituents may designate an additional bond resulting in a double bond; and R^{N}*, when present and not involved in a biradical, is selected from hydrogen and C₁₋₄-alkyl; and basic salts and acid addition salts thereof.

Exemplary 5′, 3′, and/or 2′ terminal groups include —H, —OH, halo (e.g., chloro, fluoro, iodo, or bromo), optionally substituted aryl, (e.g., phenyl or benzyl), alkyl (e.g., methyl or ethyl), alkoxy (e.g., methoxy), acyl (e.g. acetyl or benzoyl), aroyl, aralkyl, hydroxy, hydroxyalkyl, alkoxy, aryloxy, aralkoxy, nitro, cyano, carboxy, alkoxycarbonyl, aryloxycarbonyl, aralkoxycarbonyl, acylamino, aroylamino, alkylsulfonyl, arylsulfonyl, heteroarylsulfonyl, alkylsulfinyl, arylsulfinyl, heteroarylsulfinyl, alkylthio, arylthio, heteroarylthio, aralkylthio, heteroaralkylthio, amidino, amino, carbamoyl, sulfamoyl, alkene, alkyne, protecting groups (e.g., silyl, 4,4′-dimethoxytrityl, monomethoxytrityl, or trityl(triphenylmethyl)), linkers (e.g., a linker containing an amine, ethylene glycol, quinone such as anthraquinone), detectable labels (e.g., radiolabels or fluorescent labels), and biotin.

It is understood that references herein to a nucleic acid unit, nucleic acid residue, LNA monomer, or similar term are inclusive of both individual nucleoside units and nucleotide units and nucleoside units and nucleotide units within an oligonucleotide.

A “modified base” or other similar terms refer to a composition (e.g., a non-naturally occurring nucleobase or nucleosidic base), which can pair with a natural base (e.g., adenine, guanine, cytosine, uracil, and/or thymine) and/or can pair with a non-naturally occurring nucleobase or nucleosidic base. Desirably, the modified base provides a Tₘ differential of 15, 12, 10, 8, 6, 4, or 2° C. or less as described herein. Exemplary modified bases are described in EP 1 072 679 and WO 97/12896.

The term “chemical moiety” refers to a part of a molecule. “Modified by a chemical moiety” thus refer to a modification of the standard molecular structure by inclusion of an unusual chemical structure. The attachment of said structure can be covalent or non-covalent.

The term “inclusion of a chemical moiety” in an oligonucleotide probe thus refers to attachment of a molecular structure. Such as chemical moiety include but are not limited to covalently and/or non-covalently bound minor groove binders (MGB) and/or intercalating nucleic acids (INA) selected from a group consisting of asymmetric cyanine dyes, DAPI, SYBR Green I, SYBR Green II, SYBR Gold, PicoGreen, thiazole orange, Hoechst 33342, Ethidium Bromide, 1-O-(1-pyrenylmethyl)glycerol and Hoechst 33258. Other chemical moieties include the modified nucleobases, nucleosidic bases or LNA modified oligonucleotides.

“Oligonucleotide analogue” refers to a nucleic acid binding molecule capable of recognizing a particular target nucleotide sequence. A particular oligonucleotide analogue is peptide nucleic acid (PNA) in which the sugar phosphate backbone of an oligonucleotide is replaced by a protein like backbone. In PNA, nucleobases are attached to the uncharged polyamide backbone yielding a chimeric pseudopeptide-nucleic acid structure, which is homomorphous to nucleic acid forms.

“High affinity nucleotide analogue” or “affinity-enhancing nucleotide analogue” refers to a non-naturally occurring nucleotide analogue that increases the “binding affinity” of an oligonucleotide probe to its complementary recognition sequence when substituted with at least one such high-affinity nucleotide analogue.

As used herein, a probe with an increased “binding affinity” for a recognition sequence compared to a probe which comprises the same sequence but does not comprise a stabilizing nucleotide, refers to a probe for which the association constant (Kₐ) of the probe recognition segment is higher than the association constant of the complementary strands of a double-stranded molecule. In another preferred embodiment, the association constant of the probe recognition segment is higher than the dissociation constant (K_{d}) of the complementary strand of the recognition sequence in the target sequence in a double stranded molecule.

Monomers are referred to as being “complementary” if they contain nucleobases that can form hydrogen bonds according to Watson-Crick base-pairing rules (e.g. G with C, A with T or A with U) or other hydrogen bonding motifs such as for example diaminopurine with T, 5-methyl C with G, 2-thiothymidine with A, inosine with C, pseudoisocytosine with G, etc.

Oligonucleotides are referred to as being “complementary” if they contain a contiguous stretch of monomers which are complementary to the target sequence—the contiguous stretch is typically at least 8, such as at least 9, such as at least 10, such as at least 11, such as at least 12, such as at least 13, such as at least 14, such as at least 15, such as at least 16, such as at least 17, such as at least 18 nucleobases which are complementary to the target sequence. Typically a complementary contiguous stretch may comprise no more than a single mismatch with the target sequence.

The term “preceding monomer” relates to the neighbouring monomer in the 5′-terminal direction and the “succeeding monomer” relates to the neighbouring monomer in the 3′-terminal direction.

The term “target nucleic acid” or “target ribonucleic acid” refers to any relevant nucleic acid of a single specific sequence, e. g., a biological nucleic acid, e. g., derived from a patient, an animal (a human or non-human animal), a cell, a tissue, an organism, etc. In one embodiment, the target nucleic acid is derived from a patient, e.g., a human patient. In this embodiment, the invention optionally further includes selecting a treatment, diagnosing a disease, or diagnosing a genetic predisposition to a disease, based upon detection of the target nucleic acid.

“Target sequence” refers to a specific nucleic acid sequence within any target nucleic acid.

The term “stringent conditions”, as used herein, is the “stringency” which occurs within a range from about Tₘ-5° C. (5° C. below the melting temperature (Tₘ) of the probe) to about 20° C. to 25° C. below Tₘ. As will be understood by those skilled in the art, the stringency of hybridization may be altered in order to identify or detect identical or related polynucleotide sequences. Hybridization techniques are generally described in Nucleic Acid Hybridization, A Practical Approach, Ed. Hames, B. D. and Higgins, S. J., IRL Press, 1985; Gall and Pardue, Proc. Natl. Acad. Sci., USA 63: 378-383, 1969; and John, et al. Nature 223: 582-587, 1969.

### DETAILED DESCRIPTION OF THE INVENTION

Method for the Characterisation of Cancer

The invention provides a method for the characterisation of cancer. The data obtained by the method can be used to provide information on one or more features of cancer.

The terms cancer and tumor can be used interchangeably herein. This is to say that although not all tumors are cancerous, the methods of the invention may be used to characterize tumors which are cancerous (malignant) or non-cancerous (benign). It is also recognized that not all cancers are tumors—however, it in a preferred aspect the cancer is a tumor.

The at least one feature of the cancer which is characterized by the method according to the invention may be selected from one or more of the following:

Diagnosis of cancer, the signal data can be used to determine whether the test sample comprises cells that are cancerous (i.e. presence or absence of cancer).

The prognosis of the cancer, such as the speed at which the cancer may develop and or metastasize (i.e. spread from one part of the body to another or the expected life expectancy of the patient with said cancer (such as less than five years, or greater than five years). In one embodiment the prognosis may be that the life expectancy of the patient is less than 5 years, such as less than 4 years, less than 3 years, less than two years, less than 1 year, less than six months or less than 3 months.

The origin of said cancer, this may be the cause of the cancer, or in the case of secondary cancer, the origin of the primary cancer. The origin may for example be selected from the following lists of cancer types.

The type of said cancer, such as a cancer selected from the group consisting of the following: A solid tumor; ovarian cancer, breast cancer, non-small cell lung cancer, renal cell cancer, bladder cancer, esophagus cancer, stomach cancer, prostate cancer, pancreatic cancer, lung cancer, cervical cancer, colon cancer, colorectal cancer. In a preferred embodiment the cancer is breast cancer.

The type of cancer may be selected from the group consisting of: A carcinoma, such as a carcinoma selected from the group consisting of ovarian carcinoma, breast carcinoma, non-small cell lung cancer, renal cell carcinoma, bladder carcinoma, recurrent superficial bladder cancer, stomach carcinoma, prostatic carcinoma, pancreatic carcinoma, lung carcinoma, cervical carcinoma, cervical dysplasia, laryngeal papillomatosis, colon carcinoma, colorectal carcinoma, carcinoid tumors. A basal cell carcinoma; A malignant melanoma, such as a malignant melanoma selected from the group consisting of superficial spreading melanoma, nodular melanoma, lentigo maligna melanoma, acral melagnoma, amelanotic melanoma and desmoplastic melanoma; A sarcoma, such as a sarcoma selected from the group consisting of osteosarcoma, Ewing's sarcoma, chondrosarcoma, malignant fibrous histiocytoma, fibrosarcoma and Kaposi's sarcoma; and a glioma. In a preferred embodiment, the cancer is a breast carcinoma.

The use of non-coding RNA markers for determining the origin of cells is disclosed in U.S. application Ser. No. 11/324,177, which is hereby incorporated by reference.

Cancer of unknown primary site is a common clinical entity, accounting for 2% of all cancer diagnoses in the Surveillance, Epidemiology, and End Results (SEER) registries between 1973 and 1987 (C. Muir. Cancer of unknown primary site Cancer 1995. 75: 353-356). In spite of the frequency of this syndrome, relatively little attention has been given to this group of patients, and systematic study of the entity has lagged behind that of other areas in oncology. Widespread pessimism concerning the therapy and prognosis of these patients has been the major reason for the lack of effort in this area. The patient with carcinoma of unknown primary site is commonly stereotyped as an elderly, debilitated individual with metastases at multiple visceral sites. Early attempts at systemic therapy yielded low response rates and had a negligible effect on survival, thereby strengthening arguments for a nihilistic approach to these patients. The heterogeneity of this group has also made the design of therapeutic studies difficult; it is well recognized that cancers with different biologies from many primary sites are represented. In the past 10 years, substantial improvements have been made in the management and treatment of some patients with carcinoma of unknown primary site. The identification of treatable patients within this heterogeneous group has been made possible by the recognition of several clinical syndromes that predict chemotherapy responsiveness, and also by the development of specialized pathologic techniques that can aid in tumor characterization. Therefore, the optimal management of patients with cancer of unknown primary site now requires appropriate clinical and pathologic evaluation to identify treatable subgroups, followed by the administration of specific therapy. Many patients with adenocarcinoma of unknown primary site have widespread metastases and poor performance status at the time of diagnosis. The outlook for most of these patients is poor, with median survival of 4 to 6 months. However, subsets of patients with a much more favorable outlook are contained within this large group, and optimal initial evaluation enables the identification of these treatable subsets. In addition, empiric chemotherapy incorporating newer agents has produced higher response rates and probably improves the survival of patients with good performance status.

Fine-needle aspiration biopsy (FNA) provides adequate amounts of tissue for definitive diagnosis of poorly differentiated tumors, and identification of the primary source in about one fourth of cases (C. V. Reyes, K. S. Thompson, J. D. Jensen, and A. M. Chouelhury. Metastasis of unknown origin: the role of fine needle aspiration cytology Diagn Cytopathol 1998. 18: 319-322).

microRNAs have emerged as important non-coding RNAs, involved in a wide variety of regulatory functions during cell growth, development and differentiation. Some reports clearly indicate that microRNA expression may be indicative of cell differentiation state, which again is an indication of organ or tissue specification. Therefore a catalogue of miRNA tissue expression profiles may serve as the basis for a diagnostic tool determining the tissue origin of tumors of unknown origin. So, since it is possible to map non-coding RNAs, such as miRNAs and snRNAs in cells vs. the tissue origin of cell, the present invention presents a convenient means for detection of tissue origin of such tumors.

The present inventors have discovered that small-nucleolar RNAs also constitute an important class of non-coding RNAs.

Hence, the present invention in general relates to a method for determining tissue origin of tumors comprising probing cells of the tumor with a collection of probes which is capable of mapping non-coding RNAs, such as miRNAs and snRNAs to a tissue origin.

non-coding RNA (such as miRNAs and snRNAs) typing according to the principles of the present example can be applied to RNA from a variety of normal tissues and tumor tissues (of known origin) and over time a database is build up, which consists of non-coding RNAs (such as miRNAs and snRNAs) expression profiles from normal and tumor tissue. When subjecting RNA from a tumor tissue sample, the resulting non-coding RNA (such as miRNAs and snRNAs) profile can be analysed for its degree of identity with each of the profiles of the database—the closest matching profiles are those having the highest likelihood of representing a tumor having the same origin (but also other characteristics of clinical significance, such as degree of malignancy, prognosis, optimum treatment regimen and prediction of treatment success). The non-coding RNA (such as miRNAs and snRNAs) profile may of course be combined with other tumor origin determination techniques, cf. e.g. Xiao-Jun Ma et al., Arch Pathol Lab Med 130, 465-473, which demonstrates molecular classification of human cancers into 39 tumor classes using a microarray designed to detect RT-PCR amplified mRNA derived from expression of 92 tumor-related genes. The presently presented technology allows for an approach which is equivalently safe for the use of a non-coding RNA (such as miRNAs and snRNAs) detection assay instead of a mRNA detection assay.

The invention provides a method of characterising a tumor of unknown origin, such as a metastasis, or putative metastasis, wherein at least one non-coding RNA (such as miRNAs and snRNAs) species is detected in a sample of RNA from a tumor, (i.e. a first population of target molecules obtained from at least one test sample) thus providing a non-coding RNA (such as miRNAs and snRNAs) expression profile from the tumor, and subsequently comparing said miRNA expression profile with previously established non-coding RNA (such as miRNAs and snRNAs) expression profiles from normal tissue and/or tumor tissue.

In one embodiment, the tumor may be a breast tumor, or it may be derived from a breast tumor.

The RNA may be total RNA isolated from the tumor, or a purified fraction thereof.

In one embodiment, the non-coding RNA (such as snRNA and miRNA) expression profile from the tumor and the previously established miRNA expression profiles provides for an indication of the origin of the tumor, the patient's prognosis, the optimum treatment regimen of the tumor and/or a prediction of the outcome of a given anti-tumor treatment.

The therapy outcome prediction, such as a prediction of the responsiveness of the cancer to chemotherapy and/or radiotherapy and/or the suitability of said cancer to hormone treatment, and such as the suitability of said cancer for removal by invasive surgery. In one embodiment, the therapy outcome predication may be the prediction of the suitability of the treatment of the cancer to combined adjuvant therapy.

The therapy may be herceptin, which is frequently used for the treatment of estrogen receptor positive cancers (such as breast cancer).

The Patient and Test Sample

Suitable samples may comprise a wide range of mammalian and human cells, including protoplasts; or other biological materials, which may harbour target nucleic acids. The methods are thus applicable to tissue culture mammalian cells, mammalian cells (e.g., blood, serum, plasma, reticulocytes, lymphocytes, urine, bone marrow tissue, cerebrospinal fluid or any product prepared from blood or lymph) or any type of tissue biopsy (e.g. a muscle biopsy, a liver biopsy, a kidney biopsy, a bladder biopsy, a bone biopsy, a cartilage biopsy, a skin biopsy, a pancreas biopsy, a biopsy of the intestinal tract, a thymus biopsy, a mammae biopsy, a uterus biopsy, a testicular biopsy, an eye biopsy or a brain biopsy, e.g., homogenized in lysis buffer), and archival tissue nucleic acids.

The test sample is typically obtained from a patient that has or is suspected of having cancer, such as breast cancer, or who is suspected of having a high risk of developing cancer. The method can, therefore be undertaken as a precautionary matter in the prevention of, or early diagnosis of cancer.

The patient (or organism) is a mammal, preferably a human being. The patient may be male or female, although this may depend on the type of tissue/cancer being investigated (e.g. ovarian cancer effects only women).

The test sample is typically obtained from the patient by biopsy or tissue sampling. When referring to the signal obtained from a test (or control) sample, it refers to the signal obtained from the hybridization using the first (or further) population of molecules prepared from the test (or control) sample.

The Control Sample

In one embodiment, the control sample may be obtained from the same patient at the same time that the test sample is taken. In one embodiment, the control sample may be a sample taken previously, e.g. a sample of the same or a different cancer/tumor, the comparison of which may, for example, provide characterisation of the source of the new tumor, or progression of the development of an existing cancer, such as before, during or after treatment.

In one embodiment, the control sample may be taken from healthy tissue, for example tissue taken adjacent to the cancer, such as within 1 or 2 cm diameter from the external edge of said cancer. Alternatively the control sample may be taken from an equivalent position in the patients body, for example in the case of breast cancer, tissue may be taken from the breast which is not cancerous.

In one embodiment, the control sample may also be obtained from a different patient, e.g. it may be a control sample, or a collection of control samples, representing different types of cancer, for example those listed herein (i.e. cancer reference samples). Comparison of the test sample data with data obtained from such cancer reference samples may for example allow for the characterization of the test cancer to a specific type and/or stage of cancer.

In one embodiment, at least one control sample is obtained, and a second population of nucleic acids from the at least one control sample is, in addition to the test sample, presented and hybridized against at least one detection probe.

The detection probe target for the test and control sample may be the same, the ratio of the signal obtained between the control and test sample being indicative of a differential quantification of the target.

In one embodiment, the control sample may be obtained from the same patient as the test sample.

In one embodiment, the control sample may be obtained from a non tumorous tissue, such as from tissue adjacent to said putative tumor, and/or from an equivalent position elsewhere in the body.

In one embodiment, the control sample may be obtained from a tumor tissue. In this embodiment, there may be one or more control samples, e.g. a panel of control samples which represent one or more tumor types. Thereby allowing comparison of the test sample, with on or more control samples which have a defined origin. Such control samples, such as a panel of control samples is particularly useful when determining the origin of a cancer (e.g. metestasis) of unknown origin. Such control samples may be selected from one or more of the following: A solid tumor; ovarian cancer, breast cancer, non-small cell lung cancer, renal cell cancer, bladder cancer, esophagus cancer, stomach cancer, prostate cancer, pancreatic cancer, lung cancer, cervical cancer, colon cancer, colorectal cancer; Such control samples may also be selected from one or more of the following: The type of cancer may be selected from the group consisting of: A carcinoma, such as a carcinoma selected from the group consisting of ovarian carcinoma, breast carcinoma, non-small cell lung cancer, renal cell carcinoma, bladder carcinoma, recurrent superficial bladder cancer, stomach carcinoma, prostatic carcinoma, pancreatic carcinoma, lung carcinoma, cervical carcinoma, cervical dysplasia, laryngeal papillomatosis, colon carcinoma, colorectal carcinoma, carcinoid tumors. A basal cell carcinoma; A malignant melanoma, such as a malignant melanoma selected from the group consisting of superficial spreading melanoma, nodular melanoma, lentigo maligna melanoma, acral melagnoma, amelanotic melanoma and desmoplastic melanoma; A sarcoma, such as a sarcoma selected from the group consisting of osteosarcoma, Ewing's sarcoma, chondrosarcoma, malignant fibrous histiocytoma, fibrosarcoma and Kaposi's sarcoma; and a glioma.

In one embodiment, the hybridization signal obtained from the test sample is higher than the hybridization signal obtained from the control sample.

In one embodiment, the hybridization signal obtained from the test sample is lower than the hybridization signal obtained from the control sample.

In one embodiment, at least two control samples are obtained, one control sample being obtained from said patient (see above), and at least one further control sample being obtained from a previously obtained sample of a cancer, such as a cancer of the same type as the test sample, or a different cancer such as those herein listed. The cancer may originate from the same patient or a different patient.

In one embodiment, the hybridization signal obtained from the at least one further test sample is equivalent to or greater than the signal obtained from either the signal obtained from the first control sample and/or the signal obtained from the test sample.

In one embodiment, the hybridization signal obtained from the at least one further test sample is less than the signal obtained from either the signal obtained from the first control sample and/or the signal obtained from the test sample.

In one embodiment, the test and control samples are hybridized to said at least one detection probe simultaneously, either in parallel hybridizations or in the same hybridization experiment.

In one embodiment, the test and control sample or samples are hybridized to said at least one detection probe sequentially, either in the same hybridization experiment, or different hybridization experiments.

The RNA Fraction

In one embodiment, the RNA fraction may remain within the test sample, such as remain in the cells of the the biopsy or tissue sample, for example for in situ hybridization. The cells may still be living, or they may be dead. The cells may also be prepared for in situ hybridization using methods known in the art, e.g. they may be treated with an agent to improve permeability of the cells; the cells may also be fixed or partially fixed.

The RNA fraction may be isolated from the test sample, such as a tissue sample.

The RNA fraction preferably comprises small RNAs such as those less than 100 bases in length. The RNA fraction preferably comprises snRNAs, miRNAs and/or siRNAs and/or piRNAs.

In one embodiment, the RNA fraction comprised snRNAs.

The RNA fraction may also comprise other nucleic acids, for example the RNA fraction may be part of a total nucleic acid fraction which also comprises DNA, such as genomic and/or mitochondrial DNA. The RNA fraction may be purified. Care should be taken during RNA extraction to ensure at least a proportion of the non-ncoding RNAs, such as snRNA, miRNA and siRNAs are retained during the extraction. Suitably, specific protocols for obtaining RNA fractions comprising or enriched with small RNAs, such as snRNA or miRNAs may be used. The RNA fraction may undergo further purification to obtain an enriched RNA fraction, for example an RNA fraction enriched for non-coding RNAs. This can be achieved, for example, by removing mRNAs by use of affinity purification, e.g. using an oligodT column. RNA fractions enriched in snRNA, miRNA and siRNA may be obtained using. In one embodiment the RNA fraction is not isolated from the test sample, for example when in situ hybridization is performed, the RNA fraction remains in situ in the test sample, and the detection probes, typically labelled detection probes, are hybridized to a suitably prepared test sample.

In one embodiment the RNA fraction is used directly in the hybridization with the at least one detection probe.

The RNA fraction may comprise the target molecule, e.g. the RNA fraction obtained from a test sample, the presence of the target molecule within the RNA fraction may indicate a particular feature of a cancer. Alternatively the RNA fraction may not comprise the target molecule, e.g. the RNA fraction obtained from a test sample, the absence of the target (complementary) molecule within the RNA fraction may indicate a particular feature of a cancer.

The RNA fraction comprises non-coding RNA such as noncoding RNA selection from the group consisting of microRNA (miRNA), siRNA, piRNA and snRNA.

In one embodiment, prior to (or even during) said hybridization, the RNA fraction may be used as a template to prepare a complement of the RNA present in the fraction, said compliment may be synthesised by template directed assembly of nucleoside, nucleotide and/or nucleotide analogue monomers, to produce, for example an oligonucleotide, such as a DNA oligonucleotide. The complement may be further copied and replicated. The complement may represent the entire template RNA molecule, or may represent a population of fragments of template molecules, such as fragments that, preferably in average, retain at least 8 consecutive nucleoside units of said RNA template, such as at least 12 of said units or at least 14 of said units. It is preferred that at least 8 consecutive nucleoside units of said complementary target, such as at least 12 of said units or at least 14 of said units of said complementary target are retained. When the complementary target is a precursor RNA, or a molecule derived therefore, it is preferred that at least part of the loop structure of the precursor molecule is retained, as this will allow independent detection over the mature form of the non-coding RNA, or molecule derived therefrom.

Therefore, in one embodiment the RNA fraction itself is not used in the hybridization, but a population of molecules, such as a population of oligonucleotides which are derived from said RNA fraction, and retain sequence information contained within said RNA fraction, are used. It is envisaged that the population of molecules derived from said RNA fraction may be further manipulated or purified prior to the hybridization step—for example they may be labelled, or a sub-fraction may be purified therefrom.

The target molecule (complementary target) may therefore be derived from RNA, but may actually comprise an alternative oligo backbone, for example DNA. The target molecule may, therefore also be a complement to the original RNA molecule, or part of the original RNA molecule from which it is derived.

In one embodiment, the RNA fraction is analyzed and the population of target RNAs and optionally control nucleic acids are determined. For example the RNA fraction, or a nucleic acid fraction derived therefrom may be undergo quantitative analysis for specific target and control sequences, for example using oligonucleotide based sequencing, such as oligonucleotide microarray hybridization. The data from the quantative analysis may then be used in a virtual hybridization with a detection probe sequence.

Hybridization

Hybridization refers to the bonding of two complementary single stranded nucleic acid polymers (such as oligonucleotides), such as RNA, DNA or polymers comprising or consisting of nucleotide analogues (such as LNA oligonucleotides). Hybridization is highly specific, and may be controlled by regulation of the concentration of salts and temperature. Hybridization occurs between complementary sequences, but may also occur between sequences which comprise some mismatches. The probes used in the methods of the present invention may, therefore be 100% complementary to the target molecule. Alternatively, in one embodiment the detection probes may comprise one or two mismatches. Typically a single mismatch will not unduly affect the specificity of binding, however two or more mismatches per 8 nucleotide residues usually prevents specific binding of the detection probe to the target species. The position of the mismatch may also be of importance, and as such the use of mismatches may be used to determine the specificity and strength of binding to target RNAs, or to allow binding to more than one allelic variant of mutation of a target species.

In one embodiment, the detection probe consists of no more than 1 mismatch.

In one embodiment, the detection probe consists of no more than 1 mismatch per 8 nucleotide/nucleotiude analogue bases.

In one embodiment, hybridization may also occur between a single stranded target molecule, such as a miRNA, siRNA piRNA, or snRNA, and a probe which comprises a complementary surface to the said target molecule, in this respect, it is the ability of the probe to form the specific bonding pattern with the target which is important.

Suitable methods for hybridization include RNA in-situ hybridization, dot blot hybridization, reverse dot blot hybridization, northern blot analysis, RNA protection assays, or expression profiling by microarrays. Such methods are standard in the art.

In one embodiment, the detection probe is capable of binding to the target non-coding RNA sequence under stringent conditions, or under high stringency conditions.

Exiqon (Denmark) provide microarrays suitable for use in the methods of the invention (microRNA Expression Profiling with miRCURY™ LNA Array).

The detection probe, such as each member of a collection of detection probes, may be bound (such as conjugated) to a bead. Luminex (Texas, USA) provides multiplex technology to allow the use of multiple detection probes to be used in a single hybridization experiment. See also Panomics QuantigenePlex™ (http://www.panomics.com/pdf/qgplexbrochure.pdf).

Suitable techniques for performing in situ hybridization are disclosed in PCT/DK2005/000838

PCR Hybridization

Whilst it is recognised that many of the short noncoding RNAs which are targets for the detection probes are too short to be detected by amplification by standard PCR, methods of amplifying such short RNAs are disclosed in WO2005/098029. Therefore, the hybridization may occur during PCR, such as RT-PCT or quantative PCR (q-PCR).

However, in one embodiment, the hybridization step does not comprise PCR such as RT-PCR or q-pCR.

Detection Probe and Recognition Sequence

Each detection probe comprises a recognition sequence consisting of nucleobases or equivalent molecule entities.

In one embodiment, the detection probes are capable of hybridizing, such as under stringent conditions or high stringency conditions to a target sequence selected from the group consisting of: SEQ ID No. 4; SEQ ID No. 72; SEQ ID No. 36; SEQ ID No. 29; SEQ ID No. 44; SEQ ID No. 65; SEQ ID No. 76; SEQ ID No. 12; SEQ ID No. 28; SEQ ID No. 83; SEQ ID No. 52; SEQ ID No. 75; SEQ ID No. 91; SEQ ID No. 9; SEQ ID No. 85; SEQ ID No. 92; SEQ ID No. 26; SEQ ID No. 14; SEQ ID No. 46; SEQ ID No. 39; SEQ ID No. 69; SEQ ID No. 66; SEQ ID No. 6; SEQ ID No. 64; SEQ ID No. 84; SEQ ID No. 93; SEQ ID No. 54; SEQ ID No. 24; SEQ ID No. 42; SEQ ID No. 94; SEQ ID No. 95; SEQ ID No. 18; SEQ ID No. 90; SEQ ID No. 87; SEQ ID No. 6; SEQ ID No. 82; SEQ ID No. 23; SEQ ID No. 55; SEQ ID No. 57; SEQ ID No. 33; SEQ ID No. 88; SEQ ID No. 37; SEQ ID No. 96; SEQ ID No. 97; SEQ ID No. 85; SEQ ID No. 55; SEQ ID No. 53; SEQ ID No. 58; SEQ ID No. 68; SEQ ID No. 59; SEQ ID No. 73; SEQ ID No. 41; SEQ ID No. 19; SEQ ID No. 67; SEQ ID No. 89; SEQ ID No. 76; SEQ ID No. 45; SEQ ID No. 63; SEQ ID No. 25; SEQ ID No. 62; SEQ ID No. 21; SEQ ID No. 78; SEQ ID No. 13; SEQ ID No. 50; SEQ ID No. 3; SEQ ID No. 27; SEQ ID No. 10; SEQ ID No. 38; SEQ ID No. 47; SEQ ID No. 77; SEQ ID No. 51; SEQ ID No. 11; SEQ ID No. 30; SEQ ID No. 43; SEQ ID No. 22; SEQ ID No. 1; SEQ ID No. 40; SEQ ID No. 48; SEQ ID No 111; SEQ ID No 112; SEQ ID No 113; and SEQ ID No. 32; SEQ ID No 219; SEQ ID No 220; SEQ ID No 221; SEQ ID No 222; SEQ ID No 223; SEQ ID No 224; SEQ ID No 225; SEQ ID No 226; SEQ ID No 227; SEQ ID No 349; SEQ ID No 350; SEQ ID No 351; SEQ ID No 352; SEQ ID No 353; SEQ ID No 354; SEQ ID No 355; SEQ ID No 356; SEQ ID No 357; SEQ ID No 358; SEQ ID No 359; SEQ ID No 360; SEQ ID No 361; SEQ ID No 362; SEQ ID No 363; SEQ ID No 364; SEQ ID No 365; SEQ ID No 366; and SEQ ID No 367; and allelic variants thereof. These sequences are referably precursor sequences which are further processed to form mature non-coding RNAs.

In a preferred embodiment, the detection probes are capable of hybridizing, such as under stringent conditions or high stringency conditions to a target sequence selected from the group consisting of: SEQ ID No. 4; SEQ ID No. 72; SEQ ID No. 36; SEQ ID No. 29; SEQ ID No. 44; SEQ ID No. 65; SEQ ID No. 76; SEQ ID No. 12; SEQ ID No. 28; SEQ ID No. 83; SEQ ID No. 52; SEQ ID No. 75; SEQ ID No. 91; SEQ ID No. 9; SEQ ID No. 85; SEQ ID No. 92; SEQ ID No. 26; SEQ ID No. 14; SEQ ID No. 46; SEQ ID No. 39; SEQ ID No. 69; SEQ ID No. 66; SEQ ID No. 6; SEQ ID No. 64; SEQ ID No. 84; SEQ ID No. 93; SEQ ID No. 54; SEQ ID No. 24; SEQ ID No. 42; SEQ ID No. 94; SEQ ID No. 95; SEQ ID No. 18; SEQ ID No. 90; SEQ ID No. 87; SEQ ID No. 6; SEQ ID No. 82; SEQ ID No. 23; SEQ ID No. 55; SEQ ID No. 57; SEQ ID No. 33; SEQ ID No. 88; SEQ ID No. 37; SEQ ID No. 96; SEQ ID No. 97; SEQ ID No. 85; SEQ ID No. 55; SEQ ID No. 53; SEQ ID No. 58; SEQ ID No. 68; SEQ ID No. 59; SEQ ID No. 73; SEQ ID No. 41; SEQ ID No. 19; SEQ ID No. 67; SEQ ID No. 89; SEQ ID No. 76; SEQ ID No. 45; SEQ ID No. 63; SEQ ID No. 25; SEQ ID No. 62; SEQ ID No. 21; SEQ ID No. 78; SEQ ID No. 13; SEQ ID No. 50; SEQ ID No. 3; SEQ ID No. 27; SEQ ID No. 10; SEQ ID No. 38; SEQ ID No. 47 (V. PREF); SEQ ID No. 77; SEQ ID No. 51; SEQ ID No. 11; SEQ ID No. 30; SEQ ID No. 43; SEQ ID No. 22; SEQ ID No. 1; SEQ ID No. 40; SEQ ID No. 48; SEQ ID No 111; SEQ ID No 112; SEQ ID No 113; and SEQ ID No. 32; SEQ ID No 219; SEQ ID No 220; SEQ ID No 221; SEQ ID No 222; SEQ ID No 223; SEQ ID No 224; SEQ ID No 225; SEQ ID No 226; SEQ ID No 227; and allelic variants thereof, such as more preferably, SEQ ID 45; SEQ ID 13; SEQ ID 113; SEQ ID No 219; SEQ ID No 220; SEQ ID No 221; SEQ ID No 222; SEQ ID No 223; SEQ ID No 224; SEQ ID No 225; SEQ ID No 226; SEQ ID No 227; and natural allelic variants thereof.

Alternatively, or in addition (for example in the case of detection probe pairs), one or more of non-coding RNAs are selected from the group consisting of: SEQ ID No 237; SEQ ID No 238; SEQ ID No 239; SEQ ID No 240; SEQ ID No 241; SEQ ID No 242; SEQ ID No 243; SEQ ID No 244; SEQ ID No 245; SEQ ID No 246; SEQ ID No 247; SEQ ID No 248; SEQ ID No 249; SEQ ID No 250; SEQ ID No 251; SEQ ID No 252; SEQ ID No 253; SEQ ID No 254; SEQ ID No 255; SEQ ID No 256; SEQ ID No 257; SEQ ID No 258; SEQ ID No 259; SEQ ID No 260; SEQ ID No 261; SEQ ID No 262; SEQ ID No 263; SEQ ID No 264; SEQ ID No 265; SEQ ID No 266; SEQ ID No 267; SEQ ID No 268; SEQ ID No 269; SEQ ID No 270; SEQ ID No 271; SEQ ID No 272; SEQ ID No 273; SEQ ID No 274; SEQ ID No 275; SEQ ID No 276; SEQ ID No 277; SEQ ID No 278; SEQ ID No 279; SEQ ID No 280; SEQ ID No 281; SEQ ID No 282; SEQ ID No 283; SEQ ID No 284; SEQ ID No 285; SEQ ID No 286; SEQ ID No 287; SEQ ID No 288; SEQ ID No 289; SEQ ID No 290; SEQ ID No 291; SEQ ID No 292; SEQ ID No 293; SEQ ID No 294; SEQ ID No 295; SEQ ID No 296; SEQ ID No 297; SEQ ID No 298; SEQ ID No 299; SEQ ID No 300; SEQ ID No 301; SEQ ID No 302; SEQ ID No 303; SEQ ID No 304; SEQ ID No 305; SEQ ID No 306; SEQ ID No 307; SEQ ID No 308; SEQ ID No 309; SEQ ID No 310; SEQ ID No 311; SEQ ID No 312; SEQ ID No 313; SEQ ID No 314; SEQ ID No 315; SEQ ID No 316; SEQ ID No 317; SEQ ID No 318; SEQ ID No 319; SEQ ID No 320; SEQ ID No 321; SEQ ID No 322; SEQ ID No 323; SEQ ID No 324; SEQ ID No 325; SEQ ID No 326; SEQ ID No 327; SEQ ID No 328; SEQ ID No 329; SEQ ID No 330; SEQ ID No 331; SEQ ID No 332; SEQ ID No 333; SEQ ID No 334; SEQ ID No 335; SEQ ID No 336; SEQ ID No 337; SEQ ID No 338; SEQ ID No 339; SEQ ID No 340; SEQ ID No 341; SEQ ID No 342; SEQ ID No 343; SEQ ID No 344; SEQ ID No 345; SEQ ID No 346; SEQ ID No 347; SEQ ID No 348; and natural allelic variants thereof, such as more preferably SEQ ID No 340; SEQ ID No 341; SEQ ID No 342; SEQ ID No 343; SEQ ID No 344; SEQ ID No 345; SEQ ID No 346; SEQ ID No 347; SEQ ID No 348; and natural allelic variants thereof.

The term ‘natural allelic variants’ and the term ‘allelic variants’ encompasses both variants which although have a slightly different sequence (such as a homologue, fragment or variant), originate from the same chromosomal position, or the same position on an allelic chromosome, as the non-coding RNAs, and precursors thereof herein listed. The term ‘natural allelic variants’ and the term ‘allelic variants’ also encompasses mature non-coding RNAs, which may be differentially processed by the processing enzymes, as this may lead to variants of the same microRNAs having different lengths eg. shortened by 1 or 2 nucleotides, despite originating from the same allelic chromosome position.

The detection probe may be selected from the group consisting of: SEQ ID No. 114, SEQ ID No. 115, SEQ ID No. 116, SEQ ID No. 117, SEQ ID No. 118, SEQ ID No. 119, SEQ ID No. 120, SEQ ID No. 121, SEQ ID No. 122, SEQ ID No. 123, SEQ ID No. 124, SEQ ID No. 125, SEQ ID No. 126, SEQ ID No. 127, SEQ ID No. 128, SEQ ID No. 129, SEQ ID No. 130, SEQ ID No. 131, SEQ ID No. 132, SEQ ID No. 133, SEQ ID No. 134, SEQ ID No. 135, SEQ ID No. 136, SEQ ID No. 137, SEQ ID No. 138, SEQ ID No. 139, SEQ ID No. 140, SEQ ID No. 141, SEQ ID No. 142, SEQ ID No. 143, SEQ ID No. 144, SEQ ID No. 145, SEQ ID No. 147, SEQ ID No. 148, SEQ ID No. 149, SEQ ID No. 150, SEQ ID No. 151, SEQ ID No. 152, SEQ ID No. 153, SEQ ID No. 154, SEQ ID No. 155, SEQ ID No. 156, SEQ ID No. 157, SEQ ID No. 158, SEQ ID No. 159, SEQ ID No. 160, SEQ ID No. 161, SEQ ID No. 162, SEQ ID No. 163, SEQ ID No. 164, SEQ ID No. 165, SEQ ID No. 166, SEQ ID No. 167, SEQ ID No. 168, SEQ ID No. 169, SEQ ID No. 170, SEQ ID No. 171, SEQ ID No. 172, SEQ ID No. 173, SEQ ID No. 174, SEQ ID No. 175, SEQ ID No. 176, SEQ ID No. 177, SEQ ID No. 178, SEQ ID No. 179, SEQ ID No. 180, SEQ ID No. 181, SEQ ID No. 182, SEQ ID No. 183, SEQ ID No. 184, SEQ ID No. 185, SEQ ID No. 186, SEQ ID No. 187, SEQ ID No. 188, SEQ ID No. 189, SEQ ID No. 190, SEQ ID No. 191, SEQ ID No. 192, SEQ ID No. 193, SEQ ID No. 194, SEQ ID No. 195, SEQ ID No. 196, SEQ ID No. 197, SEQ ID No. 198, SEQ ID No. 199, SEQ ID No. 200, SEQ ID No. 201, SEQ ID No. 202, SEQ ID No. 203, SEQ ID No. 204, SEQ ID No. 205, SEQ ID No. 206, SEQ ID No. 207, SEQ ID No. 208, SEQ ID No. 209, SEQ ID No. 210, SEQ ID No. 211, SEQ ID No. 212, SEQ ID No. 213, SEQ ID No. 214, SEQ ID No. 215, SEQ ID No. 216, SEQ ID No. 217, SEQ ID No. 218; SEQ ID No 228; SEQ ID No 229; SEQ ID No 230; SEQ ID No 231; SEQ ID No 232; SEQ ID No 233; SEQ ID No 234; SEQ ID No 235; SEQ ID No 236; and variants, homologues and fragments thereof, preferably SEQ ID 175; SEQ ID 181; SEQ ID 120; SEQ ID 121; SEQ ID No 228; SEQ ID No 229; SEQ ID No 230; SEQ ID No 231; SEQ ID No 232; SEQ ID No 233; SEQ ID No 234; SEQ ID No 235; SEQ ID No 236; and variants, homologues and fragments thereof.

It will be recognised that a preferred design of the detection probes is to have a nucleotide analogue at every second, third or fourth position, although, independently, the first and/or last nucleobase may, in one embodiment be a nucleotide, such as a DNA or RNA unit, or in another embodiment the first and/or last nucleotide may be a nucleotide analogue. The following represent every two every three or every four designs:

XxXxXxXx (X) (x) (X) (x) (X) (x) (X) (x) (X) (x) (X) (x) (X) (x) (X) (x)
xXxXxXxX (x) (X) (x) (X) (x) (X) (x) (X) (x) (X) (x) (X) (x) (X) (x) (X)
xxXxxXxx (X) (x) (x) (X) (x) (x) (X) (x) (x) (X) (x) (x) (X) (x) (x) (X)
xXxxXxxX (x) (x) (X) (x) (x) (X) (x) (x) (X) (x) (x) (X) (x) (x) (X) (x)
XxxXxxXx (x) (X) (x) (x) (X) (x) (x) (X) (x) (x) (X) (x) (x) (X) (x) (x)
XxxxXxxx (X) (x) (x) (x) (X) (x) (x) (x) (X) (x) (x) (x) (X) (x) (x) (x)
xxxXxxxX (x) (x) (x) (X) (x) (x) (x) (X) (x) (x) (x) (X) (x) (x) (x) (X)
xxXxxxXx (x) (x) (X) (x) (x) (x) (X) (x) (x) (x) (X) (x) (x) (x) (X) (x)
xXxxxXxx (x) (X) (x) (x) (x) (X) (x) (x) (x) (X) (x) (x) (x) (X) (x) (x)

where x is a nucleotide such as DNA or RNA, and X is a nucleotide analogue, and the brakets reflect optional nucleobases representing a probe of between 8 and 24 nucleobases in length.

The detection probe may be selected from the group consisting of: tCcaTaaAgtAggAaaCacTaca; CtcAgtAatGgtAacGgt; AaaCtcAgtAatGgtAacGg; tccAtcAtcAaaAcaAatGgaGt; gaAcaGgtAgtCtgAacActGgg; tCtgTatCgtTccAatTt; GcgTgtCatCctTgcg; gaAtcTtgTccCgcAggt; gAacAggTagTctAaaCacTg; ggActTtgAggGccAgtt; aacCaaTgtGcaGacTacTgta; gGgcCtcCacTttGat; aTaaGgaTttTtaGggGcaTt; cAcaAacCatTatGtgCtgCta; gGcgAccCagAgg; acaGttCttCaaCtgGcaGctt; ctAccAtaGggTaaAacCact; aGtgCttCccTccAgag; aaCaaCcaGctAagAcaCtgCca; tgtAaaCcaTgaTgtGctGcta; ccAggTtcCacCccAgcAggc; ctGccTgtCtgTgcCtgCtgt; AaaGtgCatCccTctGga; acaCccCaaAatCgaAgcActTc; acaAagTtcTgtGatGcaCtga; gAacTgcCttTctCtcCa; agTgcTtcTtaCctCcaGa; AagTgcCccCatAgtTtgA; AacTgtTccCgcTgcTa; gcGgaActTagCcaCtgTgaa; GggGtaTttGacAaaCtgAca; gaGacCcaGtaGccAgaTgtAgct; cTtcCagTcgAggAtgTttAca; caAaaGagCccCcaGtt; tcCagTcaAggAtgTttAca; acTagActGtgAgcTccTc; ctCaaAggGctCctCag; acaAagTtcTgtGatGcaCtga; gGagAgcCagGagAa; gacGggTgcGatTtcTgtGtgAga; gCcaAtaTttCtgTgcTgcTa; gcAgaActTagCcaCtgTgaa; ctgGagGaaGggCccAgaGg; AccGacCgaCcgAtc; aGccTatGgaAttCagTtcTca; gGccCtgTgcTttGc; gGagCctCagTctAgt; tCcgTggTtcTacCctg; gCcaAtaTttCtgTgcTgcTa; aCtgTacAaaCtaCtaCctCa; gAaaCccAgcAgaCaaTgtAgct; aaGacGggAggAgag; gCtgAgaGtgTagGatGttTaca; aCcgAttTcaAatGgtGcta; acAggAttGagGggGggCcct; actAtaCaaCctCctAccTca; aaCtaTacAatCtaCtaCctCa; AagAacAgcCctCctCtg; gAacAgaTagTctAaaCacTggg; tCaaCatCagTctGatAagCta; ttTtcCcaTgcCctAtaCct; gcAagCccAgaCcgCaaAaag; aaTgaCacCtcCctGtga; aGagGttTccCgtGtaTg; gcAttAttAcCacGgtAcga; aCagCacAaaCtaCtaCctCa; gGaaAtcCctGgcAatGtgAt; gAaaAacGccCccTgg; cTgtTccTgcTgaActGagCca; ccaAtaTttAcgTgcTgcTa; tTcgCccTctCaaCccAgcTttt; caGacTccGgtGgaAtgAagGa; ccAtcAttAccCggCagTatTa; cAtcAttAccAggCagTatTaga; cacAagTtcGgaTctAcgGgtt; aaCcaTacAacCtaCtaCctCa; aaCcaCacAacCtaCtaCctCa; cCatCttTacCagAcaGtgTta; atcCaaTcaGttCctGatGcaGta; aaCtaTacAacCtaCtaCctCa; tcaCaaGttAggGtcTcaGgga; taGctGgtTgaAggGgaCcaa; GggActTtgTagGccAg; cTtcAgtTatCacAgtActg; tCctGggAaaActGga; cAtaCagCtaGatAacCaaAga; caCcaTtgTcaCacTccA; GaaAgaGacCggTtcActG; AgtGaaGacAcgGagC; acAggTtaAagGgtCtcAg; AgcTacAgtGctTcaTctCa; cCatCatCaaAacAaaTggAg; and variants, homologues and fragments therof, preferably cTtcAgtTatCacAgtActg; tCctGggAaaActGga; cAtaCagCtaGatAacCaaAga; caCcaTtgTcaCacTccA; GaaAgaGacCggTtcActG; AgtGaaGacAcgGagC; acAggTtaAagGgtCtcAg; AgcTacAgtGctTcaTctCa; cCatCatCaaAacAaaTggAg; and variants, homologues and fragments therof. (Residues in capitals are nucleotide analogues, such as LNA residues, residues in small letters are, preferably DNA residues, although in one embodiment they may be RNA residues (with U substituting for T) LNA cysteine residues are, in one embodiment, preferably methylated (such as with a 5-methyl substitution).

The terms ‘homologues’, ‘variants’ and ‘fragments’ in the context of ‘homologues, variants and fragments therof’ in relation to detection probe sequences and specific detection probes, refers to any sequence which has at least 8 consecutive nucleotide residues (or nucleotide analogues), such as at least 10 consecutive residues (or nucleotide analogues), such as at least 14 consecutive nucleotides (or nucleotide analogues), in common with at least one of the sequences, allowing for no more than 1 mismatch per 8 nucleotides (or nucleotide analogues), preferably with no more than 1 mismatch.

In one embodiment, the detection probe or probes are capable of selectively hybridizing to the precursor form of the non-coding RNA, but are not capable of selectively hybridizing to the mature form of the non-coding RNA. Suitable detection probes are routinely designed and made utilising the sequence information available at the miRBASE database (http://microrna.sanger.ac.uk/sequences/index.shtml). The database provides sequence listing of known mature siRNAs and their precursors, as well as the structural information relating to the precursor sequences which may be used for designing detection probes, which, for example will not specifically hybridize to the mature form, but only to the premature form of the non-coding RNA, e.g. by selecting a detection probe which at least partially hybridizes to the loop structure which is cleaved during miRNA processing. It should be noted that several mature miRNAs may originate from more than one precursor, hence by designing specific probes for a particular precursor, highly specific detection probes for use in the invention may be used.

The detection element of the detection probes according to the invention may be single or double labelled (e.g. by comprising a label at each end of the probe, or an internal position). In one aspect, the detection probe comprises two labels capable of interacting with each other to produce a signal or to modify a signal, such that a signal or a change in a signal may be detected when the probe hybridizes to a target sequence. A particular aspect is when the two labels comprise a quencher and a reporter molecule.

A particular detection aspect of the invention referred to as a “molecular beacon with a stem region” is when the recognition segment is flanked by first and second complementary hairpin-forming sequences which may anneal to form a hairpin. A reporter label is attached to the end of one complementary sequence and a quenching moiety is attached to the end of the other complementary sequence. The stem formed when the first and second complementary sequences are hybridized (i.e., when the probe recognition segment is not hybridized to its target) keeps these two labels in close proximity to each other, causing a signal produced by the reporter to be quenched by fluorescence resonance energy transfer (FRET). The proximity of the two labels is reduced when the probe is hybridized to a target sequence and the change in proximity produces a change in the interaction between the labels. Hybridization of the probe thus results in a signal (e.g. fluorescence) being produced by the reporter molecule, which can be detected and/or quantified.

Preferably, the detection probes of the invention are modified in order to increase the binding affinity of the probes for the target sequence by at least two-fold compared to probes of the same sequence without the modification, under the same conditions for hybridization or stringent hybridization conditions. The preferred modifications include, but are not limited to, inclusion of nucleobases, nucleosidic bases or nucleotides that have been modified by a chemical moiety or replaced by an analogue to increase the binding affinity. The preferred modifications may also include attachment of duplex-stabilizing agents e.g., such as minor-groove-binders (MGB) or intercalating nucleic acids (INA). Additionally, the preferred modifications may also include addition of non-discriminatory bases e.g., such as 5-nitroindole, which are capable of stabilizing duplex formation regardless of the nucleobase at the opposing position on the target strand. Finally, multi-probes composed of a non-sugar-phosphate backbone, e.g. such as PNA, that are capable of binding sequence specifically to a target sequence are also considered as a modification. All the different binding affinity-increasing modifications mentioned above will in the following be referred to as “the stabilizing modification(s)”, and the tagging probes and the detection probes will in the following also be referred to as “modified oligonucleotide”. More preferably the binding affinity of the modified oligonucleotide is at least about 3-fold, 4-fold, 5-fold, or 20-fold higher than the binding of a probe of the same sequence but without the stabilizing modification(s).

Most preferably, the stabilizing modification(s) is inclusion of one or more LNA nucleotide analogs. Probes from 8 to 30 nucleotides according to the invention may comprise from 1 to 8 stabilizing nucleotides, such as LNA nucleotides. When at least two LNA nucleotides are included, these may be consecutive or separated by one or more non-LNA nucleotides. In one aspect, LNA nucleotides are alpha-L-LNA and/or xylo LNA nucleotides as disclosed in PCT Publications No. WO 2000/66604 and WO 2000/56748.

In a preferable embodiment, each detection probe preferably comprises affinity enhancing nucleobase analogues, and wherein the recognition sequences exhibit a combination of high melting temperatures and low self-complementarity scores, said melting temperatures being the melting temperature of the duplex between the recognition sequence and its complementary DNA or RNA sequence.

This design provides for probes which are highly specific for their target sequences but which at the same time exhibit a very low risk of self-annealing (as evidenced by a low self-complementarity score)—self-annealing is, due to the presence of affinity enhancing nucleobases (such as LNA monomers) a problem which is more serious than when using conventional deoxyribonucleotide probes.

In one embodiment the recognition sequences exhibit a melting temperature (or a measure of melting temperature) corresponding to at least 5° C. higher than a melting temperature or a measure of melting temperature of the self-complementarity score under conditions where the probe hybridizes specifically to its complementary target sequence (alternatively, one can quantify the “risk of self-annealing” feature by requiring that the melting temperature of the probe-target duplex must be at least 5° C. higher than the melting temperature of duplexes between the probes or the probes internally).

In a preferred embodiment all of the detection probes include recognition sequences which exhibit a melting temperature or a measure of melting temperature corresponding to at least 5° C. higher than a melting temperature or a measure of melting temperature of the self-complementarity score under conditions where the probe hybridizes specifically to its complementary target sequence.

However, it is preferred that this temperature difference is higher, such as at least least 10° C., such as at least 15° C., at least 20° C., at least 25° C., at least 30° C., at least 35° C., at least 40° C., at least 45° C., and at least 50° C. higher than a melting temperature or measure of melting temperature of the self-complementarity score.

In one embodiment, the affinity-enhancing nucleobase analogues are regularly spaced between the nucleobases in said detection probes. One reason for this is that the time needed for adding each nucleobase or analogue during synthesis of the probes of the invention is dependent on whether or not a nucleobase analogue is added. By using the “regular spacing strategy” considerable production benefits are achieved. Specifically for LNA nucleobases, the required coupling times for incorporating LNA amidites during synthesis may exceed that required for incorporating DNA amidites. Hence, in cases involving simultaneous parallel synthesis of multiple oligonucleotides on the same instrument, it is advantageous if the nucleotide analogues such as LNA are spaced evenly in the same pattern as derived from the 3′-end, to allow reduced cumulative coupling times for the synthesis. The affinity enhancing nucleobase analogues are conveniently regularly spaced as every 2^{nd}, every 3^{rd}, every 4^{th} or every 5^{th} nucleobase in the recognition sequence, and preferably as every 3^{rd} nucleobase.

The presence of the affinity enhancing nucleobases in the recognition sequence preferably confers an increase in the binding affinity between a probe and its complementary target nucleotide sequence relative to the binding affinity exhibited by a corresponding probe, which only include nucleobases. Since LNA nucleobases/monomers have this ability, it is preferred that the affinity enhancing nucleobase analogues are LNA nucleobases.

In some embodiments, the 3′ and 5′ nucleobases are not substituted by affinity enhancing nucleobase analogues.

As detailed herein, one huge advantage of such probes for use in the method of the invention is their short lengths which surprisingly provides for high target specificity and advantages in detecting small RNAs and detecting nucleic acids in samples not normally suitable for hybridization detection strategies. It is, however, preferred that the probe comprising a recognition sequence is at least a 6-mer, such as at least a 7-mer, at least an 8-mer, at least a 9-mer, at least a 10-mer, at least an 11-mer, at least a 12-mer, at least a 13-mer, at least a 14-mer, at least a 15-mer, at least a 16-mer, at least a 17-mer, at least an 18-mer, at least a 19-mer, at least a 20-mer, at least a 21-mer, at least a 22-mer, at least a 23-mer, and at least a 24-mer. On the other hand, the recognition sequence is preferably at most a 25-mer, such as at most a 24-mer, at most a 23-mer, at most a 22-mer, at most a 21-mer, at most a 20-mer, at most a 19-mer, at most an 18-mer, at most a 17-mer, at most a 16-mer, at most a 15-mer, at most a 14-mer, at most a 13-mer, at most a 12-mer, at most an 11-mer, at most a 10-mer, at most a 9-mer, at most an 8-mer, at most a 7-mer, and at most a 6-mer.

The present invention provides oligonucleotide compositions and probe sequences for the use in detection, isolation, purification, amplification, identification, quantification, or capture of snRNAs, miRNAs, the target mRNAs of miRNAs, precursor RNAs, stem-loop precursor miRNAs, siRNAs, other non-coding RNAs, RNA-edited transcripts or alternative mRNA splice variants or single stranded DNA (e.g. viral DNA) characterized in that the probe sequences contain a number of nucleoside analogues.

In a preferred embodiment the number of nucleoside analogue corresponds to from 20 to 40% of the oligonucleotide of the invention.

In a preferred embodiment the probe sequences are substituted with a nucleoside analogue with regular spacing between the substitutions

In another preferred embodiment the probe sequences are substituted with a nucleoside analogue with irregular spacing between the substitutions

In a preferred embodiment the nucleoside analogue is LNA.

In a further preferred embodiment the detection probe sequences comprise a photochemically active group, a thermochemically active group, a chelating group, a reporter group, or a ligand that facilitates the direct of indirect detection of the probe or the immobilization of the oligonucleotide probe onto a solid support.

In a further preferred embodiment:

(a) the photochemically active group, the thermochemically active group, the chelating group, the reporter group, or the ligand includes a spacer (K), said spacer comprising a chemically cleavable group; or

(b) the photochemically active group, the thermochemically active group, the chelating group, the reporter group, or the ligand is attached via the biradical of at least one of the LNA(s) of the oligonucleotide.

Especially preferred detection probes of the invention are those that include the LNA containing recognition sequences set forth in tables A-K, 1, 3 and 15-I herein.

Methods for defining and preparing probes and probe collections are disclosed in PCT/DK2005/000838.

The Target

The term the ‘target’ ‘or complementary target’ refers to a non-coding polynucleotide sequence associated with cancer, preferably an RNA sequence such as a snRNA, miRNA, siRNA, or precursor sequence thereof, or a sequence derived therefrom which retains the sequence information present in the non-coding RNA sequence.

In one embodiment, the target may be selected from any one of SEQ ID 1, SEQ ID 2, SEQ ID 3, SEQ ID 4, SEQ ID 5, SEQ ID 6, SEQ ID 7, SEQ ID 8, SEQ ID 9, SEQ ID 10, SEQ ID 11, SEQ ID 12, SEQ ID 13, SEQ ID 14, SEQ ID 15, SEQ ID 16, SEQ ID 17, SEQ ID 18, SEQ ID 19, SEQ ID 20, SEQ ID 21, SEQ ID 22, SEQ ID 23, SEQ ID 24, SEQ ID 25, SEQ ID 26, SEQ ID 27, SEQ ID 28, SEQ ID 29, SEQ ID 30, SEQ ID 31, SEQ ID 32, SEQ ID 33, SEQ ID 34, SEQ ID 35, SEQ ID 36, SEQ ID 37, SEQ ID 38, SEQ ID 39, SEQ ID 40, SEQ ID 41, SEQ ID 42, SEQ ID 43, SEQ ID 44, SEQ ID 45, SEQ ID 46, SEQ ID 47, SEQ ID 48, SEQ ID 49, SEQ ID 50, SEQ ID 51, SEQ ID 52, SEQ ID 53, SEQ ID 54, SEQ ID 55, SEQ ID 56, SEQ ID 57, SEQ ID 58, SEQ ID 59, SEQ ID 60, SEQ ID 61, SEQ ID 62, SEQ ID 63, SEQ ID 64, SEQ ID 65, SEQ ID 66, SEQ ID 67, SEQ ID 68, SEQ ID 69, SEQ ID 70, SEQ ID 71, SEQ ID 72, SEQ ID 73, SEQ ID 74, SEQ ID 75, SEQ ID 76, SEQ ID 77, SEQ ID 78, SEQ ID 79, SEQ ID 80, SEQ ID 81, SEQ ID 82, SEQ ID 83, SEQ ID 84, SEQ ID 85, SEQ ID 86, SEQ ID 87, SEQ ID 88, SEQ ID 89, SEQ ID 90, SEQ ID 91, SEQ ID 92, SEQ ID 93, SEQ ID 94, SEQ ID 95, SEQ ID 96, SEQ ID 97, SEQ ID 98, SEQ ID 99, SEQ ID 100, SEQ ID 101, SEQ ID 102, SEQ ID 103, SEQ ID 104, SEQ ID 105, SEQ ID 106, SEQ ID 107, SEQ ID 108, SEQ ID 109, SEQ ID 110, SEQ ID 111, SEQ ID 112, SEQ ID No 113; SEQ ID No. 32; SEQ ID No 219; SEQ ID No 220; SEQ ID No 221; SEQ ID No 222; SEQ ID No 223; SEQ ID No 224; SEQ ID No 225; SEQ ID No 226; SEQ ID No 227; SEQ ID No 349; SEQ ID No 350; SEQ ID No 351; SEQ ID No 352; SEQ ID No 353; SEQ ID No 354; SEQ ID No 355; SEQ ID No 356; SEQ ID No 357; SEQ ID No 358; SEQ ID No 359; SEQ ID No 360; SEQ ID No 361; SEQ ID No 362; SEQ ID No 363; SEQ ID No 364; SEQ ID No 365; SEQ ID No 366; and SEQ ID No 367; and allelic variants thereof.

Preferably the target is selected from the group consisting of: SEQ ID No. 4; SEQ ID No. 72; SEQ ID No. 36; SEQ ID No. 29; SEQ ID No. 44; SEQ ID No. 65; SEQ ID No. 76; SEQ ID No. 12; SEQ ID No. 28; SEQ ID No. 83; SEQ ID No. 52; SEQ ID No. 75; SEQ ID No. 91; SEQ ID No. 9; SEQ ID No. 85; SEQ ID No. 92; SEQ ID No. 26; SEQ ID No. 14; SEQ ID No. 46; SEQ ID No. 39; SEQ ID No. 69; SEQ ID No. 66; SEQ ID No. 6; SEQ ID No. 64; SEQ ID No. 84; SEQ ID No. 93; SEQ ID No. 54; SEQ ID No. 24; SEQ ID No. 42; SEQ ID No. 94; SEQ ID No. 95; SEQ ID No. 18; SEQ ID No. 90; SEQ ID No. 87; SEQ ID No. 6; SEQ ID No. 82; SEQ ID No. 23; SEQ ID No. 55; SEQ ID No. 57; SEQ ID No. 33; SEQ ID No. 88; SEQ ID No. 37; SEQ ID No. 96; SEQ ID No. 97; SEQ ID No. 85; SEQ ID No. 55; SEQ ID No. 53; SEQ ID No. 58; SEQ ID No. 68; SEQ ID No. 59; SEQ ID No. 73; SEQ ID No. 41; SEQ ID No. 19; SEQ ID No. 67; SEQ ID No. 89; SEQ ID No. 76; SEQ ID No. 45; SEQ ID No. 63; SEQ ID No. 25; SEQ ID No. 62; SEQ ID No. 21; SEQ ID No. 78; SEQ ID No. 13; SEQ ID No. 50; SEQ ID No. 3; SEQ ID No. 27; SEQ ID No. 10; SEQ ID No. 38; SEQ ID No. 47 (V. PREF); SEQ ID No. 77; SEQ ID No. 51; SEQ ID No. 11; SEQ ID No. 30; SEQ ID No. 43; SEQ ID No. 22; SEQ ID No. 1; SEQ ID No. 40; SEQ ID No. 48; SEQ ID No 111; SEQ ID No 112; SEQ ID No 113; and SEQ ID No. 32; SEQ ID No 219; SEQ ID No 220; SEQ ID No 221; SEQ ID No 222; SEQ ID No 223; SEQ ID No 224; SEQ ID No 225; SEQ ID No 226; SEQ ID No 227; SEQ ID No 349; SEQ ID No 350; SEQ ID No 351; SEQ ID No 352; SEQ ID No 353; SEQ ID No 354; SEQ ID No 355; SEQ ID No 356; SEQ ID No 357; SEQ ID No 358; SEQ ID No 359; SEQ ID No 360; SEQ ID No 361; SEQ ID No 362; SEQ ID No 363; SEQ ID No 364; SEQ ID No 365; SEQ ID No 366; and SEQ ID No 367; and allelic variants thereof.

Preferably the target is a human miRNA or snRNA or precursor thereof.

In one specific embodiment the target is a snRNA, such as the human U6 snRNA.

The Signal

In one embodiment the target is labelled with a signal. In this respect the population of nucleic acids is labelled with a signal which can be detected. The hybridization of the target molecules to the detection probe, which may be fixed to a solid surface, and subsequent removal of the remaining nucleic acids from the population, and therefore allows the determination of the level of signal from those labelled target which is bound to the detection probe. This may be appropriate when screening immobilised probes, such as arrays of detection probes.

In one embodiment the detection probe is labelled with a signal. This may be appropriate, for example, when performing in situ hybridization and northern blotting, where the population of nucleic acids is immobilised.

It is also envisaged that both population of nucleic acids and detection probes are labelled. For example they may be labelled with fluorescent probes, such as pairs of FRET probes (Fluorescence resonance energy transfer), so that when hybridization occurs, the FRET pair is formed, which causes a shift in the wavelength of fluorescent light emited. It is also envisaged that pairs of detection probes may be used designed to hybridize to adjacent regions of the target molecule, and each detection probe carrying one half of a FRET pair, so that when the probes hybridize to their respective positions on the target, the FRET pair is formed, allowing the shift in fluorescence to be detected.

Therefore, it is also envisaged that neither the population of nucleic acid molecules or the detection probe need be immobilised.

Once the appropriate target RNA sequences have been selected, probes, such as the preferred LNA substituted detection probes are preferably chemically synthesized using commercially available methods and equipment as described in the art (Tetrahedron 54: 3607-30, 1998). For example, the solid phase phosphoramidite method can be used to produce short LNA probes (Caruthers, et al., Cold Spring Harbor Symp. Quant. Biol. 47:411-418, 1982, Adams, et al., J. Am. Chem. Soc. 105: 661 (1983).

Detection probes, such as LNA-containing-probes, can be labelled during synthesis. The flexibility of the phosphoramidite synthesis approach furthermore facilitates the easy production of detection probes carrying all commercially available linkers, fluorophores and labelling-molecules available for this standard chemistry. Detection probes, such as LNA-modified probes, may also be labelled by enzymatic reactions e.g. by kinasing using T4 polynucleotide kinase and gamma-³²P-ATP or by using terminal deoxynucleotidyl transferase (TDT) and any given digoxygenin-conjugated nucleotide triphosphate (dNTP) or dideoxynucleotide triphosphate (ddNTP).

Detection probes according to the invention can comprise single labels or a plurality of labels. In one aspect, the plurality of labels comprise a pair of labels which interact with each other either to produce a signal or to produce a change in a signal when hybridization of the detection probe to a target sequence occurs.

In another aspect, the detection probe comprises a fluorophore moiety and a quencher moiety, positioned in such a way that the hybridized state of the probe can be distinguished from the unhybridized state of the probe by an increase in the fluorescent signal from the nucleotide. In one aspect, the detection probe comprises, in addition to the recognition element, first and second complementary sequences, which specifically hybridize to each other, when the probe is not hybridized to a recognition sequence in a target molecule, bringing the quencher molecule in sufficient proximity to said reporter molecule to quench fluorescence of the reporter molecule. Hybridization of the target molecule distances the quencher from the reporter molecule and results in a signal, which is proportional to the amount of hybridization.

In the present context, the term “label” means a reporter group, which is detectable either by itself or as a part of a detection series. Examples of functional parts of reporter groups are biotin, digoxigenin, fluorescent groups (groups which are able to absorb electromagnetic radiation, e.g. light or X-rays, of a certain wavelength, and which subsequently reemits the energy absorbed as radiation of longer wavelength; illustrative examples are DANSYL (5-dimethylamino)-1-naphthalenesulfonyl), DOXYL (N-oxyl-4,4-dimethyloxazolidine), PROXYL (N-oxyl-2,2,5,5-tetramethylpyrrolidine), TEMPO (N-oxyl-2,2,6,6-tetramethylpiperidine), dinitrophenyl, acridines, coumarins, Cy3 and Cy5 (trademarks for Biological Detection Systems, Inc.), erythrosine, coumaric acid, umbelliferone, Texas red, rhodamine, tetramethyl rhodamine, Rox, 7-nitrobenzo-2-oxa-1-diazole (NBD), pyrene, fluorescein, Europium, Ruthenium, Samarium, and other rare earth metals), radio isotopic labels, chemiluminescence labels (labels that are detectable via the emission of light during a chemical reaction), spin labels (a free radical (e.g. substituted organic nitroxides) or other paramagnetic probes (e.g. Cu²⁺, Mg²⁺) bound to a biological molecule being detectable by the use of electron spin resonance spectroscopy). Especially interesting examples are biotin, fluorescein, Texas Red, rhodamine, dinitrophenyl, digoxigenin, Ruthenium, Europium, Cy5, Cy3, etc.

Control Detection Probes

It is preferably in the method according to the invention that in addition to the detection probe for the target in question, at least one further detection probe is used, where the at least one further detection probe is capable of hybridizing to a control nucleic acid (control target) present in said population of nucleic acids (such as the RNA fraction). The control nucleic acid is not the same as the target in question.

In one embodiment, the at least one further detection probe may be derived from or is capable of selectively hybridizing with a molecule selected from the group consisting of: a pre-miRNA molecule; a pre-siRNA molecule; and a pre-piRNA molecule.

In another embodiment, the at least one further detection probe may be derived from or is capable of selectively hybridizing with a molecule selected from the group consisting of a mature miRNA, a mature siRNA, a mature piRNA and a snRNA.

In a preferred embodiment, the at least one further detection probe may be derived from or is capable of selectively hybridizing with a snRNA.

A further type of detection probe, which may be used with as a detection probe control and/or as a detection probe, is one which is capable of hybridizing to the loop region of an immature miRNA, siRNA or piRNA. Recent research has shown that the processing of pre-microRNAs to mature microRNAs may be controlled in a cell specific manner (Obernosterer et al). In this respect the ratio between the immature and mature form can give valuable information which may be used to characterize the cancer test sample.

Detection Probes to Precursor Non-Coding RNAs

The present invention provides for detection probes for the detection of non coding RNA precursors, such as pre-miRNAs, pre-siRNAs and pre-piRNAs, and their targets. miRNAs are transcribed as mono- or poly-cistronic, long, primary precursor transcripts (pri-miRNAs) that are cleaved into ˜70-nt precursor hairpins, known as microRNA precursors (pre-miRNAs), by the nuclear RNase III-like enzyme Drosha (Lee et al., Nature 425:415-419, 2003). MicroRNA precursors (pre-miRNAs) form hairpins having a loop region and a stem region containing a duplex of the opposite ends of the RNA strand. Subsequently pre-miRNA hairpins are exported to the cytoplasm by Exportin-5 (Yi et al., Genes & Dev., 17:3011-3016, 2003; Bohnsack et al., RNA, 10:185-191, 2004), where they are processed by a second RNase III-like enzyme, termed Dicer, into ˜22-nt duplexes (Bernstein et al., Nature 409:363-366, 2001), followed by the asymmetric assembly of one of the two strands into a functional miRNP or miRISC (Khvorova et al., Cell 115:209-216, 2003). miRNAs can recognize regulatory targets while part of the miRNP complex and inhibit protein translation. Alternatively, the active RISC complex is guided to degrade the specific target mRNAs (Upardi et al., Cell 107:297-307, 2001; Zhang et al., EMBO J. 21:5875-5885, 2002; Nykänen et al., Cell 107:309-321, 2001). There are several similarities between miRNP and the RNA-induced silencing complex, miRISC, including similar sizes and both containing RNA helicase and the PPD proteins. It has therefore been proposed that miRNP and miRISC are the same RNP with multiple functions (Ke et al., Curr. Opin. Chem. Biol. 7:516-523, 2003).

Most reports in the literature have described the processing of miRNAs to be complete, suggesting that intermediates like pri-miRNA and pre-miRNA rarely accumulate in cells and tissues. However, previous studies describing miRNA profiles of cells and tissues have only investigated size-fractionated RNAs pools. Consequently the presence of larger miRNA precursors has been overlooked.

Alterations in miRNA biogenesis resulting in different levels of mature miRNAs and their miRNA precursors could illuminate the mechanisms underlying many disease processes. For example, the 26 miRNA precursors were equally expressed in non-cancerous and cancerous colorectal tissues from patients, whereas the expression of mature human miR-143 and miR-145 was greatly reduced in cancer tissues compared with non-cancer tissues, suggesting altered processing for specific miRNAs in human disease (Michael et al., Mol. Cancer Res. 1:882-891, 2003).

Connections between miRNAs, their precursors, and human diseases will only strengthen in parallel with the knowledge of miRNA, their precursors, and the gene networks that they control. Moreover, the understanding of the regulation of RNA-mediated gene expression is leading to the development of novel therapeutic approaches that will be likely to revolutionize the practice of medicine (Nelson et al., TIBS 28:534-540, 2003).

siRNAs and piRNAs are considered to undergo a similar processing from precursor molecules.

To this end, the invention provides oligonucleotide probes for precursors of non-coding RNAS, such as miRNA precursors, siRNA precursors, and piRNA precursors.

The detection probes for precursors may be a detection probe that hybridizes to a non-coding RNA precursor molecule, wherein at least part of said probe hybridizes to a portion of said precursor not present in the corresponding mature non coding RNA, e.g. the loop region.

Such oligonucleotide probes include a sequence complementary to the desired RNA sequence and preferably a substitution with nucleotide analogues, preferably high-affinity nucleotide analogues, e.g., LNA, to increase their sensitivity and specificity over conventional oligonucleotides, such as DNA oligonucleotides, for hybridization to the desired RNA sequences.

An exemplary oligonucleotide probe includes a plurality of nucleotide analogue monomers and hybridizes to a miRNA precursor. Desirably, the nucleotide analogue is LNA, wherein the LNA may be oxy-LNA, preferably beta-D-oxy-LNA, monomers. Desirably, the oligonucleotide probe will hybridize to part of the loop sequence of a miRNA precursor, e.g., to 5 nucleotides of the miRNA precursor loop sequence or to the center of the miRNA precursor loop sequence. In other embodiments, the oligonucleotide probe will hybridize to part of the stem sequence of a miRNA precursor.

The invention also features a method of measuring relative amounts of non coding RNAs, such as miRNa, piRNA and siRNA, and their precursors, such as pre-miRNAs, pre-siRNAs and pre-piRNAs-

This may be achieved by using a detection probe pair which comprises of i) a first detection probe that hybridizes to a non-coding RNA precursor molecule, wherein at least part of said probe hybridizes to a portion of said precursor not present in the corresponding mature non coding RNA, and ii) a further detection probe that hybridizes to the mature non-coding RNA, but will not hybridize to the precursor non-coding RNA, e.g. by designing the detection probe to hybridize to the sequence which flanks the stem loop splice site of the precursor molecule. The ratio of signal of hybridization thereby provides data which can provide said characterisation of said breast cancer.

In one embodiment, the comparison is made by contacting a first probe that hybridizes to the mature noncoding RNA, such as mature miRNA, with the sample under a first condition that also allows the corresponding non-coding RNA precursor, such as miRNA precursor to hybridize; contacting the first probe or a second probe that hybridizes to mature non-coding RNA with the sample under a second condition that does not allow corresponding miRNA precursor to hybridize; comparing the amounts of the probes hybridized under the two conditions wherein the reduction in amount hybridized under the second condition compared to the first condition is indicative of the amount of the miRNA precursor in the sample.

Furthermore, the invention features a kit including a probe of the invention (or a detection probe pair according to the invention) and packaging and/or labeling indicative of the non-coding RNA and/or non-coding precursor (e.g. miRNA precursor), to which the probe (or probe pair) hybridizes and conditions under which the hybridization occurs. The kit provides for the isolation, purification, amplification, detection, identification, quantification, or capture of natural or synthetic nucleic acids. The probes are preferably immobilized onto a solid support, e.g., such as a bead or an array.

The invention also features a method of treating a disease or condition in a living organism using any combination of the probes and methods of the invention.

The invention further features a method of comparing relative amounts of miRNA and miRNA precursor in a sample by contacting the sample with a first probe that hybridizes to miRNA precursor and a second probe that hybridizes to miRNA; and detecting the amount of one or more signals indicative of the relative amounts of miRNA and miRNA precursor.

The invention also features a method of measuring relative amounts of miRNA and miRNA precursor in a sample by contacting a first probe that hybridizes to miRNA with the sample under conditions that also allow miRNA precursor to hybridize; contacting the first probe or a second probe that hybridizes to miRNA with the sample under conditions that do not allow miRNA precursor to hybridize; comparing the amounts of the probes hybridized under the two conditions wherein the reduction in amount hybridized under the second condition compared to the first condition is indicative of the amount of miRNA precursor in the sample.

The invention also features methods of using the probes of the invention as components of Northern blots, in situ hybridization, arrays, and various forms of PCR analysis including PCR, RT-PCR, and qPCR.

Any probe of the invention may be used in performing any method of the invention. For example, any method of the invention may involve probes having labels. Furthermore, any method of the invention may also involve contacting a probe with miRNA precursor that is endogenously or exogenously produced. Such contacting may occur in vitro or in vivo, e.g., such as in the body of an animal, or within or without a cell, which may or may not naturally express the miRNA precursor.

Also, primarily with respect to miRNA precursors, nucleotide analogue containing probes, polynucleotides, and oligonucleotides are broadly applicable to antisense uses. To this end, the present invention provides a method for detection and functional analysis of non-coding antisense RNAs, as well as a method for detecting the overlapping regions between sense-antisense transcriptional units.

The oligonucleotide probes of invention are also useful for detecting, testing, diagnosing or quantifying miRNA precursors and their targets implicated in or connected to human disease, e.g., analyzing human samples for cancer diagnosis.

For example, pre-mir-138-2 is ubiquitously expressed, unlike its mature miRNA derivative. The presence of an unprocessed miRNA precursor in most tissues of the organism suggests miRNA precursors as possible diagnostic targets. We envision that miRNA precursor processing could be a more general feature of the regulation of miRNA expression and be used to identify underlying disease processes. One could also imagine that the unprocessed miRNA precursors might play a different role in the cell, irrespective of the function of the mature miRNA, providing further insights into underlying disease processes.

Imperfect processing of miRNA precursors to mature miRNA as detected by sample hybridization to oligonucleotide probes may provide diagnostic or prognostic information. Specifically, the ratio between levels of mature and precursor transcripts of a given miRNA may hold prognostic or diagnostic information. Furthermore, specific spatial expression patterns of mature miRNA compared to miRNA precursor may likewise hold prognostic or diagnostic information. In addition, performing in situ hybridization using mature miRNA and/or miRNA precursor specific oligonucleotide probes could also detect abnormal expression levels. LNA-containing probes are particularly well-suited for these purposes.

The present invention enables discrimination between different polynucleotide transcripts and detects each variant in a nucleic acid sample, such as a sample derived from a patient, e.g., addressing the spatiotemporal expression patterns by RNA in situ hybridization. The methods are thus applicable to tissue culture animal cells, animal cells (e.g., blood, serum, plasma, reticulocytes, lymphocytes, urine, bone marrow tissue, cerebrospinal fluid or any product prepared from blood or lymph) or any type of tissue biopsy (e.g., a muscle biopsy, a liver biopsy, a kidney biopsy, a bladder biopsy, a bone biopsy, a cartilage biopsy, a skin biopsy, a pancreas biopsy, a biopsy of the intestinal tract, a thymus biopsy, a mammae biopsy, a uterus biopsy, a testicular biopsy, an eye biopsy or a brain biopsy, e.g., homogenized in lysis buffer), archival tissue nucleic acids such as formalin fixated paraffine embedded sections of the tissue, and the like.

pre-mir-138-1 and pre-mir-138-2 and their shared mature miRNA derivative mir-138 differ in their expression levels across various tissues as detected by oligonucleotide probes. The differential expression of pre-mir-138-1 and pre-mir-138-2 and their derived mature miRNA mir-138. pre-mir-138-2 is expressed in all tissues, and mir-138 is expressed in a tissue-specific manner. Furthermore, the experiments suggest that an inhibitory factor is responsible for tissue-specific processing of pre-mir-138-2 into mir-138 and that this inhibitory factor is specific for certain miRNA precursors. This inhibitory factor acting on pre-138-2 may be capable of distinguishing pre-mir-138-1 from pre-mir-138-2 as well. pre-mir-138-1 and pre-mir-138-2 have different sequences, particularly in the loop region, and thus the inhibitory factor may be capable of recognizing these sequence differences to achieve such specificity. It is hypothesized that recognition by an inhibitory factor is dependent on the differences in the loop sequence, e.g., the size of the loop sequence, between pre-mir-138-1 and pre-mir-138-2. It is therefore possible that an oligonucleotide probe capable of hybridizing specifically to the sequences that are different between pre-mir-138-1 and pre-mir-138-2, e.g. in the loop region, could be utilized to block the inhibitory effect of the inhibitory factor, thereby allowing the pre-mir-138-2 to be processed.

Signal Data

The signal data obtained from the hybridization experiment may be a quantative measurement of the level of signal detected.

The signal data obtained from the hybridization experiment may be a qualitative measurement of the level of signal detected.

For example, in the case of non-coding RNAs whose presence or absence is indicative of the presence or absence of a feature of the cancer, the detection of signal, i.e. positive signal data or negative signal data may be a direct indication of the feature in question.

In one embodiment the signal data may be used to obtain a ratio of the signals obtained between the test sample and a control sample, or a matrix between the signal between the control sample and more than one of the controls as herein provided. The ratio or matrix being indicative of the feature in question.

The signal data from numerous hybridizations, for example arrays of a collection of detection probes may provide signals from hybridizations with several different targets, and it is the differential pattern of targets which allows for one or more of the features in question to be determined. Typically, the determination of previously characterized cancers can provide a dataset which can subsequently be used for comparison with data obtained from samples from a patient, thereby allowing determination of the features.

Therefore, in one embodiment, the method of the invention comprises the hybridization of the test sample and one or more control samples to both i) one or more target detection probes, such as a collection of detection probes, which may be in the form as listed above, such as an array such as a microarray, and ii) one or more control detection probes, such as

at least one normalizing control probe and at least one mRNA marker control probe,

or

at least one normalizing control probe and at least one DNA marker control probe and optionally at least one mRNA marker control probe.

or

at least one normalizing control probe and at least one immature noncoding RNA, selected from immature miRNA, immature siRNA and immature piRNA, and optionally at least one DNA marker control probe and optionally at least one mRNA marker control probe.

Collection of Probes of the Invention

In one embodiment a collection of probes according to the present invention comprises at least 10 detection probes, 15 detection probes, such as at least 20, at least 25, at least 50, at least 75, at least 100, at least 200, at least 500, at least 1000, and at least 2000 members.

In a preferred embodiment the collection of probes comprise at least one probe which is complementary to a region of a (target) snRNA.

The collection of detection probes may comprise a majority of detection probes to the target as compared to the control probes.

In one embodiment, at least 10%, such as at least 20%, such as at least 30%, such as at least 40%, such as at least 50%, such at least 60%, such as at least 70%, such as at least 80%, such as at least 90 or 95% of the detection probes in the collection of detection probes may be capable of hybridizing to the respective population of target molecules (as opposed to control-targets).

The collection of detection probes prepferably comprises at least one control detection probe, and may comprise a collection of control detection probes.

In one embodiment, the collection of probes according to the present invention consists of no more than 500 detection probes, such as no more than 200 detection probes, such as no more than 100 detection probes, such as no more than 75 detection probes, such as no more than 50 detection probes, such as no more that 50 detection probes, such as no more than 25 detection probes, such as no more than 20 detection probes.

In one embodiment, the collection of probes according to the present invention has between 3 and 100 detection probes, such as between 5 and 50 detection probes, such as between 10 and 25 detection probes.

In one embodiment, the collection of probes of the invention is capable of specifically detecting all or substantially all members of the transcriptome of an organism.

In another embodiment, the collection of probes is capable of specifically detecting all small non-coding RNAs of an organism, such as all miRNAs, piRNAs, snRNAs and/or siRNAs.

In a preferred embodiment, the collection of probes is capable of specifically detecting a subset of non-coding RNAs, preferably a subset which has been selected for their ability to act as markers for at least one type of cancer, and preferably appropriate control probes or collection of control probes.

In one embodiment, the affinity-enhancing nucleobase analogues, such as LNA nucleobases, are regularly spaced between the nucleobases in at least 80% of the members of said collection, such as in at least 90% or at least 95% of said collection (in one embodiment, all members of the collection contains regularly spaced affinity-enhancing nucleobase analogues). It is recognized that in addition to the regularly spaced nucleotide analogues the detection probes may, in one embodiment, have additional 5′ and/or 3′ nucleobases which may be for example DNA nucleobases.

In one embodiment of the the collection of probes, all members contain affinity enhancing nucleobase analogues with the same regular spacing in the recognition sequences.

Also for production purposes, it is an advantage that a majority of the probes in a collection are of the same length. In preferred embodiments, the collection of probes of the invention is one wherein at least 80% of the members comprise recognition sequences of the same length, such as at least 90% or at least 95%.

As discussed above, it is advantageous, in order to avoid self-annealing, that at least one of the nucleobases in the recognition sequence is substituted with its corresponding selectively binding complementary (SBC) nucleobase.

Typically, the nucleobases in the sequence are selected from ribonucleotides and deoxyribonucleotides, preferably deoxyribonucleotides. It is preferred that the recognition sequence consists of affinity enhancing nucleobase analogues together with either ribonucleotides or deoxyribonucleotides.

In certain embodiments, each member of a collection is covalently bonded to a solid support. Such a solid support may be selected from a bead, a microarray, a chip, a strip, a chromatographic matrix, a microtiter plate, a fiber or any other convenient solid support generally accepted in the art in order to facilitate the exercise of the methods discussed generally and specifically

The collection may be so constituted that at least 90% (such as at least 95%) of the recognition sequences exhibit a melting temperature or a measure of melting temperature corresponding to at least 5° C. higher than a melting temperature or a measure of melting temperature of the self-complementarity score under conditions where the probe hybridizes specifically to its complementary target sequence (or that at least the same percentages of probes exhibit a melting temperature of the probe-target duplex of at least 5° C. more than the melting temperature of duplexes between the probes or the probes internally).

As also detailed herein, each detection probe in a collection of the invention may include a detection moiety and/or a ligand, optionally placed in the recognition sequence but also placed outside the recognition sequence. The detection probe may thus include a photochemically active group, a thermochemically active group, a chelating group, a reporter group, or a ligand that facilitates the direct of indirect detection of the probe or the immobilisation of the oligonucleotide probe onto a solid support.

Methods/Uses of Probes and Probe Collections

Preferred methods/uses include: Specific isolation, purification, amplification, detection, identification, quantification, inhibition or capture of a target nucleotide sequence in a sample, wherein said target nucleotide sequence is associated with cancer, such as breast cancer, by contacting said sample with a member of a collection of probes or a probe defined herein under conditions that facilitate hybridization between said member/probe and said target nucleotide sequence. Since the probes are typically shorter than the complete molecule wherein they form part, the inventive methods/uses include isolation, purification, amplification, detection, identification, quantification, inhibition or capture of a molecule comprising the target nucleotide sequence.

Typically, the molecule which is isolated, purified, amplified, detected, identified, quantified, inhibited or captured is a small, non-coding RNA, e.g. a snRNA or and miRNA such as a mature miRNA.

In one embodiment, the small, non-coding RNA has a length of at most 30 residues, such as at most 29, 28, 27, 26, 25, 24, 23, 22, 21, 20, 19, or 18 residues. The small non-coding RNA typically also has a length of at least 15 residues, such as at least 16, 17, 18, 19, 20, 21, 22, 23, 24, 25, 26, 27, 28, 29 or 30 residues.

As detailed in PCT/DK2005/000838, the specific hybridization between the short probes of the present invention to miRNA and the fact that miRNA can be mapped to various tissue origins, allows for an embodiment of the uses/methods of the present invention comprising identification of the primary site of metastatic tumors of unknown origin.

As also detailed in PCT/DK2005/000838, the short, but highly specific probes of the present invention allow hybridization assays to be performed on fixated embedded tissue sections, such as formalin fixated paraffine embedded sections. Hence, an embodiment of the uses/methods of the present invention are those where the molecule, which is isolated, purified, amplified, detected, identified, quantified, inhibited or captured, is DNA (single stranded such as viral DNA) or RNA present in a fixated, embedded sample such as a formalin fixated paraffine embedded sample.

The detection probes herein disclosed may also be used for detection and assessment of expression patterns for naturally occurring single stranded nucleic acids such as snRNAs, miRNAs, their target mRNAs, stem-loop precursor miRNAs, siRNAs, piRNAs, other non-coding RNAs, RNA-edited transcripts or alternative mRNA splice variants by RNA in-situ hybridization, dot blot hybridization, reverse dot blot hybridization, or in Northern blot analysis or expression profiling by microarrays.

In one embodiment the hybridization occurs as an in situ hybridization of a test sample, such as a biopsy, taken from a patient during an operation. The use of in situ hybridization is preferred when the two dimensional location of the target molecule is to be used in determining the feature of the cancer. For example, cancers are often made up of vascular cells, connective tissue etc as well as cancerous cells, the use of in situ hybridization therefore allows a morphological distinction to be made between hybridization in non cancer cells and cancer cells within a sample. Typically the in situ hybridization is performed using only a few detection probes, such as between 1 and three detection probes, such as two detection probes. One or two of the detection probes may be control probes. The in situ hybridization may be performed during or subsequent to a method of therapy such as surgery for removal or biopsy of a cancer.

The detection probes herein disclosed may also be used for antisense-based intervention, targeted against tumorigenic single stranded nucleic acids such as snRNAs, miRNAs, their target mRNAs, stem-loop precursor miRNAs, siRNAs, piRNAs, other non-coding RNAs, RNA-edited transcripts or alternative mRNA splice variants or viral DNA in vivo in plants or animals, such as human, mouse, rat, by inhibiting their mode of action, e.g. the binding of mature miRNAs to their cognate target mRNAs.

Further embodiments includes the use of the detection probe as an aptamer in molecular diagnostics or (b) as an aptamer in RNA mediated catalytic processes or (c) as an aptamer in specific binding of antibiotics, drugs, amino acids, peptides, structural proteins, protein receptors, protein enzymes, saccharides, polysaccharides, biological cofactors, nucleic acids, or triphosphates or (d) as an aptamer in the separation of enantiomers from racemic mixtures by stereospecific binding or (e) for labelling cells or (f) to hybridize to non-protein coding cellular RNAs, such as tRNA, rRNA, snRNA and scRNA, in vivo or in vitro or (g) to hybridize to non-protein coding cellular RNAs, such as tRNA, rRNA, snRNA and scRNA, in vivo or in vitro or (h) in the construction of Taqman probes or Molecular Beacons.

The present invention also provides a kit for the isolation, purification, amplification, detection, identification, quantification, or capture of nucleic acids, wherein said nucleic acids are associated with cancer, such as the cancers herein disclosed, such as breast cancer, where the kit comprises a reaction body and one or more probes, such as LNA oligonucleotides as defined herein. The probes, such as LNA oligonucleotides are preferably immobilised onto said reactions body (e.g. by using the immobilising techniques described above).

For the kits according to the invention, the reaction body is preferably a solid support material, e.g. selected from borosilicate glass, soda-lime glass, polystyrene, polycarbonate, polypropylene, polyethylene, polyethyleneglycol terephthalate, polyvinylacetate, polyvinylpyrrolidinone, polymethylmethacrylate and polyvinylchloride, preferably polystyrene and polycarbonate. The reaction body may be in the form of a specimen tube, a vial, a slide, a sheet, a film, a bead, a pellet, a disc, a plate, a ring, a rod, a net, a filter, a tray, a microtitre plate, a stick, or a multi-bladed stick.

A written instruction sheet stating the optimal conditions for use of the kit typically accompanies the kits.

A preferred embodiment of the invention are kits for the characterisation of cancer, such as the cancers listed herein. Such kits may allow the detection or quantification of target non-coding RNAs, such as miRNAs, siRNAs, snRNAs, piRNAs, non-coding antisense transcripts or alternative splice variants.

The kit may comprise libraries of detection probes, which comprise one or more detection probes and optionally one or more control probes. The kit may also comprise detection probes for mRNAs (i.e. coding RNAs), and DNA, the presence or absence or level of which may also contribute to characterising the cancer. It is preferable that the kit comprises an array comprising a collection of detection probes, such as an oligonucleotide arrays or microarray.

The use of the kit therefore allows detection of non-coding RNAs which are associated with cancer, and whose level or presence or absence, may, either alone, or in conjunction with the level or presence or absence of other non-coding RNAs, and optionally coding RNAs, provide signal data which can be used to characterize said cancer.

In one aspect, the kit comprises in silico protocols for their use. The detection probes contained within these kits may have any or all of the characteristics described above. In one preferred aspect, a plurality of probes comprises at least one stabilizing nucleotide, such as an LNA nucleotide. In another aspect, the plurality of probes comprises a nucleotide coupled to or stably associated with at least one chemical moiety for increasing the stability of binding of the probe.

The invention therefore also provides for an array, such as a microarray which comprises one or more detection probe according to the invention, such as the collection of detection probes and optionally one or more control probe, preferably a collection of control probes. The array or microarray is particularly preferred for use in the method of the invention.

### Further Embodiments

It will be apparent that the following embodiments may apply to other forms of cancer other than breast cancer. In addition the following embodiments may be combined with further aspects of the invention as disclosed herein.

### Embodiments

1. A method for the characterisation of breast cancer, in a sample derived or obtained from a mammal, preferably a human being, said method comprising the following steps:
   a. Obtaining at least one test sample, such as a biopsy sample, of a tumor or of a putative tumor, from a patient, and optionally at least one control sample;
   b. Presenting a first population of nucleic acid molecules, prepared from said at least one test sample, and optionally a second population of nucleic acid molecules, prepared from said control sample;
   c. Hybridizing said first population of target molecules, and optionally said second population of target molecules, against at least one detection probe, wherein said at least one detection probe comprises a recognition sequence derived from a non-coding RNA sequence associated with said cancer, such as a non-coding RNA sequence selected from the group consisting of microRNA (miRNA), siRNA, piRNA, and snRNA, and precursor sequences thereof;
   d. Detecting a signal emitted during or subsequent to said hybridization step, said signal providing data which is indicative of hybridization of said at least one detection probe to a first complementary target within said first population of target molecules;
   e. Comparing said signal data obtained to reference data, which optionally maybe obtained from said control sample, to provide characterisation of at least one feature of said cancer.
2. The method according to embodiment 1, wherein said first and said second population of nucleic acid molecules comprises an RNA fraction which comprises non coding RNA, such as non coding RNA selected from the group consisting of microRNA (miRNA), siRNA piRNA, and snRNA, precursor non-coding RNA, such as pre-miRNA, pre-siRNA, and pre-piRNA.
3. The method according to embodiment 1, wherein said first and said second population of nucleic acid molecules comprises a population of target molecules derived from an RNA fraction which comprises non coding RNA, such as non coding RNA selected from the group consisting of microRNA (miRNA), siRNA piRNA, and snRNA precursor non-coding RNA, such as pre-miRNA, pre-siRNA, and pre-piRNA.
4. The method according to any of the preceding embodiments, wherein the at least one feature of said cancer is selected from one or more of the group consisting of: Presence or absence of said cancer; type of said cancer; origin of said cancer; diagnosis of cancer; prognosis of said cancer; therapy outcome prediction; therapy outcome monitoring; suitability of said cancer to treatment, such as suitability of said cancer to chemotherapy treatment and/or radiotherapy treatment; suitability of said cancer to hormone treatment; suitability of said cancer for removal by invasive surgery; suitability of said cancer to combined adjuvant therapy.
5. The method according to any one of the preceding embodiments, wherein the detection probe is an oligonucleotide or analogue thereof.
6. The method according to embodiment 5, wherein said oligonucleotide comprises at least one nucleotide analogue, such as a LNA.
7. The method according to embodiment 5 or 6, wherein said oligonucleotide comprises less than 21 nucleotide or nucleotide analogue units, such as less than 18 nucleotide or nucleotide analogue units.
8. The method according to embodiment 7, wherein the oligonucleotide comprises between 8 and 16, such as between 12 and 14 nucleotide or nucleotide analogue units.
9. The method according to any one of embodiments 5 to 8, wherein the oligonucleotide comprises nucleotide analogues inserted with regular spacing between said nucleoside analogues, e.g. at every second nucleotide position, every third nucleotide position, or every fourth nucleotide position.
10. The method according to any one of the preceding embodiments, wherein the detection probe or probes, are derived from, or are capable of selectively hybridizing to, one or more mammalian non-coding RNAs such as those selected from the group of mature miRNAs, mature siRNAs, mature piRNAs and mature snRNAs.
11. The method according to any one of the preceding embodiments, wherein the detection probe or probes, are derived from, or are capable of selectively hybridizing to, one or more mammalian non-coding RNAs selected from the group consisting of of pre-miRNAs, pre-siRNAs, pre-piRNAs and pre-snRNAs.
12. The method according to any one of the preceding embodiments, wherein the detection probe or probes, are derived from or are capable of selectively hybridizing to, one or more mammalian miRNAs or siRNA.
13. The method according to any one of the preceding embodiments, wherein the detection probe or probes, are derived from, or are capable of selectively hybridizing to, one or more mammalian piRNAs.
14. The method according to any one of the preceding embodiments, wherein the detection probe or probes, are derived from, or are capable of selectively hybridizing to, one or more mammalian snRNAs, such a human U6RNA, such as SEQ ID No 113.
15. The method according to any one of the preceding embodiments, wherein the one or more mammalian non-coding RNAs are naturally found in one or more of the group consisting of humans, mice and rats, preferably humans.
16. The method according to any one of the preceding embodiments, wherein the one or more non-coding RNAs are selected from the group consisting of: SEQ ID No. 4; SEQ ID No. 72; SEQ ID No. 36; SEQ ID No. 29; SEQ ID No. 44; SEQ ID No. 65; SEQ ID No. 76; SEQ ID No. 12; SEQ ID No. 28; SEQ ID No. 83; SEQ ID No. 52; SEQ ID No. 75; SEQ ID No. 91; SEQ ID No. 9; SEQ ID No. 85; SEQ ID No. 92; SEQ ID No. 26; SEQ ID No. 14; SEQ ID No. 46; SEQ ID No. 39; SEQ ID No. 69; SEQ ID No. 66; SEQ ID No. 6; SEQ ID No. 64; SEQ ID No. 84; SEQ ID No. 93; SEQ ID No. 54; SEQ ID No. 24; SEQ ID No. 42; SEQ ID No. 94; SEQ ID No. 95; SEQ ID No. 18; SEQ ID No. 90; SEQ ID No. 87; SEQ ID No. 6; SEQ ID No. 82; SEQ ID No. 23; SEQ ID No. 55; SEQ ID No. 57; SEQ ID No. 33; SEQ ID No. 88; SEQ ID No. 37; SEQ ID No. 96; SEQ ID No. 97; SEQ ID No. 85; SEQ ID No. 55; SEQ ID No. 53; SEQ ID No. 58; SEQ ID No. 68; SEQ ID No. 59; SEQ ID No. 73; SEQ ID No. 41; SEQ ID No. 19; SEQ ID No. 67; SEQ ID No. 89; SEQ ID No. 76; SEQ ID No. 45; SEQ ID No. 63; SEQ ID No. 25; SEQ ID No. 62; SEQ ID No. 21; SEQ ID No. 78; SEQ ID No. 13; SEQ ID No. 50; SEQ ID No. 3; SEQ ID No. 27; SEQ ID No. 10; SEQ ID No. 38; SEQ ID No. 47 ; SEQ ID No. 77; SEQ ID No. 51; SEQ ID No. 11; SEQ ID No. 30; SEQ ID No. 43; SEQ ID No. 22; SEQ ID No. 1; SEQ ID No. 40; SEQ ID No. 48; SEQ ID No 111; SEQ ID No 112; SEQ ID No 113; and SEQ ID No. 32; SEQ ID No 219; SEQ ID No 220; SEQ ID No 221; SEQ ID No 222; SEQ ID No 223; SEQ ID No 224; SEQ ID No 225; SEQ ID No 226; SEQ ID No 227; SEQ ID No 349; SEQ ID No 350; SEQ ID No 351; SEQ ID No 352; SEQ ID No 353; SEQ ID No 354; SEQ ID No 355; SEQ ID No 356; SEQ ID No 357; SEQ ID No 358; SEQ ID No 359; SEQ ID No 360; SEQ ID No 361; SEQ ID No 362; SEQ ID No 363; SEQ ID No 364; SEQ ID No 365; SEQ ID No 366; SEQ ID No 367; and allelic variants thereof.
17. The method according to embodiment 16, wherein the one or more non-coding RNAs are selected from the group consisting of: SEQ ID 45; SEQ ID 13; SEQ ID 113; SEQ ID No 219; SEQ ID No 220; SEQ ID No 221; SEQ ID No 222; SEQ ID No 223; SEQ ID No 224; SEQ ID No 225; SEQ ID No 226; SEQ ID No 227; and allelic variants thereof.
18. The method according to embodiment 17, wherein the one or more non-coding RNAs are selected from the group consisting of: SEQ ID 113 and SEQ ID No 227; and allelic variants thereof.
19. The method according to any one of embodiments 16 to 18, wherein said one or more non-coding RNAs are the said first complementary target.
20. The method according to any one of the preceding embodiments, wherein the detection probe or probes are capable of selectively hybridizing to the precursor form of the non-coding RNA, but are not capable of selectively hybridizing to the mature form of the non-coding RNA.
21. The method according to any one of embodiments 1 to 15, wherein the one or more non-coding RNAs are selected from the group consisting of: SEQ ID No 237; SEQ ID No 238; SEQ ID No 239; SEQ ID No 240; SEQ ID No 241; SEQ ID No 242; SEQ ID No 243; SEQ ID No 244; SEQ ID No 245; SEQ ID No 246; SEQ ID No 247; SEQ ID No 248; SEQ ID No 249; SEQ ID No 250; SEQ ID No 251; SEQ ID No 252; SEQ ID No 253; SEQ ID No 254; SEQ ID No 255; SEQ ID No 256; SEQ ID No 257; SEQ ID No 258; SEQ ID No 259; SEQ ID No 260; SEQ ID No 261; SEQ ID No 262; SEQ ID No 263; SEQ ID No 264; SEQ ID No 265; SEQ ID No 266; SEQ ID No 267; SEQ ID No 268; SEQ ID No 269; SEQ ID No 270; SEQ ID No 271; SEQ ID No 272; SEQ ID No 273; SEQ ID No 274; SEQ ID No 275; SEQ ID No 276; SEQ ID No 277; SEQ ID No 278; SEQ ID No 279; SEQ ID No 280; SEQ ID No 281; SEQ ID No 282; SEQ ID No 283; SEQ ID No 284; SEQ ID No 285; SEQ ID No 286; SEQ ID No 287; SEQ ID No 288; SEQ ID No 289; SEQ ID No 290; SEQ ID No 291; SEQ ID No 292; SEQ ID No 293; SEQ ID No 294; SEQ ID No 295; SEQ ID No 296; SEQ ID No 297; SEQ ID No 298; SEQ ID No 299; SEQ ID No 300; SEQ ID No 301; SEQ ID No 302; SEQ ID No 303; SEQ ID No 304; SEQ ID No 305; SEQ ID No 306; SEQ ID No 307; SEQ ID No 308; SEQ ID No 309; SEQ ID No 310; SEQ ID No 311; SEQ ID No 312; SEQ ID No 313; SEQ ID No 314; SEQ ID No 315; SEQ ID No 316; SEQ ID No 317; SEQ ID No 318; SEQ ID No 319; SEQ ID No 320; SEQ ID No 321; SEQ ID No 322; SEQ ID No 323; SEQ ID No 324; SEQ ID No 325; SEQ ID No 326; SEQ ID No 327; SEQ ID No 328; SEQ ID No 329; SEQ ID No 330; SEQ ID No 331; SEQ ID No 332; SEQ ID No 333; SEQ ID No 334; SEQ ID No 335; SEQ ID No 336; SEQ ID No 337; SEQ ID No 338; SEQ ID No 339; SEQ ID No 340; SEQ ID No 341; SEQ ID No 342; SEQ ID No 343; SEQ ID No 344; SEQ ID No 345; SEQ ID No 346; SEQ ID No 347; SEQ ID No 348; and allelic variants thereof.
22. The method according to embodiment 21, wherein the one or more non-coding RNAs are selected from the group consisting of: SEQ ID No 340; SEQ ID No 341; SEQ ID No 342; SEQ ID No 343; SEQ ID No 344; SEQ ID No 345; SEQ ID No 346; SEQ ID No 347; SEQ ID No 348; and allelic variants thereof.
23. The method according to embodiment 21 or 22, wherein the RNA is a mature non-coding RNA, such as a mature miRNA.
24. The method according to any one of embodiments 21 to 23, wherein said one or more non-coding RNAs are the said first complementary target.
25. The method according to embodiment any one of the preceding embodiments, wherein the one or more detection probe oligonucleotides are selected from the group comprising: SEQ ID No. 114, SEQ ID No. 115, SEQ ID No. 116, SEQ ID No. 117, SEQ ID No. 118, SEQ ID No. 119, SEQ ID No. 120, SEQ ID No. 121, SEQ ID No. 122, SEQ ID No. 123, SEQ ID No. 124, SEQ ID No. 125, SEQ ID No. 126, SEQ ID No. 127, SEQ ID No. 128, SEQ ID No. 129, SEQ ID No. 130, SEQ ID No. 131, SEQ ID No. 132, SEQ ID No. 133, SEQ ID No. 134, SEQ ID No. 135, SEQ ID No. 136, SEQ ID No. 137, SEQ ID No. 138, SEQ ID No. 139, SEQ ID No. 140, SEQ ID No. 141, SEQ ID No. 142, SEQ ID No. 143, SEQ ID No. 144, SEQ ID No. 145, SEQ ID No. 147, SEQ ID No. 148, SEQ ID No. 149, SEQ ID No. 150, SEQ ID No. 151, SEQ ID No. 152, SEQ ID No. 153, SEQ ID No. 154, SEQ ID No. 155, SEQ ID No. 156, SEQ ID No. 157, SEQ ID No. 158, SEQ ID No. 159, SEQ ID No. 160, SEQ ID No. 161, SEQ ID No. 162, SEQ ID No. 163, SEQ ID No. 164, SEQ ID No. 165, SEQ ID No. 166, SEQ ID No. 167, SEQ ID No. 168, SEQ ID No. 169, SEQ ID No. 170, SEQ ID No. 171, SEQ ID No. 172, SEQ ID No. 173, SEQ ID No. 174, SEQ ID No. 175, SEQ ID No. 176, SEQ ID No. 177, SEQ ID No. 178, SEQ ID No. 179, SEQ ID No. 180, SEQ ID No. 181, SEQ ID No. 182, SEQ ID No. 183, SEQ ID No. 184, SEQ ID No. 185, SEQ ID No. 186, SEQ ID No. 187, SEQ ID No. 188, SEQ ID No. 189, SEQ ID No. 190, SEQ ID No. 191, SEQ ID No. 192, SEQ ID No. 193, SEQ ID No. 194, SEQ ID No. 195, SEQ ID No. 196, SEQ ID No. 197, SEQ ID No. 198, SEQ ID No. 199, SEQ ID No. 200, SEQ ID No. 201, SEQ ID No. 202, SEQ ID No. 203, SEQ ID No. 204, SEQ ID No. 205, SEQ ID No. 206, SEQ ID No. 207, SEQ ID No. 208, SEQ ID No. 209, SEQ ID No. 210, SEQ ID No. 211, SEQ ID No. 212, SEQ ID No. 213, SEQ ID No. 214, SEQ ID No. 215, SEQ ID No. 216, SEQ ID No. 217, SEQ ID No. 218; SEQ ID No 228; SEQ ID No 229; SEQ ID No 230; SEQ ID No 231; SEQ ID No 232; SEQ ID No 233; SEQ ID No 234; SEQ ID No 235; SEQ ID No 236; and variants, homologues and fragments thereof
26. The method according to embodiment 25, wherein the one or more detection probe oligonucleotides are selected from the group comprising: SEQ ID 175; SEQ ID 181; SEQ ID 120; SEQ ID 121; SEQ ID No 228; SEQ ID No 229; SEQ ID No 230; SEQ ID No 231; SEQ ID No 232; SEQ ID No 233; SEQ ID No 234; SEQ ID No 235; SEQ ID No 236; and variants, homologues and fragments thereof
27. The method according to embodiment 25 or 26, wherein the one or more detection probe oligonucleotides are selected from the group comprising: tCcaTaaAgtAggAaaCacTaca; CtcAgtAatGgtAacGgt; AaaCtcAgtAatGgtAacGg; tccAtcAtcAaaAcaAatGgaGt; gaAcaGgtAgtCtgAacActGgg; tCtgTatCgtTccAatTt; GcgTgtCatCctTgcg; gaAtcTtgTccCgcAggt; gAacAggTagTctAaaCacTg; ggActTtgAggGccAgtt; aacCaaTgtGcaGacTacTgta; gGgcCtcCacTttGat; aTaaGgaTttTtaGggGcaTt; cAcaAacCatTatGtgCtgCta; gGcgAccCagAgg; acaGttCttCaaCtgGcaGctt; ctAccAtaGggTaaAacCact; aGtgCttCccTccAgag; aaCaaCcaGctAagAcaCtgCca; tgtAaaCcaTgaTgtGctGcta; ccAggTtcCacCccAgcAggc; ctGccTgtCtgTgcCtgCtgt; AaaGtgCatCccTctGga; acaCccCaaAatCgaAgcActTc; acaAagTtcTgtGatGcaCtga; gAacTgcCttTctCtcCa; agTgcTtcTtaCctCcaGa; AagTgcCccCatAgtTtgA; AacTgtTccCgcTgcTa; gcGgaActTagCcaCtgTgaa; GggGtaTttGacAaaCtgAca; gaGacCcaGtaGccAgaTgtAgct; cTtcCagTcgAggAtgTttAca; caAaaGagCccCcaGtt; tcCagTcaAggAtgTttAca; acTagActGtgAgcTccTc; ctCaaAggGctCctCag; acaAagTtcTgtGatGcaCtga; gGagAgcCagGagAa; gacGggTgcGatTtcTgtGtgAga; gCcaAtaTttCtgTgcTgcTa; gcAgaActTagCcaCtgTgaa; ctgGagGaaGggCccAgaGg; AccGacCgaCcgAtc; aGccTatGgaAttCagTtcTca; gGccCtgTgcTttGc; gGagCctCagTctAgt; tCcgTggTtcTacCctg; gCcaAtaTttCtgTgcTgcTa; aCtgTacAaaCtaCtaCctCa; gAaaCccAgcAgaCaaTgtAgct; aaGacGggAggAgag; gCtgAgaGtgTagGatGttTaca; aCcgAttTcaAatGgtGcta; acAggAttGagGggGggCcct; actAtaCaaCctCAccTca; aaCtaTacAatCtaCtaCctCa; AagAacAgcCctCctCtg; gAacAgaTagTctAaaCacTggg; tCaaCatCagTctGatAagCta; ttTtcCcaTgcCctAtaCct; gcAagCccAgaCcgCaaAaag; aaTgaCacCtcCctGtga; aGagGttTccCgtGtaTg; gcAttAttActCacGgtAcga; aCagCacAaaCtaCtaCctCa; gGaaAtcCctGgcAatGtgAt; gAaaAacGccCccTgg; cTgtTccTgcTgaActGagCca; ccaAtaTttAcgTgcTgcTa; tTcgCccTctCaaCccAgcTttt; caGacTccGgtGgaAtgAagGa; ccAtcAttAccCggCagTatTa; cAtcAttAccAggCagTatTaga; cacAagTtcGgaTctAcgGgtt; aaCcaTacAacCtaCtaCctCa; aaCcaCacAacCtaCtaCctCa; cCatCttTacCagAcaGtgTta; atcCaaTcaGttCctGatGcaGta; aaCtaTacAacCtaCtaCctCa; tcaCaaGttAggGtcTcaGgga; taGctGgtTgaAggGgaCcaa; GggActTtgTagGccAg; cTtcAgtTatCacAgtActg; tCctGggAaaActGga; cAtaCagCtaGatAacCaaAga; caCcaTtgTcaCacTccA; GaaAgaGacCggTtcActG; AgtGaaGacAcgGagC; acAggTtaAagGgtCtcAg; AgcTacAgtGctTcaTctCa; cCatCatCaaAacAaaTggAg; and variants, homologues and fragments therof.
28. The method according to embodiment 27, wherein the one or more detection probe oligonucleotides are selected from the group comprising: tCaaCatCagTctGatAagCta; aCagCacAaaCtaCtaCctCa; GcgTgtCatCctTgcg; gaAtcTtgTccCgcAggt; cTtcAgtTatCacAgtActg; tCctGggAaaActGga; cAtaCagCtaGatAacCaaAga; caCcaTtgTcaCacTccA; GaaAgaGacCggTtcActG; AgtGaaGacAcgGagC; acAggTtaAagGgtCtcAg; AgcTacAgtGctTcaTctCa; cCatCatCaaAacAaaTggAg; and variants, homologues and fragments therof.
29. The method according to any one of the preceding embodiments, wherein the RNA fraction presented from the said test sample and optionally said control sample are obtained by extracting RNA from said test and/or control sample.
30. The method according to embodiment 29, wherein the RNA fraction is in the form of a nucleic acid fraction comprising both DNA and RNA, a total RNA fraction or a small RNA enriched fraction, such as an miRNA enriched fraction.
31. The method according to any one of the preceding embodiments, wherein at least one control sample is obtained, and the second population of nucleic acids from the at least one control sample is also presented and hybridized against at least one detection probe, wherein said characterisation is obtained in step e) by comparing the signal obtained by the control sample to the signal obtained from the test sample.
32. The method according to any one of the preceding embodiments, wherein the at least one control sample is obtained from the same patient.
33. The method according to any one of the preceding embodiments, wherein the at least one control sample is obtained from a non tumorous tissue.
34. The method according to any one of the preceding embodiments, wherein the control sample is obtained from tissue adjacent to said putative tumor, and/or from an equivalent position elsewhere in the body.
35. The method according to any one of embodiments 1 to 32, wherein the at least one control sample is obtained from a tumor tissue.
36. The method according to anyone of the preceding embodiments, wherein the hybridization signal obtained from the test sample is higher than the hybridization signal obtained from the control sample.
37. The method according to anyone of embodiments 1-35, wherein the hybridization signal obtained from the test sample is lower than the hybridization signal obtained from the control sample.
38. The method according to any one of the previous embodiments where at least two control samples are obtained, one control sample being obtained from said patient according to any of the preceding embodiments, and at least one further control sample being obtained from a previously obtained sample of a cancer, which may originate from the same patient or a different patient.
39. The method according to embodiment 38, wherein the hybridization signal obtained from the at least one further test sample is equivalent to or greater than the signal obtained from the either the signal obtained from the first control sample and/or the signal obtained from the test sample.
40. The method according to embodiment 38, wherein the hybridization signal obtained from the at least one further test sample is less than the signal obtained from the either the signal obtained from the first control sample and/or the signal obtained from the test sample.
41. The method according to any of the preceding embodiments, wherein the test and control samples are hybridized to said at least one detection probe simultaneously, either in parallel hybridizations or in the same hybridization experiment.
42. The method according to any one of the preceding embodiments, wherein the test and control sample or samples are hybridized to said at least one detection probe sequentially, either in the same hybridization experiment, or different hybridization experiments.
43. The method according to any of the previous embodiments wherein an additional step is performed prior to step c), said step comprising performing quantitative analysis of the RNA population obtained from said test sample, and optionally from said control sample or samples.
44. The method according to embodiment 43, wherein the hybridization step in step c) occurs in silico, for example by virtual hybridization.
45. The method according to embodiment 43 or 44, wherein the hybridization step is performed by via quantative analysis of the target non-coding RNAs present in said test sample and comparison to equivalent quantitative analysis performed on said one or more control samples.
46. The method according to any of one of the preceding embodiments, wherein the non coding RNA is a microRNA or siRNA.
47. The method according to any one of embodiments 1 to 45, wherein the non coding RNA is a piRNA.
48. The method according to any one of embodiments 1 to 45, wherein the non coding RNA is a small nucleolar RNA (snRNA).
49. The method according to any of the preceding embodiments, wherein at least one further detection probe is used, wherein said at least one further detection probe is derived from or is capable of selectively hybridizing with a further complementary target, selected from the group consisting of: a pre-miRNA molecule; a pre-siRNA molecule; and a pre-piRNA molecule.
50. The method according to embodiment 49, wherein said further complementary target is a precursor form of said first complementary target, or complementary target derived from said precursor form of said first complementary target, wherein said first complementary target is in the form of a mature non-coding RNA.
51. The method according to embodiment 50 wherein the at least one further detection probe is capable of hybridizing to the loop region of said further complementary target, such as a precursor miRNA, a precursor siRNA or a precursor piRNA, such as precursor non-coding RNAs according to embodiments 15 to 17, or an equivalent position in a further complementary target derived therefrom.
52. The method according to any of one the preceding embodiments, wherein the hybridization step is performed against at least one detection probe pair, said detection probe pair comprising of a first detection probe which is capable of hybridizing to said further complementary target, such as a precursor non-coding RNA, such as those according to embodiments 16 to 18, and a second detection probe which is capable of hybridizing to said first complementary target, such as the corresponding mature non-coding RNA, such as those according to embodiments 21 and 22.
53. The method according to any of one the preceding embodiments, wherein the hybridization step is performed against a collection of said detection probes, said collection of detection probes comprising at least 5 detection probes, such as at least 10 detection probes.
54. The method according to embodiment 53, wherein the hybridization step is performed against a collection of detection probes comprising least 30 detection probes, such as at least 50 detection probes.
55. The method according to embodiment 53 or 54, wherein said collection of detection probes comprises at least one detection probe pair according to embodiment 52.
56. The method according to embodiment 55, wherein said collection of detection probes comprises at least two non identical detection probe pairs, such as at least 5 non identical detection probe pairs, such as at least 10 non identical detection probe pairs, such as at least 20 non identical detection probe pairs.
57. The method according to any one of embodiments 52 to 56, wherein the hybridization step is performed against an oligonucleotide array, such as a micro array, wherein said oligonucleotide array comprises said at least one detection probe, and/or at least one detection probe pairs.
58. The method according to any one of embodiments 1 to 52, wherein the hybridization occurs in situ, in or on the biopsy samples
59. The method according to embodiment 58, wherein said in situ hybridization consists of the simultaneous or sequential hybridization of between 1 and 10 detection probes, such as between 3 and 10 detection probes, such as no more than three detection probes.
60. The method according to embodiment 59, wherein said in situ hybridization consists of simultaneous hybridization of a detection probe pair, such as the detection probe pair according to embodiment 52.
61. The method according to any one of embodiments 1 to 57, wherein the detection probe or each member of said collection of collection of detection probes are linked to a bead, and wherein said detection of hybridization occurs via bead based detection.
62. The method according to any one of the previous embodiments, wherein the hybridization step comprises a polymerase chain reaction (PCR).
63. The method according to embodiment 62, wherein said PCR comprises q-PCR and/or real time PCR (RT-PCR).
64. The method according to any one of embodiments 1 to 57, wherein the hybridization steps comprises northern blotting.
65. The method according to any one of embodiments 1 to 57, wherein the hybridization steps comprises an RNase protection assay (RPA).
66. Use of at least one detection probe as defined in any preceding embodiment, such as a microRNA (miRNA), siRNA or snRNA, or precursor thereof, for the characterisation of breast cancer, wherein said detection probe hybridizes to at least one non coding mRNA, or precursor thereof associated with breast cancer,
67. A collection of detection probes, wherein each member of said collection comprises a recognition sequence consisting of nucleobases and/or affinity enhancing nucleobase analogues, wherein said collection of detection probes comprises at least one member which is selected for its ability to hybridize to one or more non-encoding RNAs which are associated with breast cancer, wherein said one or more non-encoding RNAs are as defined in any preceding embodiment.
68. A collection of detection probes according to embodiment 67, wherein said collection of detection probes comprises at least one detection probe pair according to embodiment 52.
69. A kit for the detection of breast cancer, said kit comprising at least one detection probe or detection probe pair according to any previous embodiment.
70. A kit for the detection of breast cancer according to embodiment 69, wherein said kit comprises a collection of detection probes according to embodiments 52 to 57 or 67.
71. A kit for the detection of breast cancer according to embodiment 69 or 70, wherein said kit is in the form or comprises an oligonucleotide array, according to embodiment 57.
72. A method of for the treatment of breast cancer, said method comprising
   a. Isolating at least one tissue sample from a patient suffering from breast cancer;
   b. Performing the characterisation of the at least one tissue sample according to embodiments any one of embodiments 1 to 65 and/or utilising the collection of detection probes according to embodiments 67 or 68 or the kit according to any one of embodiments 69 to 71, to identify at least one feature of said cancer;
   c. Based on the at least one feature identified in step b) diagnosing the physiological status of the cancer disease in said patient;
   d. Selecting an appropriate form of therapy for said patient based on the said diagnosis;
   e. Administering said appropriate form of therapy.
73. The method of for the treatment of breast cancer according to embodiment 72, wherein the at least one feature of said cancer is selected from one or more of the group consisting of: Presence or absence of said cancer; type of said cancer; origin of said cancer; diagnosis of cancer; prognosis of said cancer; therapy outcome prediction; therapy outcome monitoring; suitability of said cancer to treatment, such as suitability of said cancer to chemotherapy treatment and/or radiotherapy treatment; suitability of said cancer to hormone treatment; suitability of said cancer for removal by invasive surgery; suitability of said cancer to combined adjuvant therapy.
74. The method of for the treatment of breast cancer according to embodiment 73, wherein the at least one feature of said cancer is determination of the origin of said cancer, wherein said cancer is a metestasis and/or a secondary cancer which is remote from the cancer of origin, such as the primary cancer.
75. The method for the treatment of breast cancer according to embodiment 73 or 74, wherein the treatment comprises one or more of the therapies selected from the group consisting of: chemotherapy; hormone treatment; invasive surgery; radiotherapy; and adjuvant systemic therapy.
76. A method for the determination of suitability of a cancer patient for treatment comprising:
   a. Isolating at least one tissue sample from a patient suffering from breast cancer;
   b. Performing the characterisation of the at least one tissue sample according to embodiments any one of embodiments 1 to 65 and/or utilising the collection of detection probes according to embodiments 67 or 68 or the kit according to any one of embodiments 69 to 71, to identify at least one feature of said cancer;
   c. Based on the at least one feature identified in step b) diagnosing the physiological status of the patient;
   d. Based on the said diagnosis obtained in step c) determining whether said patient would benefit from treatment of said breast cancer.
77. The method of for the determination of suitability of a cancer for treatment according to embodiment 76, wherein the at least one feature of said cancer is selected from one or more of the group consisting of: Presence or absence of said cancer; type of said cancer; origin of said cancer; diagnosis of cancer; prognosis of said cancer; therapy outcome prediction; therapy outcome monitoring; suitability of said cancer to treatment, such as suitability of said cancer to chemotherapy treatment and/or radiotherapy treatment; suitability of said cancer to hormone treatment; suitability of said cancer for removal by invasive surgery; suitability of said cancer to combined adjuvant therapy.
78. The method of for the treatment of breast cancer according to embodiment 77, wherein the at least one feature of said cancer is determination of the origin of said cancer, wherein said cancer is a metastasis and/or a secondary cancer which is remote from the cancer of origin, such as the primary cancer.
79. A method according for the determination of the origin of a metastatic (such as secondary) cancer, or a cancer suspected of being a metastasis, comprising:
   a. Isolating at least one tissue sample from a patient suffering from cancer, such as breast cancer, or a cancer which may have originated from a breast cancer tumor;
   b. Performing the characterisation of the at least one tissue sample according to embodiments any one of embodiments 1 to 65 and/or utilising the collection of detection probes according to embodiments 67 or 68 or the kit according to any one of embodiments 69 to 71, to identify the origin of said metastatic cancer.
80. A method for the determination of the origin of a metastatic cancer, or a cancer suspected of being a metastasis, according to embodiment 79, wherein said characterisation comprises comparison of the at least on feature with the equivalent at least one feature obtained from at least one control sample, wherein said control sample is derived from a cancer of known physiological origin.
81. A method for the determination of the likely prognosis of a breast cancer patient comprising:
   a. Isolating at least one tissue sample from a patient suffering from breast cancer;
   b. Performing the characterisation of the at least one tissue sample according to embodiments any one of embodiments 1 to 65 and/or utilising the collection of detection probes according to embodiments 67 or 68 or the kit according to any one of embodiments 69 to 71, to identify at least one feature of said cancer;
   c. wherein said feature allows for the determination of the likely prognosis of said cancer patient.
82. A method for specific isolation, purification, amplification, detection, identification, quantification, inhibition or capture of a target nucleotide sequence in a sample, said method comprising contacting said sample with a detection probe as defined in any one of embodiments 1 to 65 under conditions that facilitate hybridization between said member/probe and said target nucleotide sequence, wherein said target nucleotide sequence is, or is derived from a non-coding RNA associated with breast cancer.

### EXAMPLES

The invention will now be further illustrated with reference to the following examples. It will be appreciated that what follows is by way of example only and that modifications to detail may be made while still falling within the scope of the invention.

LNA-substituted probes may be prepared according to Example 1 of PCT/DK2005/000838.

### Example 1

Molecular Classification of Breast Cancer by MicroRNA Signatures

breast cancer is the most frequent form of cancer among women worldwide. Currently, treatment and prognosis is based on clinical and histo-pathological graduation, such as TNM classification (tumor size, lymph node and distant metastases status) and estrogen receptor status. To improve both the selection of therapy and the evaluation of treatment response, more accurate determinants for prognosis and response, such as molecular tumor markers, are needed. The primary aim of this study was to study the expression patterns of microRNAs (miRNAs) in tumors and normal breast tissue to identify new molecular markers of breast cancer.

Biopsies from primary tumors and from the proximal tissue (1 cm from the border zone of tumor) were collected from female patients (age 55-69) undergoing surgery for invasive ductal carcinoma. Total-RNA was extracted following the “Fast RNA GREEN” protocol from Bio101. Assessment of miRNA levels was carried out on miRCURY™ microarrays according to the manufacturers recommended protocol (Exiqon, Denmark).

The results from the miRNA analysis revealed numerous differentially expressed miRNAs, including those reported earlier to be associated with breast cancer, such as let-7a/d/f, miR-125a/b, miR-21, miR-32, and miR-136 [1]. In addition, we have identified several miRNAs that have not previously been connected with breast cancer.

RNA Extraction

Before use, all samples were kept at −80° C.

Two samples—ca. 100 mg of each—were used for RNA extraction:

PT (primary tumor)

1C (normal adjacent tissue, one cm from the primary tumor)

The samples were thawed on ice, and kept in RNAlater® (Cat #7020, Ambion) during disruption with a sterile scalpel into smaller ca. 1 mm wide slices.

To a FastPrep GREEN (Cat #6040-600, Bio101) tube containing lysis matrix was added:

500 μL CRSR-GREEN

500 μL PAR

100 μL CIA

200 μL tissue

The tubes were placed in the FastPrep FP120 cell disruptor (Bio101) and run for 40 seconds at speed 6. This procedure was repeated twice, before cooling on ice for 5 min. The tubes were centrifuged at 4° C. and at maximum speed in an Eppendorf microcentrifuge for 10 min to enable separation into organic and water phases. The upper phase from each vial was transferred to new Eppendorf 1.5 mL tubes while avoiding the interphase. 500 μl CIA was added, vortexed for 10 seconds, and spun at max speed for 2 min to separate the phases. Again, the top phase was transferred to new Eppendorf tubes, while the interphase was untouched. 500 μL DIPS was added, vortexed, and incubated at room temperature for 2 min. The tubes were centrifuged for 5 min at max speed to pellet the RNA. The pellet was washed twice with 250 μL SEWS and left at room temperature for 10 min to air dry. 50 μL SAFE was added to dissolve the pellet, which was stored at −80° C. until use. QC of the RNA was performed with the Agilent 2100 BioAnalyser using the Agilent RNA6000Nano kit. RNA concentrations were measured in a NanoDrop ND-1000 spectrophotometer. The PT was only 71 ng/μL, so it was concentrated in a speedvac for 15 min to 342 ng/μL. The 1C was 230 ng/μL, and was used as is.

RNA Labelling and Hybridization

Essentially, the instructions detailed in the “miRCURY Array labelling kit Instruction Manual” were followed:

All kit reagents were thawed on ice for 15 min, vortexed and spun down for 10 min.

In a 0.6 mL Eppendorf tune, the following reagents were added:

2.5× labelling buffer, 8 μL

Fluorescent label, 2 μL

1 μg total-RNA (2.92 μL (PT) and 4.35 μL (1C))

Labeling enzyme, 2 μL

Nuclease-free water to 20 μL (5.08 μL (PT) and 3.65 μL (1C))

Each microcentrifuge tube was vortexed and spun for 10 min.

Incubation at 0° C. for 1 hour was followed by 15 min at 65° C., then the samples were kept on ice.

For hybridization, the 12-chamber TECAN HS4800Pro hybridization station was used.

25 μL 2× hybridization buffer was added to each sample, vortexed and spun.

Incubation at 95° C., for 3 min was followed by centrifugation for 2 min.

The hybridization chambers were primed with 1× Hyb buffer.

50 μl of the target preparation was injected into the Hyb station and incubated at 60° C. for 16 hours (overnight).

The slides were washed at 60° C., for 1 min with Buffer A twice, at 23° C. for 1 min with Buffer B twice, at 23° C. for 1 min with Buffer C twice, at 23° C. for 30 sec with Buffer C once.

The slides were dried for 5 min.

Scanning was performed in a ScanArray 4000XL (Packard Bioscience).

Results

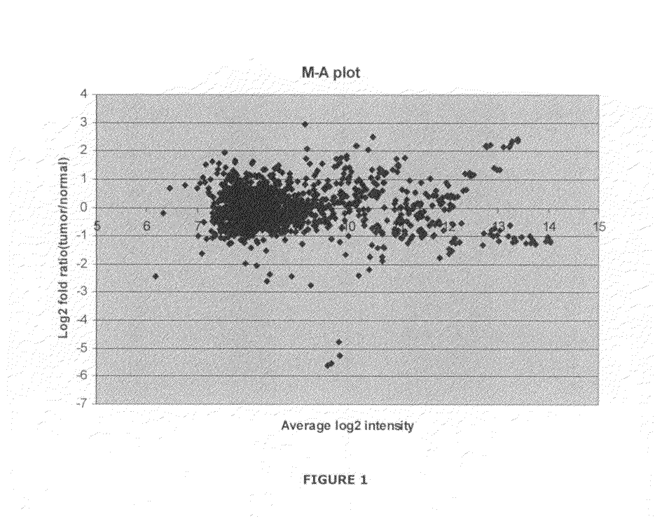

The M-A plot (FIG. 1) shows the Log2 fold ratio of tumor/normal (M) as a function of the Log2

TABLE 1
New, upregulated miRNAs in breast cancer compared to normal
breast tissue.

| | | | raw m | raw m | A | |
|---|---|---|---|---|---|---|
| GeneID | miRNA | fold | Tumor | Normal | mean | 2\hat{ }Am |
| 10947 | miR-142-3p | 4.7 | 3285.5 | 499.6 | 10.3 | 1271.7 |
| 11248 | miR-451 (11248) | 4.5 | 19211.4 | 2583.8 | 12.8 | 7043.1 |
| 11115 | miR-451 (11115) | 4.4 | 24572.8 | 3349.2 | 13.1 | 9064.7 |
| 10943 | miR-136 | 3.2 | 1843.6 | 393.9 | 9.7 | 827.9 |
| 10986 | miR-193a | 3.0 | 1888.4 | 472.6 | 9.9 | 942.1 |
| 10994 | miR-199a | 2.9 | 4104.3 | 980.8 | 11.0 | 2003.1 |
| 11278 | U6-snRNA-1 | 2.8 | 4016.5 | 970.6 | 10.9 | 1952.9 |
| 11279 | U6-snRNA-2 | 2.5 | 15114.0 | 3610.0 | 12.8 | 7290.0 |
| 11124 | miR-492 | 2.5 | 2061.8 | 625.0 | 10.1 | 1092.3 |
| 11205 | No known Hs target | 2.3 | 3126.3 | 938.2 | 10.7 | 1712.5 |
| 10987 | miR-193b | 2.3 | 807.6 | 377.7 | 9.1 | 538.2 |
| 10995 | miR-199a* | 2.3 | 10612.3 | 2899.9 | 12.4 | 5542.3 |
| 11214 | No known Hs target | 2.2 | 965.9 | 428.8 | 9.3 | 634.6 |
| 11078 | miR-365 | 2.2 | 1760.1 | 594.0 | 10.0 | 1020.7 |
| 10965 | miR-15a | 2.2 | 1317.7 | 500.0 | 9.7 | 804.1 |
| 11270 | No known Hs target | 2.2 | 2070.5 | 680.8 | 10.2 | 1174.5 |
| 11020 | miR-22 | 2.1 | 1859.4 | 639.5 | 10.1 | 1084.1 |
| 4700 | miR-140 | 2.1 | 452.4 | 322.6 | 8.6 | 378.5 |
| 13131 | miR-518c* | 2.0 | 250.1 | 254.3 | 8.0 | 251.4 |
| 11072 | miR-34a | 1.9 | 474.6 | 346.7 | 8.6 | 401.0 |
| 10966 | miR-15b | 1.9 | 2445.8 | 893.6 | 10.5 | 1476.5 |
| 11082 | miR-370 | 1.9 | 1018.1 | 483.5 | 9.4 | 698.3 |
| 11014 | miR-214 | 1.9 | 5347.9 | 1820.2 | 11.6 | 3060.2 |
| 11175 | miR-525 | 1.9 | 362.0 | 326.9 | 8.4 | 338.3 |
| 11086 | miR-373* | 1.8 | 4260.4 | 1629.3 | 11.2 | 2338.4 |
| 10956 | miR-148b | 1.8 | 422.5 | 345.0 | 8.6 | 377.9 |
| 5560 | miR-185 | 1.6 | 1781.1 | 804.0 | 10.2 | 1194.9 |
| 11151 | miR-516-5p | 1.6 | 222.7 | 282.0 | 7.9 | 245.4 |
| 11212 | No known Hs target | 1.6 | 4914.2 | 1948.4 | 11.6 | 3014.9 |
| 11135 | miR-503 | 1.5 | 2099.1 | 988.2 | 10.5 | 1438.0 |
| 11032 | miR-27a | 1.5 | 2315.4 | 1098.7 | 10.6 | 1594.2 |
| 11024 | miR-223 | 1.4 | 968.3 | 565.3 | 9.4 | 690.2 |

TABLE 2
New, downregulated miRNAs in breast cancer compared to normal
breast tissue.
Note the striking downregulation of miR-205.

| | | | raw m | raw m | | |
|---|---|---|---|---|---|---|
| GeneID | miRNA | fold | Tumor | normal | A mean | 2\hat{ }Am |
| 11006 | miR-205 | −39.0 | 156.6 | 4747.2 | 9.7 | 853.3 |
| 11002 | miR-200c | −5.7 | 162.9 | 1209.8 | 8.7 | 429.8 |
| 11001 | miR-200b | −3.2 | 149.1 | 739.9 | 8.3 | 319.2 |
| 10917 | miR-100 | −2.8 | 1131.6 | 2204.7 | 10.6 | 1572.7 |
| 10913 | let-7c | −2.3 | 11062.0 | 15160.9 | 13.7 | 12933.9 |
| 10912 | let-7b | −2.3 | 7535.8 | 10427.7 | 13.1 | 8723.5 |
| 11030 | miR-26a | −2.3 | 3818.7 | 5397.6 | 12.1 | 4535.1 |
| 10935 | miR-130a | −2.2 | 940.2 | 1435.6 | 10.1 | 1134.2 |
| 11031 | miR-26b | −2.2 | 1971.8 | 2806.1 | 11.2 | 2347.6 |
| 10989 | miR-195 | −2.0 | 2209.2 | 2863.0 | 11.3 | 2493.8 |
| 10924 | miR-10a | −2.0 | 564.8 | 996.1 | 9.5 | 731.9 |
| 11059 | miR-326 | −2.0 | 1150.6 | 1629.3 | 10.4 | 1357.4 |
| 10925 | miR-10b | −1.8 | 1762.4 | 2201.6 | 10.9 | 1969.1 |
| 10946 | miR-141 | −1.8 | 110.2 | 433.3 | 7.8 | 216.2 |
| 11049 | miR-30b | −1.8 | 1442.8 | 1837.1 | 10.7 | 1627.9 |
| 10985 | miR-191 | −1.8 | 711.7 | 1007.1 | 9.7 | 841.5 |
| 13148 | miR-195 | −1.8 | 2111.3 | 2515.0 | 11.2 | 2274.6 |
| 4500 | let-7g | −1.7 | 2648.8 | 2954.2 | 11.4 | 2795.7 |
| 13179 | miR-455 | −1.7 | 140.6 | 479.5 | 8.0 | 258.8 |
| 11176 | miR-526b | −1.7 | 96.8 | 394.7 | 7.6 | 194.7 |
| 11183 | miR-99a | −1.6 | 441.7 | 748.5 | 9.2 | 572.2 |
| 11149 | miR-515-5p | −1.6 | 180.6 | 501.8 | 8.2 | 296.1 |
| 13126 | miR-191* | −1.5 | 155.3 | 458.7 | 8.1 | 266.1 |
| 11017 | miR-217 | −1.5 | 135.2 | 425.3 | 7.8 | 229.1 |
| 10958 | miR-150 | −1.5 | 284.6 | 590.9 | 8.6 | 401.6 |
| 11039 | miR-29a | −1.5 | 4099.7 | 3818.5 | 11.9 | 3895.8 |
| 13139 | let-7e | −1.5 | 2285.1 | 2266.8 | 11.1 | 2269.6 |
| 13129 | miR-452* | −1.5 | 294.0 | 577.9 | 8.6 | 394.1 |
| 11054 | miR-320 | −1.5 | 3616.1 | 3306.1 | 11.8 | 3453.7 |

TABLE 3
Differentially expressed miRNAs as reported by Imagene analysis software.

| Annotation | Reference | Experiment | Difference | | Significance |
|---|---|---|---|---|---|
| 11006 | hsa-miR-205 | 11.98 | 7.49 | −4.49 | SIGNIFICANT (DOWN) |
| 11002 | hsa-miR-200c | 9.91 | 7.58 | −2.33 | SIGNIFICANT (DOWN) |
| 11001 | hsa-miR-200b | 9.08 | 7.56 | −1.52 | SIGNIFICANT (DOWN) |
| 10913 | hsa-let-7c | 14.30 | 13.02 | −1.29 | SIGNIFICANT (DOWN) |
| 10912 | hsa-let-7b | 13.72 | 12.46 | −1.26 | SIGNIFICANT (DOWN) |
| 3320 | hsa-let-7a | 14.61 | 13.39 | −1.22 | SIGNIFICANT (DOWN) |
| 10929 | hsa-miR-125b | 13.20 | 12.51 | −0.69 | SIGNIFICANT (DOWN) |
| 10946 | hsa-miR-141 | 8.22 | 7.54 | −0.69 | SIGNIFICANT (DOWN) |
| 10939 | hsa-miR-133b | 8.43 | 7.81 | −0.62 | SIGNIFICANT (DOWN) |
| 11017 | hsa-miR-217 | 8.13 | 7.55 | −0.59 | SIGNIFICANT (DOWN) |
| 10917 | hsa-miR-100 | 10.90 | 10.34 | −0.56 | UNCHANGED |
| 11000 | hsa-miR-200a | 8.53 | 7.99 | −0.54 | UNCHANGED |
| 11176 | hsa-miR-526b | 8.30 | 7.76 | −0.53 | UNCHANGED |
| 11277 | No_known_hsa_target | 8.15 | 7.64 | −0.52 | UNCHANGED |
| 11138 | hsa-miR-506 | 8.59 | 8.07 | −0.51 | UNCHANGED |
| 11051 | hsa-miR-30d | 9.26 | 8.75 | −0.51 | UNCHANGED |
| 10942 | hsa-miR-135b | 7.98 | 7.48 | −0.50 | UNCHANGED |
| 11023 | hsa-miR-222 | 9.89 | 10.52 | 0.64 | SIGNIFICANT (UP) |
| 11048 | hsa-miR-30a-5p | 9.48 | 10.12 | 0.65 | SIGNIFICANT (UP) |
| 11216 | No_known_hsa_target | 11.72 | 12.38 | 0.66 | SIGNIFICANT (UP) |
| 13174 | hsa-miR-30e-5p | 8.27 | 8.94 | 0.67 | SIGNIFICANT (UP) |
| 11082 | hsa-miR-370 | 9.00 | 9.68 | 0.68 | SIGNIFICANT (UP) |
| 11260 | No_known_hsa_target | 8.71 | 9.41 | 0.70 | SIGNIFICANT (UP) |
| 11175 | hsa-miR-525 | 8.11 | 8.88 | 0.78 | SIGNIFICANT (UP) |
| 11235 | No_known_hsa_target | 8.04 | 8.85 | 0.80 | SIGNIFICANT (UP) |
| 10956 | hsa-miR-148b | 8.16 | 8.97 | 0.81 | SIGNIFICANT (UP) |
| 11208 | No_known_hsa_target | 8.63 | 9.43 | 0.81 | SIGNIFICANT (UP) |
| 11069 | hsa-miR-342 | 8.99 | 9.83 | 0.85 | SIGNIFICANT (UP) |
| 13148 | hsa-miR-195 | 10.71 | 11.59 | 0.88 | SIGNIFICANT (UP) |
| 13175 | hsa-miR-27b | 10.58 | 11.47 | 0.89 | SIGNIFICANT (UP) |
| 11059 | hsa-miR-326 | 9.72 | 10.63 | 0.91 | SIGNIFICANT (UP) |
| 4700 | hsa-miR-140 | 8.33 | 9.25 | 0.92 | SIGNIFICANT (UP) |
| 11229 | No_known_hsa_target | 9.92 | 10.86 | 0.94 | SIGNIFICANT (UP) |
| 10306 | hsa-miR-146b | 9.29 | 10.26 | 0.96 | SIGNIFICANT (UP) |
| 11228 | No_known_hsa_target | 7.81 | 8.82 | 1.01 | SIGNIFICANT (UP) |
| 11072 | hsa-miR-34a | 8.13 | 9.16 | 1.03 | SIGNIFICANT (UP) |
| 11259 | No_known_hsa_target | 8.14 | 9.17 | 1.03 | SIGNIFICANT (UP) |
| 11024 | hsa-miR-223 | 9.06 | 10.12 | 1.06 | SIGNIFICANT (UP) |
| 11201 | No_known_hsa_target | 8.83 | 9.90 | 1.07 | SIGNIFICANT (UP) |
| 10989 | hsa-miR-195 | 10.75 | 11.82 | 1.07 | SIGNIFICANT (UP) |
| 4500 | hsa-let-7g | 11.06 | 12.15 | 1.08 | SIGNIFICANT (UP) |
| 11022 | hsa-miR-221 | 9.24 | 10.33 | 1.09 | SIGNIFICANT (UP) |
| 13180 | hsa-miR-483 | 9.66 | 10.76 | 1.10 | SIGNIFICANT (UP) |
| 11050 | hsa-miR-30c | 10.00 | 11.15 | 1.16 | SIGNIFICANT (UP) |
| 5560 | hsa-miR-185 | 8.25 | 9.49 | 1.24 | SIGNIFICANT (UP) |
| 11041 | hsa-miR-29c | 9.44 | 10.69 | 1.25 | SIGNIFICANT (UP) |
| 11035 | hsa-miR-296 | 8.94 | 10.24 | 1.30 | SIGNIFICANT (UP) |
| 13139 | hsa-let-7e | 10.45 | 11.75 | 1.30 | SIGNIFICANT (UP) |
| 6500 | hsa-let-7f | 12.35 | 13.68 | 1.33 | SIGNIFICANT (UP) |
| 10995 | hsa-miR-199a* | 11.67 | 13.20 | 1.54 | SIGNIFICANT (UP) |
| 11135 | hsa-miR-503 | 9.72 | 11.26 | 1.55 | SIGNIFICANT (UP) |
| 11279 | U6-snRNA-2 | 12.05 | 13.61 | 1.56 | SIGNIFICANT (UP) |
| 11220 | No_known_hsa_target | 10.81 | 12.42 | 1.61 | SIGNIFICANT (UP) |
| 10996 | hsa-miR-199b | 9.53 | 11.15 | 1.62 | SIGNIFICANT (UP) |
| 11124 | hsa-miR-492 | 9.18 | 10.82 | 1.64 | SIGNIFICANT (UP) |
| 11115 | hsa-miR-451 | 10.99 | 12.65 | 1.66 | SIGNIFICANT (UP) |
| 5740 | hsa-miR-21 | 12.49 | 14.15 | 1.66 | SIGNIFICANT (UP) |
| 11003 | hsa-miR-202 | 9.58 | 11.29 | 1.71 | SIGNIFICANT (UP) |
| 10934 | hsa-miR-129 | 9.48 | 11.22 | 1.74 | SIGNIFICANT (UP) |
| 11146 | hsa-miR-513 | 10.79 | 12.57 | 1.78 | SIGNIFICANT (UP) |
| 11078 | hsa-miR-365 | 9.10 | 10.89 | 1.79 | SIGNIFICANT (UP) |
| 11020 | hsa-miR-22 | 9.18 | 10.98 | 1.80 | SIGNIFICANT (UP) |
| 11126 | hsa-miR-494 | 11.00 | 12.82 | 1.82 | SIGNIFICANT (UP) |
| 10987 | hsa-miR-193b | 8.14 | 10.00 | 1.86 | SIGNIFICANT (UP) |
| 4610 | hsa-miR-126 | 10.37 | 12.22 | 1.86 | SIGNIFICANT (UP) |
| 10965 | hsa-miR-15a | 8.62 | 10.50 | 1.88 | SIGNIFICANT (UP) |
| 10966 | hsa-miR-15b | 9.58 | 11.48 | 1.90 | SIGNIFICANT (UP) |
| 10915 | hsa-let-7i | 10.62 | 12.52 | 1.91 | SIGNIFICANT (UP) |
| 11026 | hsa-miR-23a | 10.94 | 12.91 | 1.97 | SIGNIFICANT (UP) |
| 11214 | No_known_hsa_target | 8.32 | 10.30 | 1.98 | SIGNIFICANT (UP) |
| 11130 | hsa-miR-498 | 10.24 | 12.24 | 2.00 | SIGNIFICANT (UP) |
| 11205 | No_known_hsa_target | 9.67 | 11.81 | 2.14 | SIGNIFICANT (UP) |
| 11212 | No_known_hsa_target | 10.38 | 12.73 | 2.35 | SIGNIFICANT (UP) |
| 11248 | hsa-miR-451 | 11.55 | 14.01 | 2.47 | SIGNIFICANT (UP) |
| 11032 | hsa-miR-27a | 9.62 | 12.13 | 2.51 | SIGNIFICANT (UP) |
| 10986 | hsa-miR-193a | 8.58 | 11.18 | 2.61 | SIGNIFICANT (UP) |
| 11270 | No_known_hsa_target | 8.88 | 11.51 | 2.63 | SIGNIFICANT (UP) |
| 11014 | hsa-miR-214 | 10.23 | 12.93 | 2.70 | SIGNIFICANT (UP) |
| 10943 | hsa-miR-136 | 8.24 | 11.15 | 2.91 | SIGNIFICANT (UP) |
| 11028 | hsa-miR-24 | 9.77 | 12.68 | 2.92 | SIGNIFICANT (UP) |
| 10994 | hsa-miR-199a | 9.54 | 12.61 | 3.07 | SIGNIFICANT (UP) |
| 11086 | hsa-miR-373* | 9.55 | 12.83 | 3.28 | SIGNIFICANT (UP) |
| 10967 | hsa-miR-16 | 9.64 | 13.02 | 3.37 | SIGNIFICANT (UP) |
| 11278 | U6-snRNA-1 | 9.23 | 12.63 | 3.40 | SIGNIFICANT (UP) |
| 11054 | hsa-miR-320 | 10.03 | 13.50 | 3.47 | SIGNIFICANT (UP) |
| 10947 | hsa-miR-142-3p | 8.40 | 12.23 | 3.83 | SIGNIFICANT (UP) |

A total of 86 out of 398 miRNAs were found to be differentially expressed between breast cancer and normal adjacent tissue. Of new miRNAs identified, 29 were down- and 32 were up-regulated in breast cancer compared to normal.

Of the 4 “No known Hs target” capture probes that gave a high signal in breast cancer vs. normal, the following considerations apply:

| GeneID | miRNA | fold | raw m T | raw m n |
|---|---|---|---|---|
| 10994 | miR-199a | 2.9 | 4104.3 | 980.8 |
| 11205 | Hs target: miR-199a/b 1mm | 2.3 | 3126.3 | 938.2 |

The “unknown” Hs target corresponds to miR-199a with a single mismatch, which is in fine agreement with the perfect match signal from miR-199a.

| GeneID | miRNA | fold | raw m T | raw m n |
|---|---|---|---|---|
| 11086 | miR-373* | 1.8 | 4260.4 | 1629.3 |
| 11212 | Hs target: miR-373* 3mm | 1.6 | 4914.2 | 1948.4 |

Here, the unknown Hs target is miR-373 with 3 mismatches, and again, we see nearly identical signals from the perfect match capture probe 11086 and the non-perfect match probe 11212.

| GeneID | miRNA | fold | raw m T | raw m n |
|---|---|---|---|---|
| 11214 | mmu-miR-291a-5p | 2.2 | 965.9 | 428.8 |
| 11270 | rno-miR-347 | 2.2 | 2070.5 | 680.8 |

These two probes, on the other hand, do not share significant similarity with any known human sequence.

11214 is a murine sequence, 11270 is from rat. The possibility of cross-hybridization cannot be excluded, although no obvious human target sequence could be found.

In conclusion, we see a clear difference in miRNA expression pattern between breast cancer tissue and normal breast.

### Example 2

List of LNA-Substituted Detection Probes for Detection of MicroRNAs Associated with Breast Cancer in Humans.

LNA nucleotides are depicted by capital letters, DNA nucleotides by lowercase letters, C denotes LNA methyl-cytosine. The detection probes can be used to detect and analyze conserved vertebrate miRNAs, such as human miRNAs by RNA in situ hybridization, Northern blot analysis and by silencing using the probes as miRNA inhibitors. The LNA-modified probes can be conjugated with a variety of haptens or fluorochromes for miRNA in situ hybridization using standard methods. 5′-end labeling using T4 polynucleotide kinase and gamma-32P-ATP can be carried out by standard methods for Northern blot analysis. In addition, the LNA-modified probe sequences can be used as capture sequences for expression profiling by LNA oligonucleotide microarrays. Covalent attachment to the solid surfaces of the capture probes can be accomplished by incorporating a NH₂—C₆— or a NH₂—C₆-hexaethylene glycol monomer or dimer group at the 5′-end or at the 3′-end of the probes during synthesis. As disclosed in PCT/DK2005/000838,

it is possible to map miRNA in cells to determine the tissue origin of these cells, the present invention presents a convenient means for detection of tissue origin of tumors.

Hence, the present invention in general relates to a method for determining tissue origin of breast tumors comprising probing cells of the tumor with a collection of probes which is capable of mapping miRNA to a tissue origin.

### Example 3

miRNAs which may originate from more that one precursor:

hsa-let-7a
>hsa-let-7a-1 MI0000060
UGGGAUGAGGUAGUAGGUUGUAUAGUUUUAGGGUCACACCCACCACUGGGAGAUAACUAUACAAUCUACUGUCUU
UCCUA
(SEQ ID 349)
>hsa-let-7a-2 MI0000061
AGGUUGAGGUAGUAGGUUGUAUAGUUUAGAAUUACAUCAAGGGAGAUAACUGUACAGCCUCCUAGCUUUCCU
hsa-let-7f
>hsa-let-7f-1 MI0000067
UCAGAGUGAGGUAGUAGAUUGUAUAGUUGUGGGGUAGUGAUUUUACCCUGUUCAGGAGAUAACUAUACAAUCUAU
UGCCUUCCCUGA
(SEQ ID 350)
>hsa-let-7f-2 MI0000068
UGUGGGAUGAGGUAGUAGAUUGUAUAGUUUUAGGGUCAUACCCCAUCUUGGAGAUAACUAUACAGUCUACUGUCU
UUCCCACG
hsa-mir-9
>hsa-mir-9-1 MI0000466
CGGGGUUGGUUGUUAUCUUUGGUUAUCUAGCUGUAUGAGUGGUGUGGAGUCUUCAUAAAGCUAGAUAACCGAAAG
UAAAAAUAACCCCA
(SEQ ID 351)
>hsa-mir-9-2 MI0000467
GGAAGCGAGUUGUUAUCUUUGGUUAUCUAGCUGUAUGAGUGUAUUGGUCUUCAUAAAGCUAGAUAACCGAAAGUA
AAAACUCCUUCA
(SEQ ID 352)
>hsa-mir-9-3 MI0000468
GGAGGCCCGUUUCUCUCUUUGGUUAUCUAGCUGUAUGAGUGCCACAGAGCCGUCAUAAAGCUAGAUAACCGAAAG
UAGAAAUGAUUCUCA
hsa-mir-16
>hsa-mir-16-1 MI0000070
GUCAGCAGUGCCUUAGCAGCACGUAAAUAUUGGCGUUAAGAUUCUAAAAUUAUCUCCAGUAUUAACUGUGCUGCU
GAAGUAAGGUUGAC
(SEQ ID 353)
>hsa-mir-16-2 MI0000115
GUUCCACUCUAGCAGCACGUAAAUAUUGGCGUAGUGAAAUAUAUAUUAAACACCAAUAUUACUGUGCUGCUUUAG
UGUGAC
hsa-mir-24
>hsa-mir-24-1 MI0000080
CUCCGGUGCCUACUGAGCUGAUAUCAGUUCUCAUUUUACACACUGGCUCAGUUCAGCAGGAACAGGAG
>HSA-mir-24-2 MI0000081
CUCUGCCUCCCGUGCCUACUGAGCUGAAACACAGUUGGUUUGUGUACACUGGCUCAGUUCAGCAGGAACAGGG
(SEQ ID 354)
>hsa-mir-24-2 MI0000081
CUCUGCCUCCCGUGCCUACUGAGCUGAAACACAGUUGGUUUGUGUACACUGGCUCAGUUCAGCAGGAACAGGG
hsa-mir-26a
>hsa-mir-26a-1 MI0000083
GUGGCCUCGUUCAAGUAAUCCAGGAUAGGCUGUGCAGGUCCCAAUGGGCCUAUUCUUGGUUACUUGCACGGGGAC
GC
(SEQ ID 355)
>hsa-mir-26a-2 MI0000750
GGCUGUGGCUGGAUUCAAGUAAUCCAGGAUAGGCUGUUUCCAUCUGUGAGGCCUAUUCUUGAUUACUUGUUUCUG
GAGGCAGCU
hsa-mir-30c
>hsa mir-30c-1 MI0000736
ACCAUGCUGUAGUGUGUGUAAACAUCCUACACUCUCAGCUGUGAGCUCAAGGUGGCUGGGAGAGGGUUGUUUACU
CCUUCUGCCAUGGA
(SEQ ID 356)
>hsa-mir-30c-2 MI0000254
AGAUACUGUAAACAUCCUACACUCUCAGCUGUGGAAAGUAAGAAAGCUGGGAGAAGGCUGUUUACUCUUUCU
hsa-mir-101
>hsa-mir 101-1 MI0000103
UGCCCUGGCUCAGUUAUCACAGUGCUGAUGCUGUCUAUUCUAAAGGUACAGUACUGUGAUAACUGAAGGAUGGCA
(SEQ ID 357)
>hsa-mir-101-2 MI0000739
ACUGUCCUUUUUCGGUUAUCAUGGUACCGAUGCUGUAUAUCUGAAAGGUACAGUACUGUGAUAACUGAAGAAUGG
UGGU
hsa-mir-125b
>hsa-mir-125b-1 MI0000446
UGCGCUCCUCUCAGUCCCUGAGACCCUAACUUGUGAUGUUUACCGUUUAAAUCCACGGGUUAGGCUCUUGGGAGC
UGCGAGUCGUGCU
(SEQ ID 358)
>hsa-mir-125b-2 MI0000470
ACCAGACUUUUCCUAGUCCCUGAGACCCUAACUUGUGAGGUAUUUUAGUAACAUCACAAGUCAGGCUCUUGGGAC
CUAGGCGGAGGGGA
hsa-mir-129
>hsa-mir-129-1 MI0000252
GGAUCUUUUUGCGGUCUGGGCUUGCUGUUCCUCUCAACAGUAGUCAGGAAGCCCUUACCCCAAAAAGUAUCU
(SEQ ID 359)
>hsa-mir-129-2 MI0000473
UGCCCUUCGCGAAUCUUUUUGCGGUCUGGGCUUGCUGUACAUAACUCAAUAGCCGGAAGCCCUUACCCCAAAAAG
CAUUUGCGGAGGGCG
hsa-mir-199a
>hsa-mir-199a-1 MI0000242
GCCAACCCAGUGUUCAGACUACCUGUUCAGGAGGCUCUCAAUGUGUACAGUAGUCUGCACAUUGGUUAGGC
(SEQ ID 360)
>hsa-mir-199a-2 MI0000281
AGGAAGCUUCUGGAGAUCCUGCUCCGUCGCCCCAGUGUUCAGACUACCUGUUCAGGACAAUGCCGUUGUACAGUA
GUCUGCACAUUGGUUAGACUGGGCAAGGGAGAGCA
hsa-mir-365
>hsa-mir-365-1 MI0000767
ACCGCAGGGAAAAUGAGGGACUUUUGGGGGCAGAUGUGUUUCCAUUCCACUAUCAUAAUGCCCCUAAAAAUCCUU
AUUGCUCUUGCA
(SEQ ID 361)
>hsa-mir-365-2 MI0000769
AGAGUGUUCAAGGACAGCAAGAAAAAUGAGGGACUUUCAGGGGCAGCUGUGUUUUCUGACUCAGUCAUAAUGCCC
CUAAAAAUCCUUAUUGUUCUUGCAGUGUGCAUCGGG
hsa-mir-513
>hsa-mir-513-1 MI0003191
GGGAUGCCACAUUCAGCCAUUCAGCGUACAGUGCCUUUCACAGGGAGGUGUCAUUUAUGUGAACUAAAAUAUAAA
UUUCACCUUUCUGAGAAGGGUAAUGUACAGCAUGCACUGCAUAUGUGGUGUCCC
(SEQ ID 362)
>hsa-mir-513-2 MI0003192
GGAUGCCACAUUCAGCCAUUCAGUGUGCAGUGCCUUUCACAGGGAGGUGUCAUUUAUGUGAACUAAAAUAUAAAU
UUCACCUUUCUGAGAAGGGUAAUGUACAGCAUGCACUGCAUAUGUGGUGUCC
hsa-mir-515
>hsa-mir-515-1 MI0003144
UCUCAUGCAGUCAUUCUCCAAAAGAAAGCACUUUCUGUUGUCUGAAAGCAGAGUGCCUUCUUUUGGAGCGUUACU
GUUUGAGA
(SEQ ID 363)
>hsa-mir-515-2 MI0003147
UCUCAUGCAGUCAUUCUCCAAAAGAAAGCACUUUCUGUUGUCUGAAAGCAGAGUGCCUUCUUUUGGAGCGUUACU
GUUUGAGA
hsa-mir-516
>hsa-mir-516-1 MI0003180
UCUCAGGCUGUGACCUUCUCGAGGAAAGAAGCACUUUCUGUUGUCUGAAAGAAAAGAAAGUGCUUCCUUUCAGAG
GGUUACGGUUUGAGA
(SEQ ID 364)
>hsa-mir-516-2 MI0003181
UCUCAGGUUGUGACCUUCUCGAGGAAAGAAGCACUUUCUGUUGUCUGAAAGAAAAGAAAGUGCUUCCUUUCAGAG
GGUUACGGUUUGAGA
(SEQ ID 365)
>hsa-mir-516-3 MI0003167
UCUCAUGAUGUGACCAUCUGGAGGUAAGAAGCACUUUGUGUUUUGUGAAAGAAAGUGCUUCCUUUCAGAGGGUUA
CUCUUUGAGA
(SEQ ID 366)
>hsa-mir-516-4 MI0003172
UCUCAGGCUGUGACCAUCUGGAGGUAAGAAGCACUUUCUGUUUUGUGAAAGAAAAGAAAGUGCUUCCUUUCAGAG
GGUUACUCUUUGAGA
rno-mir-7-1
>rno-mir-7-1 MI0000641
UGUUGGCCUAGUUCUGUGUGGAAGACUAGUGAUUUUGUUGUUUUUAGAUAACUAAGACGACAACAAAUCACAGUC
UGCCAUAUGGCACAGGCCACCU
(SEQ ID 367)
>rno-mir-7-2 MI0000836
GGACAGACCAGCCCUGUCUGGAAGACUAGUGAUUUUGUUGUUGUGUCUGUGUCCAACAACAAGUCCCAGUCUGCC
ACAUGGUGUUGGUCACAUCA

### Example 4

TABLE 4
Summary of results:
Preferred detection probe pairs are designed against each corresponding Pre miRNA SEQ IS and miRNA sequence ID.

| | Non | Breast | | | | | | PROBE SEQUENCE 5′-3′ | |
|---|---|---|---|---|---|---|---|---|---|
| Probe | coding | cancer | | | Pre miRNA | | | Note LNA cytosines preferably | |
| reference | RNA target | expression | SOURCE | | SEQ ID No | Mature RNA seq | miRNA SEQ ID | comprise 5-methyl. | Oligo SEQ ID No |
| 10947 | Has-miR- | UP | HUMAN | SIGNIFICANT | 4 | uguaguguuuccuacuuuaugga | 237 | tCcaTaaAgtAggAaaCacTaca | 114 |
| | 142-3p | | | | | | | | |
| 11248 | Has miR- | UP | HUMAN | SIGNIFICANT | 72 | aaaccguuaccauuacugaguuu | 238 | CtcAgtAatGgtAacGgt | 115 |
| | 451 | | | | | | | | |
| 11115 | Has miR- | UP | HUMAN | SIGNIFICANT | 72 | aaaccguuaccauuacugaguuu | 239 | AaaCtcAgtAatGgtAacGg | 116 |
| | 451 | | | | | | | | |
| 10943 | Has miR- | UP | HUMAN | SIGNIFICANT | 36 | acuccauuuguuuugaugaugga | 240 | tccAtcAtcAaaAcaAatGgaGt | 117 |
| | 136 | | | | | | | | |
| 10986 | Has miR- | UP | HUMAN | SIGNIFICANT | 29 | aacuggccuacaaagucccag | 241 | GggActTtgTagGccAg | 118 |
| | 193a | | | | | | | | |
| 10994 | Has miR- | UP | HUMAN | SIGNIFICANT | 44 | uagguaguuucauguuguugg | 242 | gaAcaGgtAgtCtgAacActGgg | 119 |
| | 199a | | | | | | | | |
| 11278 | U6-snRNA- | UP | HUMAN | SIGNIFICANT | 113 | NR | NR | tCtgTatCgtTccAatTt | 120 |
| | 1 | | | | | | | | |
| 11279 | U6-snRNA- | UP | HUMAN | SIGNIFICANT | 113 | NR | NR | GcgTgtCatCctTgcg | 121 |
| | 2 | | | | | | | | |
| 11124 | Has miR- | UP | HUMAN | SIGNIFICANT | 65 | aggaccugcgggacaagauucuu | 243 | gaAtcTtgTccCgcAggt | 122 |
| | 492 | | | | | | | | |
| 11205 | mmu-miR- | UP | MOUSE | SIGNIFICANT | 76 | cccaguguuuagacuaucuguuc | 244 | gAacAggTagTctAaaCacTg | 123 |
| | 199b | | | | | | | | |
| 10987 | Has miR- | UP | HUMAN | SIGNIFICANT | 12 | aacuggcccucaaagucccgcuuu | 245 | ggActTtgAggGccAgtt | 124 |
| | 193b | | | | | | | | |
| 10995 | Has miR- | UP | HUMAN | SIGNIFICANT | 28 | Cccaguguucagacuaccuguuc | 246 | aacCaaTgtGcaGacTacTgta | 125 |
| | 199a* | | | | | | | | |
| 11214 | mmu-miR- | UP | MOUSE | SIGNIFICANT | 83 | caucaaaguggaggcccucucu | 247 | gGgcCtcCacTttGat | 126 |
| | 291a-5p | | | | | | | | |
| 11078 | Has miR- | UP | HUMAN | SIGNIFICANT | 52 | uaaugccccuaaaaauccuuau | 248 | aTaaGgaTttTtaGggGcaTt | 127 |
| | 365 | | | | | | | | |
| 10965 | Has miR- | UP | HUMAN | SIGNIFICANT | 75 | uagcagcacauaaugguuugug | 249 | cAcaAacCatTatGtgCtgCta | 128 |
| | 15a | | | | | | | | |
| 11270 | rno-miR- | UP | RAT | SIGNIFICANT | 91 | ugucccucugggucgccca | 250 | gGcgAccCagAgg | 129 |
| | 347 | | | | | | | | |
| 11020 | Has miR- | UP | HUMAN | SIGNIFICANT | 9 | aagcugccaguugaagaacugu | 251 | acaGttCttCaaCtgGcaGctt | 130 |
| | 22 | | | | | | | | |
| 4700 | Has miR- | UP | HUMAN | SIGNIFICANT | 85 | agugguuuuacccuaugguag | 252 | ctAccAtaGggTaaAacCact | 131 |
| | 140 | | | | | | | | |
| 13131 | Has miR- | UP | HUMAN | ??? | 92 | ucucuggagggaagcacuuucug | 253 | aGtgCttCccTccAgag | 132 |
| | 518c* | | | | | | | | |
| 11072 | Has miR- | UP | HUMAN | SIGNIFICANT | 26 | uggcagugucuuagcugguuguu | 254 | aaCaaCcaGctAagAcaCtgCca | 133 |
| | 34a | | | | | | | | |
| 10966 | Has miR- | UP | HUMAN | SIGNIFICANT | 14 | uagcagcacaucaugguuuaca | 255 | tgtAaaCcaTgaTgtGctGcta | 134 |
| | 15b | | | | | | | | |
| 11082 | Has miR- | UP | HUMAN | SIGNIFICANT | 46 | gccugcugggguggaaccugg | 256 | ccAggTtcCacCccAgcAggc | 135 |
| | 370 | | | | | | | | |
| 11014 | Has miR- | UP | HUMAN | SIGNIFICANT | 39 | acagcaggcacagacaggcag | 257 | ctGccTgtCtgTgcCtgCtgt | 136 |
| | 214 | | | | | | | | |
| 11175 | Has miR- | UP | HUMAN | SIGNIFICANT | 69 | cuccagagggaugcacuuucu | 258 | AaaGtgCatCccTctGga | 137 |
| | 525 | | | | | | | | |
| 11086 | Has miR- | UP | HUMAN | SIGNIFICANT | 66 | gaagugcuucgauuuuggggugu | 259 | acaCccCaaAatCgaAgcActTc | 138 |
| | 373* | | | | | | | | |
| 10956 | Has miR- | UP | HUMAN | SIGNIFICANT | 6 | ucagugcaucacagaacuuugu | 260 | acaAagTtcTgtGatGcaCtga | 139 |
| | 148b | | | | | | | | |
| 5560 | Has miR- | UP | HUMAN | SIGNIFICANT | 64 | uggagagaaaggcaguuc | 261 | gAacTgcCttTctCtcCa | 140 |
| | 185 | | | | | | | | |
| 11151 | Has miR- | UP | HUMAN | SIGNIFICANTS | 112 | caucuggagguaagaagcacuuu | 262 | agTgcTtcTtaCctCcaGa | 141 |
| | 516-5p | | | | | | | | |
| 11212 | mmu-miR- | UP | MOUSE | SIGNIFICANT | 84 | cucaaacuaugggggcacuuuuu | 263 | AagTgcCccCatAgtTtgA | 142 |
| | 290 | | | | | | | | |
| 11135 | Has miR- | UP | HUMAN | SIGNIFICANT | 93 | uagcagcgggaacaguucugcag | 264 | AacTgtTccCgcTgcTa | 143 |
| | 503 | | | | | | | | |
| 11032 | Has miR- | UP | HUMAN | SIGNIFICANT | 54 | uucacaguggcuaaguuccgc | 265 | gcGgaActTagCcaCtgTgaa | 144 |
| | 27a | | | | | | | | |
| 11024 | Has miR- | UP | HUMAN | SIGNIFICANT | 24 | ugucaguuugucaaauacccc | 266 | GggGtaTttGacAaaCtgAca | 145 |
| | 223 | | | | | | | | |
| 11277 | rno-miR- | | RAT | | 110 | caacaaaucacagucugccaua | 267 | tGgcAgaCtgTgaTttg | 146 |
| | 7* | | | | | | | | |
| 11023 | Has hsa- | UP | HUMAN | SIGNIFICANT | 42 | agcuacaucuggcuacugggucuc | 268 | gaGacCcaGtaGccAgaTgtAgct | 147 |
| | miR-222 | | | | | | | | |
| 11048 | hsa-miR- | UP | HUMAN | SIGNIFICANT | 94 | uguaaacauccucgacuggaag | 269 | cTtcCagTcgAggAtgTttAca | 148 |
| | 30a-5p | | | | | | | | |
| 11216 | mmu-miR- | UP | MOUSE | SIGNIFICANT | 95 | acucaaacugggggcucuuuug | 270 | caAaaGagCccCcaGtt | 149 |
| | 292-5p | | | | | | | | |
| 13174 | hsa-miR- | UP | HUMAN | SIGNIFICANT | 18 | uguaaacauccuugacugga | 271 | tcCagTcaAggAtgTttAca | 150 |
| | 30e-5p | | | | | | | | |
| 11260 | rno-miR- | UP | RAT | SIGNIFICANT | 90 | ucgaggagcucacagucuagua | 272 | acTagActGtgAgcTccTc | 151 |
| | 151* | | | | | | | | |
| 11235 | mmu-miR- | UP | MOUSE | SIGNIFICANT | 87 | ucccugaggagcccuuugagccug | 273 | ctCaaAggGctCctCaQ | 152 |
| | 351 | | | | | | | | |
| 10956 | hsa-miR- | UP | HUMAN | SIGNIFICANT | 6 | ucagugcaucacagaacuuu˜u | 274 | acaAagTtcTgtGatGcaCtga | 153 |
| | 148b | | | | | | | | |
| 11208 | mmu-miR- | UP | MOUSE | SIGNIFICANT | 82 | gcuucuccuggcucuccucccuc | 275 | gGagAgcCagGagAa | 154 |
| | 207 | | | | | | | | |
| 11069 | hsa-miR- | UP | HUMAN | SIGNIFICANT | 23 | ucucacacagaaaucgcacccguc | 276 | gacGggTgcGatTtcTgtGtgAga | 155 |
| | 342 | | | | | | | | |
| 13148 | hsa-miR- | UP | HUMAN | SIGNIFICANT | 55 | uagcagcacagaaauauuggc | 277 | gCcaAtaTttCtgTgcTgcTa | 156 |
| | 195 | | | | | | | | |
| 13175 | hsa-miR- | UP | HUMAN | SIGNIFICANT | 57 | uucacaguggcuaaguucugc | 278 | gcAgaActTagCcaCtgTgaa | 157 |
| | 27b | | | | | | | | |
| 11059 | hsa-miR- | UP | HUMAN | SIGNIFICANT | 33 | ccucugggcccuuccuccag | 279 | ctgGagGaaGggCccAgaGg | 158 |
| | 326 | | | | | | | | |
| 11229 | mmu-miR- | UP | MOUSE | SIGNIFICANT | 88 | ucgaucggucggucggucagu | 280 | AccGacCgaCcgAtc | 159 |
| | 341 | | | | | | | | |
| 10306 | hsa-miR- | UP | HUMAN | SIGNIFICANT | 37 | ugagaacugaauuccauaggcu | 281 | aGccTatGgaAttCagTtcTca | 160 |
| | 146b | | | | | | | | |
| 11228 | mmu-miR- | UP | MOUSE | SIGNIFICANT | 96 | gcaaagcacagggccugcagaga | 282 | gGccCtgTgcTttGc | 161 |
| | 330 MM1 | | | | | | | | |
| 11259 | mmu-miR- | UP | MOUSE | SIGNIFICANT | 97 | cuagacugaggcuccuugagg | 283 | gGagCctCagTctAgt | 162 |
| | 151 MM1 | | | | | | | | |
| 11201 | mmu-miR- | UP | MOUSE | SIGNIFICANT | 85 | uaccacaggguagaaccacgga | 284 | tCcgTggTtcTacCctg | 163 |
| | 140* | | | | | | | | |
| 10989 | hsa-miR- | UP | HUMAN | SIGNIFICANT | 55 | uagcagcacagaaauauuggc | 285 | gCcaAtaTttCtgTgcTgcTa | 164 |
| | 195 | | | | | | | | |
| 4500 | hsa-let-7g | UP | HUMAN | SIGNIFICANT | 53 | gucaguuugucaaauacccc | 286 | aCtgTacAaaCtaCtaCctCa | 165 |
| 11022 | hsa-miR- | UP | HUMAN | SIGNIFICANT | 58 | agcuacauugucugcuggguuuc | 287 | gAaaCccAgcAgaCaaTgtAgct | 166 |
| | 221 | | | | | | | | |
| 13180 | hsa-miR- | UP | HUMAN | SIGNIFICANT | 68 | ucacuccucuccucccgucuucu | 288 | aaGacGggAggAgag | 167 |
| | 483 | | | | | | | | |
| 11050 | hsa-miR- | UP | HUMAN | SIGNIFICANT | 59 | uguaaacauccuacacucucagc | 289 | gCtgAgaGtgTagGatGttTaca | 168 |
| | 30c | | | | | | | | |
| 11041 | hsa-miR- | UP | HUMAN | SIGNIFICANT | 73 | uagcaccauuugaaaucggu | 290 | aCcgAttTcaAatGgtGcta | 169 |
| | 29c | | | | | | | | |
| 11035 | hsa-miR- | UP | HUMAN | SIGNIFICANT | 41 | agggcccccccucaauccugu | 291 | acAggAttGagGggGggCcct | 170 |
| | 296 | | | | | | | | |
| 13139 | hsa-let-7e | UP | HUMAN | SIGNIFICANT | 19 | ugagguaggagguuguauagu | 292 | actAtaCaaCctCctAccTca | 171 |
| 6500 | hsa-let-7f | UP | HUMAN | SIGNIFICANT | 67 | ugagguaguagauuguauaguu | 293 | aaCtaTacAatCtaCtaCctCa | 172 |
| 11220 | mmu-miR- | UP | MOUSE | SIGNIFICANT | 89 | ggcagaggagggcuguucuucc | 294 | AagAacAgcCctCctCtg | 173 |
| | 298 | | | | | | | | |
| 10996 | hsa-miR- | UP | HUMAN | SIGNIFICANT | 76 | cccaguguuuagacuaucuguuc | 295 | gAacAgaTagTctAaaCacTggg | 174 |
| | 199b | | | | | | | | |
| 5740 | hsa-miR- | UP | HUMAN | V. PREF | 45 | uagcuuaucagacugauguuga | 296 | tCaaCatCagTctGatAagCta | 175 |
| 21 | | | | | | | | | |
| 11003 | hsa-miR- | UP | HUMAN | SIGNIFICANT | 63 | agagguauagggcaugggaaaa | 297 | ttTtcCcaTgcCctAtaCct | 176 |
| | 202 | | | | | | | | |
| 10934 | hsa-miR- | UP | HUMAN | SIGNIFICANT | 25 | cuuuuugcggucugggcuugc | 298 | gcAagCccAgaCcgCaaAaag | 177 |
| | 129 | | | | | | | | |
| 11146 | hsa-miR- | UP | HUMAN | SIGNIFICANT | 62 | uucacagggaggugucauuuau | 299 | aaTgaCacCtcCctGtga | 178 |
| | 513 | | | | | | | | |
| 11126 | hsa-miR- | UP | HUMAN | SIGNIFICANT | 21 | ugaaacauacacgggaaaccucuu | 300 | aGagGttTccCgtGtaTg | 179 |
| | 494 | | | | | | | | |
| 4610 | hsa-miR- | UP | HUMAN | SIGNIFICANT | 78 | ucguaccgugaguaauaaugc | 301 | gcAttAttActCacGgtAcga | 180 |
| | 126 | | | | | | | | |
| 10915 | hsa-let-7i | UP | HUMAN | SIGNIFICANT | 13 | ugagguaguaguuugugcugu | 302 | aCagCacAaaCtaCtaCctCa | 181 |
| 11026 | hsa-miR- | UP | HUMAN | SIGNIFICANT | 50 | aucacauugccagggauuucc | 303 | gGaaAtcCctGgcAatGtgAt | 182 |
| | 23a | | | | | | | | |
| 11130 | hsa-miR- | UP | HUMAN | SIGNIFICANT | 3 | uuucaagccagggggcguuuuuc | 304 | gAaaAacGccCccTgg | 183 |
| | 498 | | | | | | | | |
| 11028 | hsa-miR- | UP | HUMAN | SIGNIFICANT | 27 | uggcucaguucagcaggaacag | 305 | cTgtTccTgcTgaActGagCca | 184 |
| | 24 | | | | | | | | |
| 10967 | hsa-miR- | UP | HUMAN | SIGNIFICANT | 10 | uagcagcacguaaauauuggcg | 306 | ccaAtaTttAcgTgcTgcTa | 185 |
| | 16 | | | | | | | | |
| 11054 | hsa-miR- | UP | HUMAN | SIGNIFICANT | 38 | aaaagcuggguugagagggcgaa | 307 | tTcgCccTctCaaCccAgcTttt | 186 |
| | 32 | | | | | | | | |
| 11006 | hsa-miR- | DOWN | HUMAN | V. PREF. | 47 | uccuucauuccaccggagucug | 308 | caGacTccGgtGgaAtgAagGa | 187 |
| | 205 | | | | | | | | |
| 11002 | hsa-miR- | DOWN | HUMAN | SIGNIF | 77 | uaauacugccggguaaugaugg | 309 | ccAtcAttAccCggCagTatTa | 188 |
| | 200c | | | | | | | | |
| 11001 | hsa-miR- | DOWN | HUMAN | SIGNIF | 51 | uaauacugccugguaaugaugac | 310 | cAtcAttAccAggCagTatTaga | 189 |
| | 200b | | | | | | | | |
| 10917 | hsa-miR- | DOWN | HUMAN | | 11 | aacccguagauccgaacuugug | 311 | cacAagTtcGgaTctAcgGgtt | 190 |
| | 100 | | | | | | | | |
| 10913 | hsa-let-7c | DOWN | HUMAN | SIGNIF | 30 | ugagguaguagguuguaugguu | 312 | aaCcaTacAacCtaCtaCctCa | 191 |
| 10912 | hsa-let-7b | DOWN | HUMAN | SIGNIF | 43 | ugagguaguagguugugugguu | 313 | aaCcaCacAacCtaCtaCctCa | 192 |
| 11030 | hsa-miR- | DOWN | HUMAN | | 98 | uucaaguaauccaggauaggc | 314 | gcCtaTccTggAttActTgaa | 193 |
| | 26a | | | | | | | | |
| 10935 | hsa-miR- | DOWN | HUMAN | | 99 | cagugcaauguuaaaagggcau | 315 | aTgcCctrttAacAttGcaCtg | 194 |
| | 130a | | | | | | | | |
| 11031 | hsa-miR- | DOWN | HUMAN | | 56 | uucaaguaauucaggauagguu | 316 | aacCtaTccTgaAttActTgaa | 195 |
| | 26b | | | | | | | | |
| 10989 | hsa-miR- | DOWN | HUMAN | | 55 | uagcagcacagaaauauuggc | 317 | gCcaAtaTttCtgTgcTgcTa | 196 |
| | 195 | | | | | | | | |
| 10924 | hsa-miR | DOWN | HUMAN | | 100 | uacccuguagauccgaauuugug | 318 | cAcaAatTcgGatCtaCagGgta | 197 |
| | 10a | | | | | | | | |
| 11059 | hsa-miR- | DOWN | HUMAN | | 33 | ccucugggcccuuccuccag | 319 | ctgGagGaaGggCccAgaGg | 198 |
| | 326 | | | | | | | | |
| 10925 | hsa-miR- | DOWN | HUMAN | | 101 | uacccuguagaaccgaauuugu | 320 | aCaaAttCggTtcTacAggGta | 199 |
| | 10b | | | | | | | | |
| 10946 | hsa-miR- | DOWN | HUMAN | SIGNIF | 22 | uaacacugucugguaaagaugg | 321 | cCatCttracCagAcaGtgTta | 200 |
| | 141 | | | | | | | | |
| 11049 | hsa-miR- | DOWN | HUMAN | | 102 | uguaaacauccuacacucagcu | 322 | agcTgaGtgTagGatGttTaca | 201 |
| | 30b | | | | | | | | |
| 10985 | hsa-miR- | DOWN | HUMAN | | 103 | caacggaaucccaaaagcagcu | 323 | agcTgcTttTggGatTccGttg | 202 |
| | 191 | | | | | | | | |
| 13148 | hsa-miR- | DOWN | HUMAN | | 55 | uagcagcacagaaauauuggc | 324 | gCcaAtaTttCtgrgcTgcTa | 203 |
| | 195 | | | | | | | | |
| 4500 | hsa-let-7g | DOWN | HUMAN | | 53 | ugagguaguaguuuguacagu | 325 | aCtgTacAaaCtaCtaCctCa | 204 |
| 13179 | hsa-miR- | DOWN | HUMAN | | 104 | uaugugccuuuggacuacaucg | 326 | gAaaAacGccCccTgg | 205 |
| | 455 | | | | | | | | |
| 11176 | hsa-miR- | DOWN | HUMAN | | 56 | cucuugagggaagcacuuucugu | 327 | aAgtGctTccCtcAagAg | 206 |
| | 526b | | | | | | | | |
| 11183 | hsa-miR- | DOWN | HUMAN | | 105 | aacccguagauccgaucuugug | 328 | cacAagAtcGgaTctAcgGgtt | 207 |
| | 99a | | | | | | | | |
| 11149 | hsa-miR- | DOWN | HUMAN | | 111 | uucuccaaaagaaagcacuuucug | 329 | aGtgCttTctTttGgaGa | 208 |
| | 515-5p | | | | | | | | |
| 13126 | hsa-miR- | DOWN | HUMAN | | 103 | gcugcgcuuggauuucgucccc | 330 | agcTgcTttTggGatTccGttg | 209 |
| | 191* | | | | | | | | |
| 11017 | hsa-miR- | DOWN | HUMAN | SIGNIF | 1 | uacugcaucaggaacugauuggau | 331 | atcCaaTcaGttCctGatGcaGta | 210 |
| | 217 | | | | | | | | |
| 10958 | hsa-miR- | DOWN | HUMAN | | 106 | ucucccaacccuuguaccagug | 332 | cacTggTacAagGgtTggGaga | 211 |
| | 150 | | | | | | | | |
| 11039 | hsa-miR- | DOWN | HUMAN | | 107 | uagcaccaucugaaaucgguu | 333 | aaCcgAttTcaGatGgtGcta | 212 |
| | 29a | | | | | | | | |
| 13129 | hsa-miR- | DOWN | HUMAN | | 108 | uguuugcagaggaaacugagac | 334 | tCttTgcAgaTgaGacTga | 213 |
| | 452* | | | | | | | | |
| 11054 | hsa-miR- | DOWN | HUMAN | | 38 | aaaagcuggguugagagggcgaa | 335 | tTcgCccTctCaaCccAgcTttt | 214 |
| | 320 | | | | | | | | |
| 3320 | hsa-let-7a | | HUMAN | SIGNIF | 40 | ugagguaguagguuguauaguu | 336 | aaCtaTacAacCtaCtaCctCa | 215 |
| 10929 | hsa-miR- | | HUMAN | SIGNIF | 48 | ucccugagacccuaacuuguga | 337 | tcaCaaGttAggGtcTcaGgga | 216 |
| | 125b | | | | | | | | |
| 10939 | hsa-miR- | | HUMAN | SIGNIF | 32 | uugguccccuucaaccagcua | 338 | taGctGgtTgaAggGgaCcaa | 217 |
| | 133b | | | | | | | | |
| 11138 | hsa-miR- | | HUMAN | | 109 | uaaggcacccuucugaguaga | 339 | acTcaGaaGggTgcc | 218 |
| | 506 | | | | | | | | |
| | has-miR- | | HUMAN | SIGNIF | 219 | uacaguacugugauaacugaag | 340 | cTtcAgtTatCacAgtActg | 228 |
| | 101 | | | | | | | | |
| | has-miR- | | HUMAN | SIGNIF | 220 | guccaguuuucccaggaaucccuu | 341 | tCctGggAaaActGga | 229 |
| | 145 | | | | | | | | |
| | has-miR- | | HUMAN | SIGNIF | 221 | ucuuugguuaucuagcuguauga | 342 | cAtaCagCtaGatAacCaaAga | 230 |
| | 9 | | | | | | | | |
| | has-miR- | | HUMAN | SIGNIF | 222 | uggagugugacaaugguguuugu | 343 | caCcaTtgTcaCacTccA | 231 |
| | 122a | | | | | | | | |
| | has-miR- | | HUMAN | SIGNIF | 223 | gucaguuugucaaauacccc | 344 | GaaAgaGacCggTtcActG | 232 |
| | 128b | | | | | | | | |
| | has-mir- | | HUMAN | SIGNIF | 224 | ucuggcuccgugucuucacucc | 345 | AgtGaaGacAcgGagC | 233 |
| | 149 | | | | | | | | |
| | has-miR- | | HUMAN | SIGNIF | 225 | ucccugagacccuuuaaccugug | 346 | acAggTtaAagGgtCtcAg | 234 |
| | 125a | | | | | | | | |
| | has-miR- | | HUMAN | SIGNIF | 226 | ugagaugaagcacuguagcuca | 347 | AgcTacA˜tGctTcaTctCa | 235 |
| | 143 | | | | | | | | |
| | has-miR- | | HUMAN | SIGNIF | 227 | acuccauuuguuuugaugaugga | 348 | cCatCatCaaAacAaaTggAg | 236 |
| | 136 | | | | | | | | |

### Example 5

The aim of this example was to validate the microarray findings in the above examples by an independent method (Q RT-PCR) and in an independent patient sample.

Methods

Samples: Two biopsies were obtained from Patient B diagnosed with breast cancer: one biopsy from the primary tumor, and one biopsy from the normal adjacent tissue to the tumor). Please note that patient B is different from the one (“Patient A”) for which the first array analysis (previous examples) was performed.

RNA extraction: (please see the previous examples, the Trizol method was applied)

Microarray miRNA analysis: (please see previous examples)

The design of the microRNA primers and detection probes used in this example were as follows:

| | | First strand | | | |
|---|---|---|---|---|---|
| | | synthesis | | | |
| Templates: | | RT primers: | | | |
| >EQ | | >EQ | | Sequence | SEQ ID |
| >EQ16 | hsa-miR- | >EQ237 | RT_DNA_hsa- | acttttgagggggacacagacctt | 368 |
| 910 | 21 | 44 | miR-21(201) | ctaagttttgagatcaacatc | |
| >EQ22 | hsa-miR- | >EQ251 | RT_DNA_hsa- | acttttgagggggacacagacctt | 369 |
| 371 | 23a | 81 | miR-23a(201) | ctaagttttgagaggaaatc | |
| >EQ22 | hsa-miR- | >EQ237 | RT_DNA_hsa- | acttttgagggggacacagacctt | 370 |
| 374 | 27a | 56 | miR-27a(201) | ctaagttttgagagcggaact | |
| >EQ25 | hsa-miR- | >EQ254 | RT_DNA_hsa- | acttttgagggggacacagacctt | 371 |
| 378 | 32 | 11 | miR-32(201) | ctaagttttgagagcaactta | |
| >EQ27 | hsa-miR- | 27038 | RT_DNA_hsa- | acttttgagggggacacagacctt | 372 |
| 086 | 125b | | miR-125b | ctaagttttgagatcacaagt | |
| >EQ25 | hsa-miR- | >EQ253 | RT_DNA_hsa- | acttttgagggggacacagacctt | 373 |
| 356 | 136 | 93 | miR-136(201) | ctaagttttgagatccatcat | |
| >EQ18 | hsa-let- | >EQ253 | RT_DNA_hsa- | acttttgagggggacacagacctt | 374 |
| 437 | 7b | 85 | let-7b(201) | ctaagttttgagaaaccacac | |
| >EQ16 | hsa-let-7a | >EQ253 | RT_DNA_hsa-let- | acttttgagggggacacagacctt | 375 |
| 898 | | 84 | 7a(201) | ctaagttttgagaaactatac | |

| miR specific forward primer | | | |
|---|---|---|---|
| >EQ | | Sequence | SEQ ID |
| >EQ237 | F_primer_hsa-miR- | aacctcagcctcgctatggttagcttatcagact | 376 |
| 45 | 21(201) | | |
| >EQ251 | F_primer_hsa-miR-23a(201) | aacctcagcctcgctatgggatcacattgccag | 377 |
| 72 | | | |
| >EQ237 | F_primer_hsa-miR- | aacctcagcctcgctatggttcacagtggcta | 378 |
| 57 | 27a(201) | | |
| >EQ254 | F_primer_hsa-miR-hsa-miR- | aacctcagcctcgctatgggtattgcacattac | 379 |
| 44 | 32(201) | | |
| >EQ270 | F_DNA_hsa-miR-125b | aacctcagcctcgctatggtccctgagacc | 380 |
| 22 | | | |
| >EQ254 | F_primer_hsa-miR-hsa-miR- | aacctcagcctcgctatgggactccatttgttt | 381 |
| 24 | 136(201) | | |
| >EQ254 | F_primer_hsa-miR-hsa- | aacctcagcctcgctatgggtgaggtagtaggt | 382 |
| 16 | let-7b(201) | | |
| >EQ254 | F_primer_hsa-miR-hsa-let-7a | aacctcagcctcgctatgggtgaggtagtaggt | 383 |
| 16 | (201) | | |

| miR specific probe | | | |
|---|---|---|---|
| >EQ | | Sequence | SEQ ID |
| >EQ23 | qPCR_Probe_hsa-miR-21 | FITC-aGACTGATgT#Q2z | 384 |
| 758 | | | |
| >EQ251 | hsa-miR-23a-probe-1 | FITC-cAGGGaTTT#Q2z | 385 |
| 55 | | | |
| >EQ23 | qPCR-Probe_hsa-miR-27a | FITC-cTAAgtTCCGC#Q2z | 386 |
| 761 | | | |
| >EQ254 | hsa-miR-32-probe-1 | FITC-tACTAAgTTGC#Q2z | 387 |
| 61 | | | |
| >EQ27 | hsa-miR-125b-probe-1 | FITC-aAGTTAGGG#Q2z | 388 |
| 062 | | | |
| >EQ254 | hsa-miR-136-probe-1 | FITC-tGaTGATGG#Q2z | 389 |
| 62 | | | |
| >EQ18 | hsa-let-7b qPCR-Probe_66° C. | FITC-acCACACAAC#Q2z | 390 |
| 418 | | | |
| >EQ201 | hsa-let-7a_qPcR-Probe2_Q2 | FITC-acTATACAACCT#Q2z | 391 |
| 79 | | | |

| Oligo | | | | | |
|---|---|---|---|---|---|
| id (EQ | | 5′- | | 3′- | |
| No) | Oligonucleotide name | end | Sequence (5′-3′)^{a} | end | SEQ ID |
| 16901 | hsa-miR-145 | | guccaguuuucccaggaaucccuu | | 400 |
| 24021 | RT_DNA_hsa-miR-145 | | acttttgagggggacacagaccttctaagttttg | | 401 |
| | (201) | | agaaagggatt | | |
| 24037 | F_primer_hsa-miR-145 | | aacctcagcctcgctatggggtccagttttccc | | 402 |
| | (201) | | | | |
| 15809 | FP_NM_000201, Amp 1 | | aacctcagcctcgctatgg | | 403 |
| 15810 | RP_NM_000201, Amp 1 | | acttttgagggggacacaga | | 404 |
| 20317 | hsa-miR-145-qPCR- | Fitc | ccAggAATCcCt#Q2 | p | 405 |
| | Probel#Q2 | | | | |

| aLNA (uppercases), DNA/RNA (lowercases), 5 methyl C (C); | | | | |
|---|---|---|---|---|
| Fluorescein (FITC (Glenn Research, | | | | |
| Prod. Id. No. 10-1964)), quencher #Q1 and #Q4 (see below), | | | | |
| z (5-nitroindole (Glenn Research, | | | | |
| Prod. Id. No. 10-1044)), Phosphate (P). | | | | |
| | First strand | | | |
| | Synthesis Target | | | |
| | specific reverse | | | |
| | primer | | | |
| Templates: | >EQ | | Sequence | SEQ ID |
| SNORD24 | 27119 | RP_SNORD24 | atcagcgatcttggtggttt | 392 |
| U6 | 27126 | RP_U6 snoRNA | aggggccatgctaatcttct | 393 |
| snoRNA | | | | |

| Target specific | | | |
|---|---|---|---|
| forward primer | | | |
| >EQ | | | |
| 27106 | FP_SNORD24 | gcagatgatgtaaaagaatatttgc | 394 |
| 27113 | FP_U6 snoRNA | gcttcggcagcacatatactaa | 395 |

| miR specific probe | | | |
|---|---|---|---|
| >EQ | | | |
| 27073 | SNORD24-probe- 1 | aGAGatGgTg#Q2z | 396 |
| 27080 | U6 snoRNA-probe-1 | FITC-aTCGTTCCA#Q2z | 397 |

| QPCR | | | |
|---|---|---|---|
| Universal | | | |
| primers | | Sequence | SEQ ID |
| EQ23931 | FP_NM 000201, Amp 1 | Aacctcagcctcgctatgg | 398 |
| EQ23932 | RP_NM 000201, Amp 1 | Acttttgagggggacacaga | 399 |

The diagnostic probe according to the invention may therefore comprise a fluorescent probe and/or a quencher. The quencher, (#Q), in the contect of the detection probe of the invention, is preferably selected from dark quencher as disclosed in EP Application No. 2004078170.0, in particular compounds selected from 1,4-bis-(3-hydroxy-propylamino)-anthraquinone, 1-(3-(4,4′-dimethoxy-trityloxy)propyl [symbol] amino)-4-(3-hydroxypropylamino)-anthraquinone, 1-(3-(2-cyanoethoxy(diisopropylamino)phosphinoxy)propylamino)-4-(3-(4,4′-dimethoxy-trityloxy)propylamino)-anthraquinone (#Q1), 1,5-bis-(3-hydroxy-propylamino)-anthraquinone, 1-(3-hydroxypropylamino)-5-(3-(4,4′-dimethoxy-trityloxy)propylamino)-anthraquinone, 1-(3-(cyanoethoxy(diisopropylamino)phosphinoxy)propylamino)-5-(3-(4,4′-dimethoxy-trityloxy)propylamino)-anthraquinone (#Q2), 1,4-bis-(4-(2-hydroxyethyl)phenylamino)-anthraquinone, 1-(4-(2-(4,4′-dimethoxy-trityloxy)ethyl)phenylamino)-4-(4-(2-hydroethyl)phenylamino)-anthraquinone, 1-(4-(2-(2-cyanoethoxy(diisopropylamino) [symbol] phosphinoxy)ethyl)phenylamino)-4-(4-(2-(4,4′-dimethoxy-trityloxy)ethyl)phenylamino)-anthraquinone, and 1,8-bis-(3-hydroxy-propylamino)-anthraquinone; or alternatively selected from 6-methyl-Quinizarin, 1,4-bis(3-hydroxypropylamino)-6-methyl [symbol] anthraquinone, 1-(3-(4,4′-dimethoxy-trityloxy) [symbol] propylamino)-4-(3-hydroxy [symbol] propyl [symbol] amino)-6(7)-methyl-anthraquinone, 1-(3-(2-cyanoethoxy(diisopropylamino) [symbol] phosphinoxy) [symbol] propylamino)-4-(3-(4,4′-dimethoxy-trityloxy)propylamino)-6(7)-methyl-anthraquinone, 1,4-bis(4-(2-hydroethyl)phenylamino)-6-methyl-anthraquinone, 1,4-Dihydroxy-2,3-dihydro-6-carboxy-anthraquinone, 1,4-bis(4-methyl-phenylamino)-6-carboxy-anthraquinone, 1,4-bis(4-methyl-phenylamino)-6-(N-(6,7-dihydroxy-4-oxo-heptane-1-yl))carboxamido-anthraquinone, 1,4-bis(4-methyl-phenylamino)-6-(N-(7-dimethoxytrityloxy-6-hydroxy-4-oxo-heptane-1-yl))carboxamido-anthraquinone, 1,4-Bis(4-methyl-phenylamino)-6-(N-(7-(2-cyanoethoxy(diisopropylamino) [symbol] phosphinoxy)-6-hydroxy-4-oxo-heptane-1-yl))carboxamido-anthraquinone, 1,4-bis(propylamino)-6-carboxy-anthraquinone, 1,4-bis(propylamino)-6-(N-(6,7-dihydroxy-4-oxo-heptane-1-yl))carboxamido-anthraquinone, 1,4-bis(propylamino)-6-(N-(7-dimethoxytrityloxy-6-hydroxy-4-oxo-heptane-1-yl))carboxamido-anthraquinone, 1,5-bis(4-(2-hydroethyl) [symbol] phenylamino)-anthraquinone, 1-(4-(2-hydroethyl)phenylamino)-5-(4-(2-(4,4′-dimethoxy-trityloxy)ethyl) [symbol] phenylamino)-anthraquinone, 1-(4-(2-(cyanoethoxy [symbol] (diisopropyl [symbol] amino)phosphinoxy)ethyl) [symbol] phenyl [symbol] amino)-5-(4-(2--(4,4′-dimethoxy-trityloxy)ethyl)phenylamino)-anthraquinone, 1,8-bis(3-hydroxypropylamino)-anthraquinone, 1-(3-hydroxypropylamino)-8-(3-(4,4′-dimethoxy-trityloxy)- [symbol] propylamino)-anthraquinone, 1,8-bis(4-(2-hydroethyl)phenylamino)-anthraquinone, and 1-(4-(2-hydroethyl)phenylamino)-8-(4-(2-(4,4′-dimethoxy-trityloxy)ethyl)phenylamino)-anthraquinone.

PCR Quantification:

Gene Specific First Strand Synthesis of microRNAs and Real-Time Quantitative PCR Detection

1. Gene Specific Priming and Reverse Transcription

The reverse transcription (RT) reaction was performed in 20 μL consisting of 0.5 μg Brain Total RNA template (Ambion, USA) spiked with 100, 10, 1, or 0.1 fmol synthetic miR-145 template, respectively. 1 μM Gene Specific Reverse Transcription Primer (GSP-RT), 1 Incubation buffer (50 mM Tris-HCl, 40 mM KCl, 6 mM MgCl2, 10 mM DTT; pH 8.3 37° C.) (Roche, Germany), 0.5 mM of each of dNTP (Applied Biosystems, USA), 20 U Protector RNase Inhibitor (Roche, Germany), and 40 U M-MuLV reverse transcriptase (Roche, Germany). Three control samples with 0.5 μg Brain total RNA, only, 10 fmol synthetic miR-145 template, and without RNA were included. The RNA templates and the GSP-RT primer were mix and heated 2 min at 95° C. and quenched on ice. The thermocycler DYAD™ (MJ Research DNA engine, USA) was heated to the reaction start temperature. Temperature profile was 30 min 16° C., 30 min 37° C., 5 min 85° C. and cooled down to 4° C., finally. The sample recovered after centrifugation was diluted to five times the originally RT starting volume (100 μL in total).

2. GSP microRNA Real-Time Quantitative PCR Assay Using LNA-Modified Detection Probes.

The real-time PCR reaction (50 μL) was performed in 1 QuantiTect Probe PCR Master Mix (Qiagen, Germany), 400 nM Universal forward primer, 400 nM Universal reverse primer, 80 nM miR-specific forward primer, 200 nM hsa-miR 145-Probe1, 5 μL of the reverse transcription (RT) reaction (described above), and 0.5 U Uracil DNA Glycosylase (Invitrogen, USA). Use the following temperature cycling program was; 10 min at 37° C., 15 min at 95° C., 1 min at 50° C., 39 cycles of 20 s at 94° C. and 1 min at 60° C. The real-time RT-PCR analysis may be performed on a Opticon real-time PCR instrument (MJ Research, USA) or other real-time PCR instruments that are able to detect the FITC fluorophore.

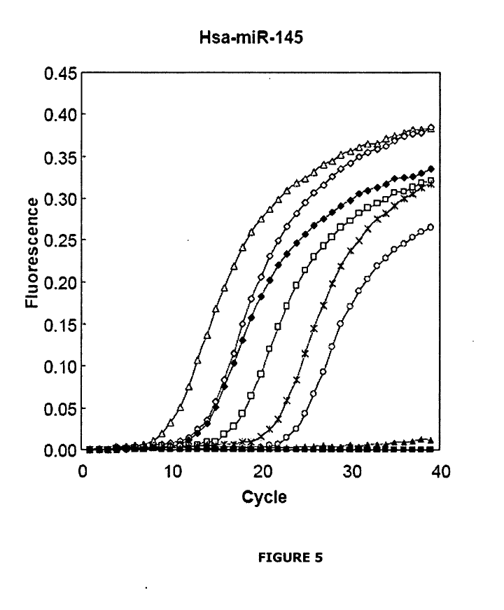

The hsa-miR-145 (acc. no. MIMAT0000437, miRBase, Sanger Institute) RT reactions were subsequently detected using real time PCR as described above, universal PCR primers, miR-specific forward primer, and LNA-modified dual-labelled detection probe for the human miR-145 using a minus template as a negative control. The Ct values using 100, 10, 1, and 0.1 fmol hsa-miR 145 template were 9.2, 12.6, 16.2, and 20.4 for the LNA-modified dual-labelled detection probe (EQ20317), respectively (FIG. 5). The two positive control samples with 0.5 μg Brain total RNA, 10 fmol synthetic miR-145 template gave 23.5 and 12.9, respectively whereas no Ct values were detectable for the negative control experiments (no RNA and no cDNA template).

FIG. 5 shows a dilution series for the human miR-145 real-time quantitative PCR assay. The GSP-RT primer for human miR-145 microRNA was used in first strand synthesis, where the human miR-145 template concentration was 100 (open triangles), 10 (open diamonds), 1 (open squares), or 0.1 fmol (crosses), respectively. The 0.5 μg Brain total RNA is depicted by (open circles), the 10 fmol synthetic miR-145 template by solid diamonds. The negative first strand synthesis without any RNA template is depicted by solid triangles. The cDNA templates were subsequently detected using real-time PCR by the universal PCR primers, the miR-specific forward primer, and the LNA-modified dual-labelled detection probe EQ20317 for the miR-145 microRNA using a minus template as a negative control (solid squares).

Results

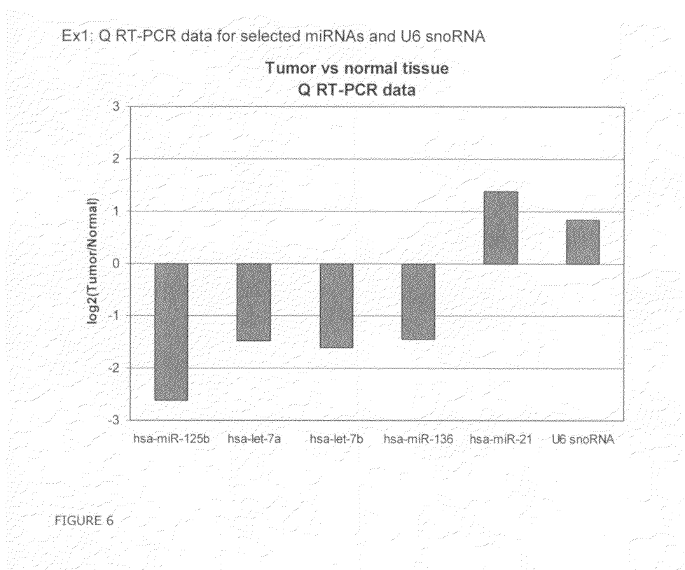

The Q RT-PCR results for a subset of selected RNAs are illustrated shown in FIG. 6. Table 5 compares the PCR data to the microarray data for the corresponding RNA.

TABLE 5
Comparing Q RT-PCR data with microarray data. “up/down” means
that the RNA species is up- or down-regulated in the primary tumor
compared to the normal adjacent tissue

| | Microarray | Q RT-PCR |
|---|---|---|
| RNA | fold regulation | fold regulation |
| miR-21 | 4.9 up | 2.6 up |
| miR-125b | 3.0 down | 6.2 down |
| miR-136 | 1.7 down | 2.7 down |
| let-7a | 1.8 down | 2.8 down |
| let-7b | 1.9 down | 3.0 down |
| U6 snRNA | 2.2 up | 1.8 up |

Conclusion

The Q RT-PCR data for miR-21, miR-125b, let-7a, let-7b, miR-136, and U6 snoRNA were in accord with the miRCURY microarray data. Thus, the original findings have been validated by an independent method.

1. A method for the characterisation of cancer, in a sample derived or obtained from a mammal, preferably a human being, said method comprising the following steps:
a. obtaining at least one test sample, such as a biopsy sample, of a tumor or of a putative tumor, from a patient;
b. presenting a first population of nucleic acid molecules, prepared from said at least one test sample. wherein said first population comprises non-coding RNAs;
c. hybridizing said first population of target molecules, against at least one first detection probe, wherein said at least one first detection probe comprises a recognition sequence derived from a non-coding RNA or precursor thereof;
d. detecting a signal emitted during or subsequent to said hybridization step, said signal providing data which is indicative of hybridization of said at least one first detection probe to a first a non-coding RNA or precursor thereof present within said first population of target molecules;
e. comparing said signal data obtained to reference data, which optionally maybe obtained from said control sample, to provide characterisation of at least one feature of said cancer.

2. A method for the characterisation of cancer, in a sample derived or obtained from a mammal, preferably a human being, said method comprising the following steps:
a. Obtaining at least one test sample, such as a biopsy sample, of a tumor or of a putative tumor, from a patient;
b. Presenting a first population of nucleic acid molecules, prepared from said at least one test sample. wherein said first population comprises small nuclear RNA or miRNA;
c. Hybridizing said first population of target molecules, against at least one first detection probe, wherein said at least one first detection probe comprises a recognition sequence derived from a small nuclear RNA (snRNA) or miRNA or precursor thereof;
d. Detecting a signal emitted during or subsequent to said hybridization step, said signal providing data which is indicative of hybridization of said at least one first detection probe to a first a small nuclear RNA (snRNA) or miRNA or precursor thereof present within said first population of target molecules;
e. Comparing said signal data obtained to reference data, which optionally maybe obtained from said control sample, to provide characterisation of at least one feature of said cancer.

3. The method according to claim 1 or 2, wherein step a) further comprises obtaining at least one control sample; and step b) further comprises presenting a second population of nucleic acid molecules prepared from said control sample, wherein said second population comprises small nucleolar RNA or miRNA; and step c) further comprises hybridizing the second population of nucleic acid molecules to said at least one further detection probe; and step d) further comprises detecting a signal emitted during or subsequent to said hybridization step, said signal providing data which is indicative of hybridization of said at least one further detection probe to a further complementary target within said second population of target molecules; and step e) comprises comparing said signal data obtained from hybridization of the first complementary target to the data obtained from the further complementary target to provide characterisation of at least one feature of said cancer.

4. The method according to any one of claims 1-3, wherein the tumor is selected from the group consisting of: A solid tumor; ovarian cancer, breast cancer, non-small cell lung cancer, renal cell cancer, bladder cancer, esophagus cancer, stomach cancer, prostate cancer, pancreatic cancer, lung cancer, cervical cancer, colon cancer, colorectal cancer, renal cell cancer.

5. The method according to claim 4, wherein the tumor is breast cancer.

6. The method according to any one of claims 1 to 5, wherein the tumor is selected from the group consisting of: a carcinoma, ovarian carcinoma, breast carcinoma, non-small cell lung cancer, renal cell carcinoma, bladder carcinoma, recurrent superficial bladder cancer, stomach carcinoma, prostatic carcinoma, pancreatic carcinoma, lung carcinoma, cervical carcinoma, cervical dysplasia, laryngeal papillomatosis, colon carcinoma, colorectal carcinoma, carcinoid tumors, renal cell carcinoma, a basal cell carcinoma, A malignant melanoma, superficial spreading melanoma, nodular melanoma, lentigo maligna melanoma, acral melagnoma, amelanotic melanoma and desmoplastic melanoma, a sarcoma, osteosarcoma, Ewing's sarcoma, chondrosarcoma, malignant fibrous histiocytoma, fibrosarcoma and Kaposi's sarcoma, glioma.

7. The method according to claim 6, wherein the tumor is a breast carcinoma.

8. The method according to any one of claims 1-7 wherein the tumor is a cancer.

9. The method according to any one of claims 1 to 8, wherein the small nuclear RNA is a small nucleolar RNA, such as U6 snRNA, or precursor of a small nucleolar RNA.

10. The method according to claim 9, wherein the small nucleolar RNA is selected from the group consisting of: SEQ ID NO 113, precursors of SEQ ID NO 113, allelic variants of SEQ ID 113.

11. The method according to any one of claims 1-10, wherein the at least one first detection probe is derived from, or are capable of selectively hybridizing to a region of the small nucleolar RNA.

12. The method according to any one of claims 1-11, wherein the first detection probe is an oligonucleotides which comprises of at least 8 consecutive nucleobase units which are complementary to a region of the small nucleolar RNA with the proviso that there may be no more than a single mismatch between the 8 consecutive nucleobase units of the first detection probe and the region of the small nucleolar RNA.

13. The method according to any one of claims 1-12 wherein said first and second populations of nucleic acids are RNA fractions which further comprise non coding RNA selected from the group consisting of microRNA (miRNA), siRNA piRNA, and precursors therof.

14. The method according to claim 13, wherein said first and second populations of nucleic acids are RNA fractions which further comprise microRNA, and wherein said at least one further complementary target is a microRNA or precursor thereof.

15. The method according to claim 13 or 14, wherein step c) comprises hybridizing said populations of target molecules, against at least one further detection probe, wherein said at least one detection probe comprises a recognition sequence from a microRNA sequence or precursor thereof.

16. The method according to claim 15, wherein the at least one further detection probe is derived from, or are capable of selectively hybridizing to a region of a microRNA.

17. The method according to claim 15 or 16, wherein the at least one further detection probe is an oligonucleotides which comprises of at least 8 consecutive nucleobase units which are complementary to a region of a microRNA, with the proviso that there may be no more than a single mismatch between the 8 consecutive nucleobase units of the first detection probe and the region of the microRNA.

18. The method according to any one of claims 1-17, wherein microRNA is selected from the group consisting of: has-miR-142-3p; has miR-451; has miR-136; has miR-193a; has miR-199a; has miR-492; mmu-miR-199b; has miR-193b; has miR-199a*; mmu-miR-291a-5p; has miR-365; has miR-15a; rno-miR-347; has miR-22; has miR-140; has miR-518c*; has miR-34a; has miR-15b; has miR-370; has miR-214; has miR-525; has miR-373*; has miR-148b; has miR-185; has miR-516-5p; mmu-miR-290; has miR-503; has miR-27a; has miR-223; rno-miR-7*; has hsa-miR-222; hsa-miR-30a-5p; mmu-miR-292-5p; hsa-miR-30e-5p; rno-miR-151*;mmu-miR-351; hsa-miR-148b; mmu-miR-207; hsa-miR-342; hsa-miR-195; hsa-miR-27b; hsa-miR-326; mmu-miR-341; hsa-miR-146b; mmu-miR-330_MM1; mmu-miR-151₁₃ MM1; mmu-miR-140*; hsa-miR-195; hsa-let-7g; hsa-miR-221; hsa-miR-483; hsa-miR-30c; hsa-miR-29c; hsa-miR-296; hsa-let-7e; hsa-let-7f; mmu-miR-298; hsa-miR-199b; hsa-miR-21; hsa-miR-202; hsa-miR-129; hsa-miR-513; hsa-miR-494; hsa-miR-126; hsa- let-7i; hsa-miR-23a; hsa-miR-498; hsa-miR-24; hsa-miR-16; hsa-miR-320; hsa-miR-205; hsa-miR-200c; hsa-miR-200b; hsa-miR-100; hsa-let-7c; hsa-let-7b; hsa-miR-26a; hsa-miR-130a; hsa-miR-26b; hsa-miR-195; hsa-miR-10a; hsa-miR-326; hsa-miR-10b; hsa-miR-141; hsa-miR-30b; hsa-miR-191; hsa-miR-195; hsa-let-7g; hsa-miR-455; hsa-miR-526b; hsa-miR-99a; hsa-miR-515-5p; hsa-miR-191*; hsa-miR-217; hsa-miR-150; hsa-miR-29a; hsa-miR-452*; hsa-miR-320; hsa-let-7a; hsa-miR-125b; hsa-miR-133b; hsa-miR-506; has-miR-101; has-miR-145; has-miR-9; has-miR-122a; has-miR-128b; has-mir-149; has-miR-125a; has-miR-143; has-miR-136 and allelic variants and precursors thereof.

19. The method according to claim 18, wherein the miRNA sequences are selected from the group consisting of: has-miR-21, has-miR-125b, has-let-7a, has-let-7b, and has-miR-143; and allelic variants and precursors thereof.

20. The method according to any one of claims 8-19, wherein step c) comprises hybridizing said populations of target molecule, against at least one, such as at least five, further detection probes, wherein said at least one further detection probe such as at least five, further detection probes, comprises a recognition sequence from a microRNA sequence.

21. The method according to claim 20, wherein the microRNA sequences are selected from the groups as defined in claims 18 or 19.

22. The method according to any of claims 8-21 wherein the at least one feature of said cancer is selected from one or more of the group consisting of: presence or absence of said cancer; type of said cancer ; origin of said cancer; diagnosis of cancer; prognosis of said cancer; therapy outcome prediction; therapy outcome monitoring; suitability of said cancer to treatment, such as suitability of said cancer to chemotherapy treatment and/or radiotherapy treatment; suitability of said cancer to hormone treatment; suitability of said cancer for removal by invasive surgery; suitability of said cancer to combined adjuvant therapy.

23. The method according to any one of claims 1-22, wherein the detection probes are oligonucleotides which comprises at least one nucleotide analogue unit, selected form the group consisting of: 2′-O-alkyl-RNA unit, 2′-OMe-RNA unit, 2′-amino-DNA unit, 2′-fluoro-DNA unit, LNA unit, PNA unit, HNA unit, INA unit.

24. The method according to claim 23, wherein the at least one nucleotide analogue unit is a locked nucleic acid (LNA).

25. The method according to any one of claims 1-24, wherein said oligonucleotide(s) comprises between 8 and 23 nucleobase units.

26. The method according to claim 25, wherein the oligonucleotide(s) comprises between 8 and 16 nucleobase units.

27. The method according to any one of claims 1-26, wherein the oligonucleotide(s) comprises nucleotide analogues inserted with regular spacing between said nucleoside analogues at a frequency selected from the group consisting of at every second nucleotide position, every third nucleotide position, or every fourth nucleotide position, with the proviso that the first and last nucleobases may be either a nucleotide analogue or a nucleotide unit.

28. The method according to any one of claims 24-27, where all the nucleotide analogues present in the oligonucleotide(s) are LNA units.

29. The method according to any one of claims 1-28, wherein the detection probe or probes are capable of selectively hybridizing to the precursor form of the non-coding RNA, but are not capable of selectively hybridizing to the mature form of the non-coding RNA.

30. The method according to claim any one of claims 1-29, wherein the at least first detection probe has a sequence selected from SEQ ID No 120 or SEQ ID NO 121, or a subsequence of at least 8 nucleobases thereof.

31. The method according to any one of claims 1-30, wherein there are at least two first detection probes.

32. The method according to claim 31, wherein the at least two first detection probes comprise one detection probes that has sequence SEQ ID No 120 or a subsequence of at least 8 nucleobases thereof, and one detection probe that has SEQ ID NO 121 or a subsequence of at least 8 nucleobases thereof.

33. The method according to any one of claims 1-32, wherein the one or more further detection probes are oligonucleotides selected from the group comprising: SEQ ID No. 114, SEQ ID No. 115, SEQ ID No. 116, SEQ ID No. 117, SEQ ID No. 118, SEQ ID No. 119, SEQ ID No., SEQ ID No. 123, SEQ ID No. 124, SEQ ID No. 125, SEQ ID No. 126, SEQ ID No. 127, SEQ ID No. 128, SEQ ID No. 129, SEQ ID No. 130, SEQ ID No. 131, SEQ ID No. 132, SEQ ID No. 133, SEQ ID No. 134, SEQ ID No. 135, SEQ ID No. 136, SEQ ID No. 137, SEQ ID No. 138, SEQ ID No. 139, SEQ ID No. 140, SEQ ID No. 141, SEQ ID No. 142, SEQ ID No. 143, SEQ ID No. 144, SEQ ID No. 145, SEQ ID No. 147, SEQ ID No. 148, SEQ ID No. 149, SEQ ID No. 150, SEQ ID No. 151, SEQ ID No. 152, SEQ ID No. 153, SEQ ID No. 154, SEQ ID No. 155, SEQ ID No. 156, SEQ ID No. 157, SEQ ID No. 158, SEQ ID No. 159, SEQ ID No. 160, SEQ ID No. 161, SEQ ID No. 162, SEQ ID No. 163, SEQ ID No. 164, SEQ ID No. 165, SEQ ID No. 166, SEQ ID No. 167, SEQ ID No. 168, SEQ ID No. 169, SEQ ID No. 170, SEQ ID No. 171, SEQ ID No. 172, SEQ ID No. 173, SEQ ID No. 174, SEQ ID No. 175, SEQ ID No. 176, SEQ ID No. 177, SEQ ID No. 178, SEQ ID No. 179, SEQ ID No. 180, SEQ ID No. 181, SEQ ID No. 182, SEQ ID No. 183, SEQ ID No. 184, SEQ ID No. 185, SEQ ID No. 186, SEQ ID No. 187, SEQ ID No. 188, SEQ ID No. 189, SEQ ID No. 190, SEQ ID No. 191, SEQ ID No. 192, SEQ ID No. 193, SEQ ID No. 194, SEQ ID No. 195, SEQ ID No. 196, SEQ ID No. 197, SEQ ID No. 198, SEQ ID No. 199, SEQ ID No. 200, SEQ ID No. 201, SEQ ID No. 202, SEQ ID No. 203, SEQ ID No. 204, SEQ ID No. 205, SEQ ID No. 206, SEQ ID No. 207, SEQ ID No. 208, SEQ ID No. 209, SEQ ID No. 210, SEQ ID No. 211, SEQ ID No. 212, SEQ ID No. 213, SEQ ID No. 214, SEQ ID No. 215, SEQ ID No. 216, SEQ ID No. 217, SEQ ID No. 218; SEQ ID No 228; SEQ ID No 229; SEQ ID No 230; SEQ ID No 231; SEQ ID No 232; SEQ ID No 233; SEQ ID No 234; SEQ ID No 235; and SEQ ID No 236; and variants, homologues and fragments thereof.

34. The method according to claim 33, wherein the one or more further detection probe oligonucleotides are selected from the group comprising: SEQ ID 175; SEQ ID NO 192; SEQ ID NO 216; SEQ ID NO 235; SEQ ID NO 215 and variants, homologues and fragments thereof.

35. The method according to any one of claims 1-34, wherein the at least one control sample is obtained from the same patient.

36. The method according to claim 35, wherein the at least one control sample is obtained from tissue adjacent to said putative tumor, and/or from an equivalent position elsewhere in the body.

37. The method according to any one of claims 1-36, wherein the at least one control sample is obtained from a non tumorous tissue.

38. The method according to any one of claims 1 to 36, wherein the at least one control sample is obtained from a tumor tissue.

39. The method according to any one of claims 35-38, where at least two control samples are obtained, one control sample being obtained from said patient, and at least one further control sample being obtained from a previously obtained sample of a cancer, which may originate from the same patient or a different patient.

40. The method according to any one of claims 1-39, wherein the test and control samples are hybridized to said at least one detection probe simultaneously, either in parallel hybridizations or in the same hybridization experiment.

41. The method according to any one of claims 1-40, wherein the test and control sample or samples are hybridized to said at least one detection probe sequentially, either in the same hybridization experiment, or different hybridization experiments.

42. The method according to any of claims 1-41, wherein an additional step is performed prior to step c), said step comprising performing quantitative analysis of the RNA population obtained from said test sample, and optionally from said control sample or samples.

43. The method according to any one of claims 40-42, wherein the hybridization step in step c) occurs in silico, for example by virtual hybridization.

44. The method according to any one of claims 40-43, wherein the hybridization step is performed by via quantative analysis of the target non-coding RNAs present in said test sample and comparison to equivalent quantitative analysis performed on said one or more control samples.

45. The method according to any of claims 1-44, wherein the hybridization step c) is performed against a collection of said detection probes, said collection of detection probes comprising at least 5 detection probes.

46. The method according to claim 45, wherein the hybridization step is performed against a collection of detection probes comprising least 30 detection probes.

47. The method according to any one of claims 1-46, wherein the hybridization step is performed against an oligonucleotide array.

48. The method according to any one of claims 1 to 46, wherein the hybridization occurs in situ, in or on the biopsy samples

49. The method according to any one of claims 1 to 46, wherein the detection probe or each member of said collection of collection of detection probes are linked to a bead, and wherein said detection of hybridization occurs via bead based detection.

50. The method according to any one claims 1-49, wherein the hybridization step comprises a polymerase chain reaction (PCR).

51. The method according to claim 50, wherein said PCR comprises q-PCR and/or real time PCR (RT-PCR).

52. The method according to any one of claims 1 to 51, wherein the hybridization steps comprises northern blotting.

53. The method according to any one of claims 1 to 47, wherein the hybridization steps comprises an RNase protection assay (RPA).

54. Use of at least one detection probe which comprises a recognition sequence derived from a small nuclear RNA (snRNA) or precursor thereof for the characterisation of cancer.

55. A collection of detection probes, wherein each member of said collection comprises a recognition sequence consisting of nucleobases and/or affinity enhancing nucleobase analogues, wherein said collection of detection probes comprises at least one detection probe according to claim 11 or 12 and at least one detection probe according to any one of claims 16-19.

56. A kit for the detection of cancer, said kit comprising at least one detection probe according to claim 11 or 12.

57. The kit for the detection of cancer according to claim 56, wherein said kit comprises a collection of detection probes according to claim 55.

58. The kit for the detection of cancer according to claims 56 or 57, wherein said kit is in the form or comprises an oligonucleotide array.

59. A method of for the treatment of cancer, said method comprising
a. Isolating at least one tissue sample from a patient suffering from cancer;
b. Performing the characterisation of the at least one tissue sample according to claims any one of claims 1 to 53 and/or utilising the collection of detection probes according to claim 55 or the kit according to any one of claims 56 to 58, to identify at least one feature of said cancer;
c. Based on the at least one feature identified in step b) diagnosing the physiological status of the cancer disease in said patient;
d. Selecting an appropriate form of therapy for said patient based on the said diagnosis;
e. Administering said appropriate form of therapy.

60. The method of for the treatment of cancer according to claim 59, wherein the at least one feature of said cancer is selected from one or more of the group consisting of: Presence or absence of said cancer; type of said cancer; origin of said cancer; diagnosis of cancer; prognosis of said cancer; therapy outcome prediction; therapy outcome monitoring; suitability of said cancer to treatment, such as suitability of said cancer to chemotherapy treatment and/or radiotherapy treatment; suitability of said cancer to hormone treatment; suitability of said cancer for removal by invasive surgery; suitability of said cancer to combined adjuvant therapy.

61. The method of for the treatment of cancer according to claim 60, wherein the at least one feature of said cancer is determination of the origin of said cancer, wherein said cancer is a metestasis and/or a secondary cancer which is remote from the cancer of origin, such as the primary cancer.

62. The method for the treatment of cancer according to any one of claims 59-61, wherein the treatment comprises one or more of the therapies selected from the group consisting of: chemotherapy; hormone treatment; invasive surgery; radiotherapy; and adjuvant systemic therapy.

63. A method for the determination of suitability of a cancer patient for treatment comprising:
a. Isolating at least one tissue sample from a patient suffering from cancer;
b. Performing the characterisation of the at least one tissue sample according to claims any one of claims 1 to 53 and/or utilising the collection of detection probes according to claim 55 or the kit according to any one of claims 56 to 58, to identify at least one feature of said cancer;
c. Based on the at least one feature identified in step b) diagnosing the physiological status of the patient;
d. Based on the said diagnosis obtained in step c) determining whether said patient would benefit from treatment of said cancer.

64. The method of for the determination of suitability of a cancer for treatment according to claim 63, wherein the at least one feature of said cancer is selected from one or more of the group consisting of: Presence or absence of said cancer; type of said cancer ; origin of said cancer; diagnosis of cancer; prognosis of said cancer; therapy outcome prediction; therapy outcome monitoring; suitability of said cancer to treatment, such as suitability of said cancer to chemotherapy treatment and/or radiotherapy treatment; suitability of said cancer to hormone treatment; suitability of said cancer for removal by invasive surgery; suitability of said cancer to combined adjuvant therapy.

65. The method of for the determination of suitability of a cancer for treatment according to claim 63, wherein the at least one feature of said cancer is determination of the origin of said cancer, wherein said cancer is a metastasis and/or a secondary cancer which is remote from the cancer of origin, such as the primary cancer.

66. A method according for the determination of the origin of a metastatic cancer, or a cancer suspected of being a metastasis, comprising:
a. Isolating at least one tissue sample of a metastatic cancer, or a cancer suspected of being a metastasis, from a patient;
b. Performing the characterisation of the at least one tissue sample according to claims any one of claims 1 to 53 and/or utilising the collection of detection probes according to claim 55 or the kit according to any one of claims 56 to 58, to identify the origin of said metastatic cancer.

67. A method for the determination of the origin of a metastatic cancer, or a cancer suspected of being a metastasis, according to claim 66, wherein said characterisation comprises comparison of the at least on feature with the equivalent at least one feature obtained from at least one control sample, wherein said control sample is derived from a cancer of known physiological origin.

68. A method for the determination of the likely prognosis of a cancer patient comprising:
a. Isolating at least one tissue sample from a patient suffering from cancer;
b. Performing the characterisation of the at least one tissue sample according to claims any one of claims 1 to 53 and/or utilising the collection of detection probes according to claim 55 or the kit according to any one of claims 56 to 58, to identify at least one feature of said cancer;
c. wherein said feature allows for the determination of the likely prognosis of said cancer patient.

69. A method for specific isolation, purification, amplification, detection, identification, quantification, inhibition or capture of a target nucleotide sequence in a sample from a cancer, said method comprising contacting said sample with a detection probe as defined in any one of claims 1 to 53 under conditions that facilitate hybridization between said member/probe and said target nucleotide sequence, wherein said target nucleotide sequence is, or is derived from a snRNA associated with cancer.


SEQUENCE LISTING (405 sequences)

SEQ ID NO: 1 - RNA, 110 nt, Homo sapiens
aguauaauuauuacauaguuuuugaugucgcagauacugcaucaggaacugauuggauaagaaucagucaccaucaguuccuaaugcauugccuucagcaucuaaacaag

SEQ ID NO: 2 - RNA, 100 nt, Homo sapiens
ugugucucucucuguguccugccagugguuuuacccuaugguagguuacgucaugcuguucuaccacaggguagaaccacggacaggauaccggggcacc

SEQ ID NO: 3 - RNA, 124 nt, Homo sapiens
aacccuccuugggaagugaagcucaggcugugauuucaagccagggggcguuuuucuauaacuggaugaaaagcaccuccagagcuugaagcucacaguuugagagcaaucgucuaaggaaguu

SEQ ID NO: 4 - RNA, 87 nt, Homo sapiens
gacagugcagucacccauaaaguagaaagcacuacuaacagcacuggaggguguaguguuuccuacuuuauggaugaguguacugug

SEQ ID NO: 5 - RNA, 74 nt, Homo sapiens
gggugagguaguagguuguauaguuuggggcucugcccugcuaugggauaacuauacaaucuacugucuuuccu

SEQ ID NO: 6 - RNA, 99 nt, Homo sapiens
caagcacgauuagcauuugaggugaaguucuguuauacacucaggcuguggcucucugaaagucagugcaucacagaacuuugucucgaaagcuuucua

SEQ ID NO: 7 - RNA, 124 nt, Homo sapiens
gccaccaccaucagccauacuauguguagugccuuauucaggaagguguuacuuaauagauuaauauuuguaaggcacccuucugaguagaguaaugugcaacauggacaacauuugugguggc

SEQ ID NO: 8 - RNA, 71 nt, Homo sapiens
gcgacuguaaacauccucgacuggaagcugugaagccacagaugggcuuucagucggauguuugcagcugc

SEQ ID NO: 9 - RNA, 85 nt, Homo sapiens
ggcugagccgcaguaguucuucaguggcaagcuuuauguccugacccagcuaaagcugccaguugaagaacuguugcccucugcc

SEQ ID NO: 10 - RNA, 81 nt, Homo sapiens
guuccacucuagcagcacguaaauauuggcguagugaaauauauauuaaacaccaauauuacugugcugcuuuagugugac

SEQ ID NO: 11 - RNA, 80 nt, Homo sapiens
ccuguugccacaaacccguagauccgaacuugugguauuaguccgcacaagcuuguaucuauagguaugugucuguuagg

SEQ ID NO: 12 - RNA, 83 nt, Homo sapiens
guggucucagaaucgggguuuugagggcgagaugaguuuauguuuuauccaacuggcccucaaagucccgcuuuuggggucau

SEQ ID NO: 13 - RNA, 84 nt, Homo sapiens
cuggcugagguaguaguuugugcuguuggucggguugugacauugcccgcuguggagauaacugcgcaagcuacugccuugcua

SEQ ID NO: 14 - RNA, 98 nt, Homo sapiens
uugaggccuuaaaguacuguagcagcacaucaugguuuacaugcuacagucaagaugcgaaucauuauuugcugcucuagaaauuuaaggaaauucau

SEQ ID NO: 15 - RNA, 83 nt, Homo sapiens
ugugggaugagguaguagauuguauaguuuuagggucauaccccaucuuggagauaacuauacagucuacugucuuucccacg

SEQ ID NO: 16 - RNA, 89 nt, Homo sapiens
gucagcagugccuuagcagcacguaaauauuggcguuaagauucuaaaauuaucuccaguauuaacugugcugcugaaguaagguugac

SEQ ID NO: 17 - RNA, 86 nt, Homo sapiens
ugcucccccucucuaauccuugcuaucugggugcuagugcuggcucaaugcaaugcaccugggcaaggauucagagagggggagcu

SEQ ID NO: 18 - RNA, 92 nt, Homo sapiens
gggcagucuuugcuacuguaaacauccuugacuggaagcuguaagguguucagaggagcuuucagucggauguuuacagcggcaggcugcca

SEQ ID NO: 19 - RNA, 79 nt, Homo sapiens
cccgggcugagguaggagguuguauaguugaggaggacacccaaggagaucacuauacggccuccuagcuuuccccagg

SEQ ID NO: 20 - RNA, 88 nt, Homo sapiens
ugcgcuccucucagucccugagacccuaacuugugauguuuaccguuuaaauccacggguuaggcucuugggagcugcgagucgugcu

SEQ ID NO: 21 - RNA, 81 nt, Homo sapiens
gauacucgaaggagagguuguccguguugucuucucuuuauuuaugaugaaacauacacgggaaaccucuuuuuuaguauc

SEQ ID NO: 22 - RNA, 95 nt, Homo sapiens
cggccggcccuggguccaucuuccaguacaguguuggauggucuaauugugaagcuccuaacacugucugguaaagauggcucccggguggguuc

SEQ ID NO: 23 - RNA, 99 nt, Homo sapiens
gaaacugggcucaaggugaggggugcuaucugugauugagggacaugguuaauggaauugucucacacagaaaucgcacccgucaccuuggccuacuua

SEQ ID NO: 24 - RNA, 110 nt, Homo sapiens
ccuggccuccugcagugccacgcuccguguauuugacaagcugaguuggacacuccaugugguagagugucaguuugucaaauaccccaagugcggcacaugcuuaccag

SEQ ID NO: 25 - RNA, 72 nt, Homo sapiens
ggaucuuuuugcggucugggcuugcuguuccucucaacaguagucaggaagcccuuaccccaaaaaguaucu

SEQ ID NO: 26 - RNA, 110 nt, Homo sapiens
ggccagcugugaguguuucuuuggcagugucuuagcugguuguugugagcaauaguaaggaagcaaucagcaaguauacugcccuagaagugcugcacguuguggggccc

SEQ ID NO: 27 - RNA, 73 nt, Homo sapiens
cucugccucccgugccuacugagcugaaacacaguugguuuguguacacuggcucaguucagcaggaacaggg

SEQ ID NO: 28 - RNA, 71 nt, Homo sapiens
gccaacccaguguucagacuaccuguucaggaggcucucaauguguacaguagucugcacauugguuaggc

SEQ ID NO: 29 - RNA, 88 nt, Homo sapiens
cgaggaugggagcugagggcugggucuuugcgggcgagaugagggugucggaucaacuggccuacaaagucccaguucucggcccccg

SEQ ID NO: 30 - RNA, 84 nt, Homo sapiens
gcauccggguugagguaguagguuguaugguuuagaguuacacccugggaguuaacuguacaaccuucuagcuuuccuuggagc

SEQ ID NO: 31 - RNA, 90 nt, Homo sapiens
ugcccuucgcgaaucuuuuugcggucugggcuugcuguacauaacucaauagccggaagcccuuaccccaaaaagcauuugcggagggcg

SEQ ID NO: 32 - RNA, 119 nt, Homo sapiens
ccucagaagaaagaugcccccugcucuggcuggucaaacggaaccaaguccgucuuccugagagguuugguccccuucaaccagcuacagcagggcuggcaaugcccaguccuuggaga

SEQ ID NO: 33 - RNA, 95 nt, Homo sapiens
cucaucugucuguugggcuggaggcagggccuuugugaaggcggguggugcucagaucgccucugggcccuuccuccagccccgaggcggauuca

SEQ ID NO: 34 - RNA, 90 nt, Homo sapiens
ccgggccccugugagcaucuuaccggacagugcuggauuucccagcuugacucuaacacugucugguaacgauguucaaaggugacccgc

SEQ ID NO: 35 - RNA, 68 nt, Homo sapiens
cuccggugccuacugagcugauaucaguucucauuuuacacacuggcucaguucagcaggaacaggag

SEQ ID NO: 36 - RNA, 82 nt, Homo sapiens
ugagcccucggaggacuccauuuguuuugaugauggauucuuaugcuccaucaucgucucaaaugagucuucagaggguucu

SEQ ID NO: 37 - RNA, 73 nt, Homo sapiens
ccuggcacugagaacugaauuccauaggcugugagcucuagcaaugcccuguggacucaguucuggugcccgg

SEQ ID NO: 38 - RNA, 82 nt, Homo sapiens
gcuucgcuccccuccgccuucucuucccgguucuucccggagucgggaaaagcuggguugagagggcgaaaaaggaugaggu

SEQ ID NO: 39 - RNA, 110 nt, Homo sapiens
ggccuggcuggacagaguugucaugugucugccugucuacacuugcugugcagaacauccgcucaccuguacagcaggcacagacaggcagucacaugacaacccagccu

SEQ ID NO: 40 - RNA, 80 nt, Homo sapiens
ugggaugagguaguagguuguauaguuuuagggucacacccaccacugggagauaacuauacaaucuacugucuuuccua

SEQ ID NO: 41 - RNA, 80 nt, Homo sapiens
aggacccuuccagagggcccccccucaauccuguugugccuaauucagaggguuggguggaggcucuccugaagggcucu

SEQ ID NO: 42 - RNA, 110 nt, Homo sapiens
gcugcuggaagguguagguacccucaauggcucaguagccaguguagauccugucuuucguaaucagcagcuacaucuggcuacugggucucugauggcaucuucuagcu

SEQ ID NO: 43 - RNA, 83 nt, Homo sapiens
cggggugagguaguagguugugugguuucagggcagugauguugccccucggaagauaacuauacaaccuacugccuucccug

SEQ ID NO: 44 - RNA, 110 nt, Homo sapiens
aggaagcuucuggagauccugcuccgucgccccaguguucagacuaccuguucaggacaaugccguuguacaguagucugcacauugguuagacugggcaagggagagca

SEQ ID NO: 45 - RNA, 72 nt, Homo sapiens
ugucggguagcuuaucagacugauguugacuguugaaucucauggcaacaccagucgaugggcugucugaca

SEQ ID NO: 46 - RNA, 75 nt, Homo sapiens
agacagagaagccaggucacgucucugcaguuacacagcucacgagugccugcugggguggaaccuggucugucu

SEQ ID NO: 47 - RNA, 110 nt, Homo sapiens
aaagauccucagacaauccaugugcuucucuuguccuucauuccaccggagucugucucauacccaaccagauuucaguggagugaaguucaggaggcauggagcugaca

SEQ ID NO: 48 - RNA, 89 nt, Homo sapiens
accagacuuuuccuagucccugagacccuaacuugugagguauuuuaguaacaucacaagucaggcucuugggaccuaggcggagggga

SEQ ID NO: 49 - RNA, 72 nt, Homo sapiens
agguugagguaguagguuguauaguuuagaauuacaucaagggagauaacuguacagccuccuagcuuuccu

SEQ ID NO: 50 - RNA, 73 nt, Homo sapiens
ggccggcugggguuccuggggaugggauuugcuuccugucacaaaucacauugccagggauuuccaaccgacc

SEQ ID NO: 51 - RNA, 95 nt, Homo sapiens
ccagcucgggcagccguggccaucuuacugggcagcauuggauggagucaggucucuaauacugccugguaaugaugacggcggagcccugcacg

SEQ ID NO: 52 - RNA, 111 nt, Homo sapiens
agaguguucaaggacagcaagaaaaaugagggacuuucaggggcagcuguguuuucugacucagucauaaugccccuaaaaauccuuauuguucuugcagugugcaucggg

SEQ ID NO: 53 - RNA, 84 nt, Homo sapiens
aggcugagguaguaguuuguacaguuugagggucuaugauaccacccgguacaggagauaacuguacaggccacugccuugcca

SEQ ID NO: 54 - RNA, 78 nt, Homo sapiens
cugaggagcagggcuuagcugcuugugagcaggguccacaccaagucguguucacaguggcuaaguuccgccccccag

SEQ ID NO: 55 - RNA, 87 nt, Homo sapiens
agcuucccuggcucuagcagcacagaaauauuggcacagggaagcgagucugccaauauuggcugugcugcuccaggcaggguggug

SEQ ID NO: 56 - RNA, 83 nt, Homo sapiens
ucaggcugugacccucuugagggaagcacuuucuguugucugaaagaagagaaagugcuuccuuuuagaggcuuacugucuga

SEQ ID NO: 57 - RNA, 97 nt, Homo sapiens
accucucuaacaaggugcagagcuuagcugauuggugaacagugauugguuuccgcuuuguucacaguggcuaaguucugcaccugaagagaaggug

SEQ ID NO: 58 - RNA, 110 nt, Homo sapiens
ugaacauccaggucuggggcaugaaccuggcauacaauguagauuucuguguucguuaggcaacagcuacauugucugcuggguuucaggcuaccuggaaacauguucuc

SEQ ID NO: 59 - RNA, 72 nt, Homo sapiens
agauacuguaaacauccuacacucucagcuguggaaaguaagaaagcugggagaaggcuguuuacucuuucu

SEQ ID NO: 60 - RNA, 87 nt, Homo sapiens
accgcagggaaaaugagggacuuuugggggcagauguguuuccauuccacuaucauaaugccccuaaaaauccuuauugcucuugca

SEQ ID NO: 61 - RNA, 89 nt, Homo sapiens
accaugcuguaguguguguaaacauccuacacucucagcugugagcucaagguggcugggagaggguuguuuacuccuucugccaugga

SEQ ID NO: 62 - RNA, 129 nt, Homo sapiens
gggaugccacauucagccauucagcguacagugccuuucacagggaggugucauuuaugugaacuaaaauauaaauuucaccuuucugagaaggguaauguacagcaugcacugcauaugugguguccc

SEQ ID NO: 63 - RNA, 110 nt, Homo sapiens
cgccucagagccgcccgccguuccuuuuuccuaugcauauacuucuuugaggaucuggccuaaagagguauagggcaugggaaaacggggcggucggguccuccccagcg

SEQ ID NO: 64 - RNA, 82 nt, Homo sapiens
agggggcgagggauuggagagaaaggcaguuccugaugguccccuccccaggggcuggcuuuccucugguccuucccuccca

SEQ ID NO: 65 - RNA, 116 nt, Homo sapiens
caacuacagccacuacuacaggaccaucgaggaccugcgggacaagauucuuggugccaccauugagaacgccaggauuguccugcagaucaacaaugcucaacuggcugcagaug

SEQ ID NO: 66 - RNA, 69 nt, Homo sapiens
gggauacucaaaaugggggcgcuuuccuuuuugucuguacugggaagugcuucgauuuugggguguccc

SEQ ID NO: 67 - RNA, 87 nt, Homo sapiens
ucagagugagguaguagauuguauaguugugggguagugauuuuacccuguucaggagauaacuauacaaucuauugccuucccuga

SEQ ID NO: 68 - RNA, 76 nt, Homo sapiens
gagggggaagacgggaggaaagaagggagugguuccaucacgccuccucacuccucuccucccgucuucuccucuc

SEQ ID NO: 69 - RNA, 85 nt, Homo sapiens
cucaagcugugacucuccagagggaugcacuuucucuuaugugaaaaaaaagaaggcgcuucccuuuagagcguuacgguuuggg

SEQ ID NO: 70 - RNA, 127 nt, Homo sapiens
ggaugccacauucagccauucagugugcagugccuuucacagggaggugucauuuaugugaacuaaaauauaaauuucaccuuucugagaaggguaauguacagcaugcacugcauauguggugucc

SEQ ID NO: 71 - RNA, 97 nt, Homo sapiens
cacucugcuguggccuauggcuuuucauuccuaugugauugcugucccaaacucauguagggcuaaaagccaugggcuacagugaggggcgagcucc

SEQ ID NO: 72 - RNA, 72 nt, Homo sapiens
cuugggaauggcaaggaaaccguuaccauuacugaguuuaguaaugguaaugguucucuugcuauacccaga

SEQ ID NO: 73 - RNA, 88 nt, Homo sapiens
aucucuuacacaggcugaccgauuucuccugguguucagagucuguuuuugucuagcaccauuugaaaucgguuaugauguaggggga

SEQ ID NO: 74 - RNA, 70 nt, Homo sapiens
guuguuguaaacauccccgacuggaagcuguaagacacagcuaagcuuucagucagauguuugcugcuac

SEQ ID NO: 75 - RNA, 83 nt, Homo sapiens
ccuuggaguaaaguagcagcacauaaugguuuguggauuuugaaaaggugcaggccauauugugcugccucaaaaauacaagg

SEQ ID NO: 76 - RNA, 110 nt, Homo sapiens
ccagaggacaccuccacuccgucuacccaguguuuagacuaucuguucaggacucccaaauuguacaguagucugcacauugguuaggcugggcuggguuagacccucgg

SEQ ID NO: 77 - RNA, 68 nt, Homo sapiens
cccucgucuuacccagcaguguuugggugcgguugggagucucuaauacugccggguaaugauggagg

SEQ ID NO: 78 - RNA, 85 nt, Homo sapiens
cgcuggcgacgggacauuauuacuuuugguacgcgcugugacacuucaaacucguaccgugaguaauaaugcgccguccacggca

SEQ ID NO: 79 - RNA, 94 nt, Homo sapiens
cuuuggcgaucacugccucucugggccugugucuuaggcucugcaagaucaaccgagcaaagcacacggccugcagagaggcagcgcucugccc

SEQ ID NO: 80 - RNA, 90 nt, Homo sapiens
uuuccugcccucgaggagcucacagucuaguaugucucauccccuacuagacugaagcuccuugaggacagggauggucauacucaccuc

SEQ ID NO: 81 - RNA, 82 nt, Mus musculus
cagccugugauacucaaacugggggcucuuuuggauuuucaucggaagaaaagugccgccagguuuugagugucaccgguug

SEQ ID NO: 82 - RNA, 79 nt, Mus musculus
aaggcaggggugaggggcugcgggaggagccgggcggaggcugcggcuugcgcuucuccuggcucuccucccucucuuu

SEQ ID NO: 83 - RNA, 82 nt, Mus musculus
ccuauguagcggccaucaaaguggaggcccucucuugagccugaaugagaaagugcuuccacuuugugugccacugcauggg

SEQ ID NO: 84 - RNA, 83 nt, Mus musculus
cucaucuugcgguacucaaacuaugggggcacuuuuuuuuuucuuuaaaaagugccgccuaguuuuaagccccgccgguugag

SEQ ID NO: 85 - RNA, 70 nt, Mus musculus
ccugccagugguuuuacccuaugguagguuacgucaugcuguucuaccacaggguagaaccacggacagg

SEQ ID NO: 86 - RNA, 110 nt, Mus musculus
ccagaggauaccuccacuccgucuacccaguguuuagacuaccuguucaggacucccaaauuguacaguagucugcacauugguuaggcugggcuggguuagacccucgg

SEQ ID NO: 87 - RNA, 99 nt, Mus musculus
cauggcaccuccguuucccugaggagcccuuugagccuggagugaaaaaaaaaaacaggucaagaggcgccugggaacuggagaagaguguuaaacuuc

SEQ ID NO: 88 - RNA, 96 nt, Mus musculus
aaaaugaugaugucaguuggccggucggccgaucgcucggucugucagucagucggucggucgaucggucggucggucagucggcuuccugucuuc

SEQ ID NO: 89 - RNA, 82 nt, Mus musculus
ccaggccuuuggcagaggagggcuguucuucccuugaguuuuaugacugggaggaacuagccuucucucagcuuaggagugg

SEQ ID NO: 90 - RNA, 97 nt, Rattus norvegicus
agcgcuuuccugcccucgaggagcucacagucuaguaugucuccucccuacuagacugaggcuccuugaggacagggaucgucauacucaccucccg

SEQ ID NO: 91 - RNA, 92 nt, Rattus norvegicus
gagccccugcuggugggcgcggggcggggguuucagguggucucgcgguggccgcccgacugucccucugggucgcccagcuggggaguucc

SEQ ID NO: 92 - RNA, 101 nt, Homo sapiens
gcgagaagaucucaugcugugacucucuggagggaagcacuuucuguugucugaaagaaaacaaagcgcuucucuuuagaguguuacgguuugagaaaagc

SEQ ID NO: 93 - RNA, 71 nt, Homo sapiens
ugcccuagcagcgggaacaguucugcagugagcgaucggugcucugggguauuguuuccgcugccagggua

SEQ ID NO: 94 - RNA, 71 nt, Homo sapiens; identical to SEQ ID NO: 8

SEQ ID NO: 95 - RNA, 82 nt, Mus musculus; identical to SEQ ID NO: 81

SEQ ID NO: 96 - RNA, 98 nt, Mus musculus
gacccuuuggcgaucucugccucucugggccugugucuuaggcucuucaagauccaacgagcaaagcacagggccugcagagagguagcgcucugcuc

SEQ ID NO: 97 - RNA, 68 nt, Mus musculus
ccugcccucgaggagcucacagucuaguaugucuccucccuacuagacugaggcuccuugaggacagg

SEQ ID NO: 98 - RNA, 77 nt, Homo sapiens
guggccucguucaaguaauccaggauaggcugugcaggucccaaugggccuauucuugguuacuugcacggggacgc

SEQ ID NO: 99 - RNA, 89 nt, Homo sapiens
ugcugcuggccagagcucuuuucacauugugcuacugucugcaccugucacuagcagugcaauguuaaaagggcauuggccguguagug

SEQ ID NO: 100 - RNA, 110 nt, Homo sapiens
gaucugucugucuucuguauauacccuguagauccgaauuuguguaaggaauuuuguggucacaaauucguaucuaggggaauauguaguugacauaaacacuccgcucu

SEQ ID NO: 101 - RNA, 110 nt, Homo sapiens
ccagagguuguaacguugucuauauauacccuguagaaccgaauuugugugguauccguauagucacagauucgauucuaggggaauauauggucgaugcaaaaacuuca

SEQ ID NO: 102 - RNA, 88 nt, Homo sapiens
accaaguuucaguucauguaaacauccuacacucagcuguaauacauggauuggcugggagguggauguuuacuucagcugacuugga

SEQ ID NO: 103 - RNA, 92 nt, Homo sapiens
cggcuggacagcgggcaacggaaucccaaaagcagcuguugucuccagagcauuccagcugcgcuuggauuucguccccugcucuccugccu

SEQ ID NO: 104 - RNA, 96 nt, Homo sapiens
ucccuggcgugaggguaugugccuuuggacuacaucguggaagccagcaccaugcaguccaugggcauauacacuugccucaaggccuaugucauc

SEQ ID NO: 105 - RNA, 81 nt, Homo sapiens
cccauuggcauaaacccguagauccgaucuuguggugaaguggaccgcacaagcucgcuucuaugggucugugucagugug

SEQ ID NO: 106 - RNA, 84 nt, Homo sapiens
cuccccauggcccugucucccaacccuuguaccagugcugggcucagacccugguacaggccugggggacagggaccuggggac

SEQ ID NO: 107 - RNA, 64 nt, Homo sapiens
augacugauuucuuuugguguucagagucaauauaauuuucuagcaccaucugaaaucgguuau

SEQ ID NO: 108 - RNA, 85 nt, Homo sapiens
gcuaagcacuuacaacuguuugcagaggaaacugagacuuuguaacuaugucucagucucaucugcaaagaaguaagugcuuugc

SEQ ID NO: 109 - RNA, 97 nt, Rattus norvegicus
uguuggccuaguucuguguggaagacuagugauuuuguuguuuuuagauaacuaagacgacaacaaaucacagucugccauauggcacaggccaccu

SEQ ID NO: 110 - RNA, 95 nt, Rattus norvegicus
ggacagaccagcccugucuggaagacuagugauuuuguuguugugucuguguccaacaacaagucccagucugccacaugguguuggucacauca

SEQ ID NO: 111 - RNA, 24 nt, Homo sapiens
uucuccaaaagaaagcacuuucug

SEQ ID NO: 112 - RNA, 23 nt, Homo sapiens
caucuggagguaagaagcacuuu

SEQ ID NO: 113 - RNA, 107 nt, Homo sapiens
gugcucgcuucggcagcacauauacuaaaauuggaacgauacagagaagauuagcauggccccugcgcaaggaugacacgcaaauucgugaagcguuccauauuuuu

SEQ ID NO: 114 - DNA, 23 bp, Homo sapiens
tccataaagtaggaaacactaca

SEQ ID NO: 115 - DNA, 18 bp, Homo sapiens
ctcagtaatggtaacggt

SEQ ID NO: 116 - DNA, 20 bp, Homo sapiens
aaactcagtaatggtaacgg

SEQ ID NO: 117 - DNA, 23 bp, Homo sapiens
tccatcatcaaaacaaatggagt

SEQ ID NO: 118 - DNA, 17 bp, Homo sapiens
gggactttgtaggccag

SEQ ID NO: 119 - DNA, 23 bp, Homo sapiens
gaacaggtagtctgaacactggg

SEQ ID NO: 120 - DNA, 18 bp, Homo sapiens
tctgtatcgttccaattt

SEQ ID NO: 121 - DNA, 16 bp, Homo sapiens
gcgtgtcatccttgcg

SEQ ID NO: 122 - DNA, 18 bp, Homo sapiens
gaatcttgtcccgcaggt

SEQ ID NO: 123 - DNA, 21 bp, Mus musculus
gaacaggtagtctaaacactg

SEQ ID NO: 124 - DNA, 18 bp, Homo sapiens
ggactttgagggccagtt

SEQ ID NO: 125 - DNA, 22 bp, Homo sapiens
aaccaatgtgcagactactgta

SEQ ID NO: 126 - DNA, 16 bp, Mus musculus
gggcctccactttgat

SEQ ID NO: 127 - DNA, 21 bp, Homo sapiens
ataaggatttttaggggcatt

SEQ ID NO: 128 - DNA, 22 bp, Homo sapiens
cacaaaccattatgtgctgcta

SEQ ID NO: 129 - DNA, 13 bp, RATTUS NORVEGICUS
ggcgacccagagg

SEQ ID NO: 130 - DNA, 22 bp, Homo sapiens
acagttcttcaactggcagctt

SEQ ID NO: 131 - DNA, 21 bp, Homo sapiens
ctaccatagggtaaaaccact

SEQ ID NO: 132 - DNA, 17 bp, Homo sapiens
agtgcttccctccagag

SEQ ID NO: 133 - DNA, 23 bp, Homo sapiens
aacaaccagctaagacactgcca

SEQ ID NO: 134 - DNA, 22 bp, Homo sapiens
tgtaaaccatgatgtgctgcta

SEQ ID NO: 135 - DNA, 21 bp, Homo sapiens
ccaggttccaccccagcaggc

SEQ ID NO: 136 - DNA, 21 bp, Homo sapiens
ctgcctgtctgtgcctgctgt

SEQ ID NO: 137 - DNA, 18 bp, Homo sapiens
aaagtgcatccctctgga

SEQ ID NO: 138 - DNA, 23 bp, Homo sapiens
acaccccaaaatcgaagcacttc

SEQ ID NO: 139 - DNA, 22 bp, Homo sapiens
acaaagttctgtgatgcactga

SEQ ID NO: 140 - DNA, 18 bp, Homo sapiens
gaactgcctttctctcca

SEQ ID NO: 141 - DNA, 19 bp, Homo sapiens
agtgcttcttacctccaga

SEQ ID NO: 142 - DNA, 19 bp, Mus musculus
aagtgcccccatagtttga

SEQ ID NO: 143 - DNA, 17 bp, Homo sapiens
aactgttcccgctgcta

SEQ ID NO: 144 - DNA, 21 bp, Homo sapiens
gcggaacttagccactgtgaa

SEQ ID NO: 145 - DNA, 21 bp, Homo sapiens
ggggtatttgacaaactgaca

SEQ ID NO: 146 - DNA, 17 bp, RATTUS NORVEGICUS
tggcagactgtgatttg

SEQ ID NO: 147 - DNA, 24 bp, Homo sapiens
gagacccagtagccagatgtagct

SEQ ID NO: 148 - DNA, 22 bp, Homo sapiens
cttccagtcgaggatgtttaca

SEQ ID NO: 149 - DNA, 17 bp, Mus musculus
caaaagagcccccagtt

SEQ ID NO: 150 - DNA, 20 bp, Homo sapiens
tccagtcaaggatgtttaca

SEQ ID NO: 151 - DNA, 19 bp, RATTUS NORVEGICUS
actagactgtgagctcctc

SEQ ID NO: 152 - DNA, 17 bp, Mus musculus
ctcaaagggctcctcag

SEQ ID NO: 153 - DNA, 22 bp, Homo sapiens
acaaagttctgtgatgcactga

SEQ ID NO: 154 - DNA, 15 bp, Mus musculus
ggagagccaggagaa

SEQ ID NO: 155 - DNA, 24 bp, Homo sapiens
gacgggtgcgatttctgtgtgaga

SEQ ID NO: 156 - DNA, 21 bp, Homo sapiens
gccaatatttctgtgctgcta

SEQ ID NO: 157 - DNA, 21 bp, Homo sapiens
gcagaacttagccactgtgaa

SEQ ID NO: 158 - DNA, 20 bp, Homo sapiens
ctggaggaagggcccagagg

SEQ ID NO: 159 - DNA, 15 bp, Mus musculus
accgaccgaccgatc

SEQ ID NO: 160 - DNA, 22 bp, Homo sapiens
agcctatggaattcagttctca

SEQ ID NO: 161 - DNA, 15 bp, Mus musculus
ggccctgtgctttgc

SEQ ID NO: 162 - DNA, 16 bp, Mus musculus
ggagcctcagtctagt

SEQ ID NO: 163 - DNA, 17 bp, Mus musculus
tccgtggttctaccctg

SEQ ID NO: 164 - DNA, 21 bp, Homo sapiens
gccaatatttctgtgctgcta

SEQ ID NO: 165 - DNA, 21 bp, Homo sapiens
actgtacaaactactacctca

SEQ ID NO: 166 - DNA, 23 bp, Homo sapiens
gaaacccagcagacaatgtagct

SEQ ID NO: 167 - DNA, 15 bp, Homo sapiens
aagacgggaggagag

SEQ ID NO: 168 - DNA, 23 bp, Homo sapiens
gctgagagtgtaggatgtttaca

SEQ ID NO: 169 - DNA, 20 bp, Homo sapiens
accgatttcaaatggtgcta

SEQ ID NO: 170 - DNA, 21 bp, Homo sapiens
acaggattgagggggggccct

SEQ ID NO: 171 - DNA, 21 bp, Homo sapiens
actatacaacctcctacctca

SEQ ID NO: 172 - DNA, 22 bp, Homo sapiens
aactatacaatctactacctca

SEQ ID NO: 173 - DNA, 18 bp, Mus musculus
aagaacagccctcctctg

SEQ ID NO: 174 - DNA, 23 bp, Homo sapiens
gaacagatagtctaaacactggg

SEQ ID NO: 175 - DNA, 22 bp, Homo sapiens
tcaacatcagtctgataagcta

SEQ ID NO: 176 - DNA, 20 bp, Homo sapiens
ttttcccatgccctatacct

SEQ ID NO: 177 - DNA, 21 bp, Homo sapiens
gcaagcccagaccgcaaaaag

SEQ ID NO: 178 - DNA, 18 bp, Homo sapiens
aatgacacctccctgtga

SEQ ID NO: 179 - DNA, 18 bp, Homo sapiens
agaggtttcccgtgtatg

SEQ ID NO: 180 - DNA, 21 bp, Homo sapiens
gcattattactcacggtacga

SEQ ID NO: 181 - DNA, 21 bp, Homo sapiens
acagcacaaactactacctca

SEQ ID NO: 182 - DNA, 21 bp, Homo sapiens
ggaaatccctggcaatgtgat

SEQ ID NO: 183 - DNA, 16 bp, Homo sapiens
gaaaaacgccccctgg

SEQ ID NO: 184 - DNA, 22 bp, Homo sapiens
ctgttcctgctgaactgagcca

SEQ ID NO: 185 - DNA, 20 bp, Homo sapiens
ccaatatttacgtgctgcta

SEQ ID NO: 186 - DNA, 23 bp, Homo sapiens
ttcgccctctcaacccagctttt

SEQ ID NO: 187 - DNA, 22 bp, Homo sapiens
cagactccggtggaatgaagga

SEQ ID NO: 188 - DNA, 22 bp, Homo sapiens
ccatcattacccggcagtatta

SEQ ID NO: 189 - DNA, 23 bp, Homo sapiens
catcattaccaggcagtattaga

SEQ ID NO: 190 - DNA, 22 bp, Homo sapiens
cacaagttcggatctacgggtt

SEQ ID NO: 191 - DNA, 22 bp, Homo sapiens
aaccatacaacctactacctca

SEQ ID NO: 192 - DNA, 22 bp, Homo sapiens
aaccacacaacctactacctca

SEQ ID NO: 193 - DNA, 21 bp, Homo sapiens
gcctatcctggattacttgaa

SEQ ID NO: 194 - DNA, 22 bp, Homo sapiens
atgcccttttaacattgcactg

SEQ ID NO: 195 - DNA, 22 bp, Homo sapiens
aacctatcctgaattacttgaa

SEQ ID NO: 196 - DNA, 21 bp, Homo sapiens
gccaatatttctgtgctgcta

SEQ ID NO: 197 - DNA, 23 bp, Homo sapiens
cacaaattcggatctacagggta

SEQ ID NO: 198 - DNA, 20 bp, Homo sapiens
ctggaggaagggcccagagg

SEQ ID NO: 199 - DNA, 22 bp, Homo sapiens
acaaattcggttctacagggta

SEQ ID NO: 200 - DNA, 22 bp, Homo sapiens
ccatctttaccagacagtgtta

SEQ ID NO: 201 - DNA, 22 bp, Homo sapiens
agctgagtgtaggatgtttaca

SEQ ID NO: 202 - DNA, 22 bp, Homo sapiens
agctgcttttgggattccgttg

SEQ ID NO: 203 - DNA, 21 bp, Homo sapiens
gccaatatttctgtgctgcta

SEQ ID NO: 204 - DNA, 21 bp, Homo sapiens
actgtacaaactactacctca

SEQ ID NO: 205 - DNA, 16 bp, Homo sapiens
gaaaaacgccccctgg

SEQ ID NO: 206 - DNA, 18 bp, Homo sapiens
aagtgcttccctcaagag

SEQ ID NO: 207 - DNA, 22 bp, Homo sapiens
cacaagatcggatctacgggtt

SEQ ID NO: 208 - DNA, 18 bp, Homo sapiens
agtgctttcttttggaga

SEQ ID NO: 209 - DNA, 22 bp, Homo sapiens
agctgcttttgggattccgttg

SEQ ID NO: 210 - DNA, 24 bp, Homo sapiens
atccaatcagttcctgatgcagta

SEQ ID NO: 211 - DNA, 22 bp, Homo sapiens
cactggtacaagggttgggaga

SEQ ID NO: 212 - DNA, 21 bp, Homo sapiens
aaccgatttcagatggtgcta

SEQ ID NO: 213 - DNA, 19 bp, Homo sapiens
tctttgcagatgagactga

SEQ ID NO: 214 - DNA, 23 bp, Homo sapiens
ttcgccctctcaacccagctttt

SEQ ID NO: 215 - DNA, 22 bp, Homo sapiens
aactatacaacctactacctca

SEQ ID NO: 216 - DNA, 22 bp, Homo sapiens
tcacaagttagggtctcaggga

SEQ ID NO: 217 - DNA, 21 bp, Homo sapiens
tagctggttgaaggggaccaa

SEQ ID NO: 218 - DNA, 15 bp, RATTUS NORVEGICUS
actcagaagggtgcc

SEQ ID NO: 219 - RNA, 75 nt, Homo sapiens
ugcccuggcucaguuaucacagugcugaugcugucuauucuaaagguacaguacugugauaacugaaggauggca

SEQ ID NO: 220 - RNA, 88 nt, Homo sapiens
caccuuguccucacgguccaguuuucccaggaaucccuuagaugcuaagauggggauuccuggaaauacuguucuugaggucaugguu

SEQ ID NO: 221 - RNA, 89 nt, Homo sapiens
cgggguugguuguuaucuuugguuaucuagcuguaugagugguguggagucuucauaaagcuagauaaccgaaaguaaaaauaacccca

SEQ ID NO: 222 - RNA, 85 nt, Homo sapiens
ccuuagcagagcuguggagugugacaaugguguuugugucuaaacuaucaaacgccauuaucacacuaaauagcuacugcuaggc

SEQ ID NO: 223 - RNA, 84 nt, Homo sapiens
ugugcagugggaaggggggccgauacacuguacgagagugaguagcaggucucacagugaaccggucucuuucccuacuguguc

SEQ ID NO: 224 - RNA, 89 nt, Homo sapiens
gccggcgcccgagcucuggcuccgugucuucacucccgugcuuguccgaggagggagggagggacgggggcugugcuggggcagcugga

SEQ ID NO: 225 - RNA, 86 nt, Homo sapiens
ugccagucucuaggucccugagacccuuuaaccugugaggacauccagggucacaggugagguucuugggagccuggcgucuggcc

SEQ ID NO: 226 - RNA, 106 nt, Homo sapiens
gcgcagcgcccugucucccagccugaggugcagugcugcaucucuggucaguugggagucugagaugaagcacuguagcucaggaagagagaaguuguucugcagc

SEQ ID NO: 227 - RNA, 82 nt, Homo sapiens; identical to SEQ ID NO: 36

SEQ ID NO: 228 - DNA, 20 bp, Homo sapiens
cttcagttatcacagtactg

SEQ ID NO: 229 - DNA, 16 bp, Homo sapiens
tcctgggaaaactgga

SEQ ID NO: 230 - DNA, 22 bp, Homo sapiens
catacagctagataaccaaaga

SEQ ID NO: 231 - DNA, 18 bp, Homo sapiens
caccattgtcacactcca

SEQ ID NO: 232 - DNA, 19 bp, Homo sapiens
gaaagagaccggttcactg

SEQ ID NO: 233 - DNA, 16 bp, Homo sapiens
agtgaagacacggagc

SEQ ID NO: 234 - DNA, 19 bp, Homo sapiens
acaggttaaagggtctcag

SEQ ID NO: 235 - DNA, 20 bp, Homo sapiens
agctacagtgcttcatctca

SEQ ID NO: 236 - DNA, 21 bp, Homo sapiens
ccatcatcaaaacaaatggag

SEQ ID NO: 237 - RNA, 23 nt, Homo sapiens
uguaguguuuccuacuuuaugga

SEQ ID NO: 238 - RNA, 23 nt, Homo sapiens
aaaccguuaccauuacugaguuu

SEQ ID NO: 239 - RNA, 23 nt, Homo sapiens
aaaccguuaccauuacugaguuu

SEQ ID NO: 240 - RNA, 23 nt, Homo sapiens
acuccauuuguuuugaugaugga

SEQ ID NO: 241 - RNA, 21 nt, Homo sapiens
aacuggccuacaaagucccag

SEQ ID NO: 242 - RNA, 21 nt, Homo sapiens
uagguaguuucauguuguugg

SEQ ID NO: 243 - RNA, 23 nt, Homo sapiens
aggaccugcgggacaagauucuu

SEQ ID NO: 244 - RNA, 23 nt, Mus musculus
cccaguguuuagacuaucuguuc

SEQ ID NO: 245 - RNA, 24 nt, Homo sapiens
aacuggcccucaaagucccgcuuu

SEQ ID NO: 246 - RNA, 23 nt, Homo sapiens
cccaguguucagacuaccuguuc

SEQ ID NO: 247 - RNA, 22 nt, Mus musculus
caucaaaguggaggcccucucu

SEQ ID NO: 248 - RNA, 22 nt, Homo sapiens
uaaugccccuaaaaauccuuau

SEQ ID NO: 249 - RNA, 22 nt, Homo sapiens
uagcagcacauaaugguuugug

SEQ ID NO: 250 - RNA, 19 nt, Homo sapiens
ugucccucugggucgccca

SEQ ID NO: 251 - RNA, 22 nt, Homo sapiens
aagcugccaguugaagaacugu

SEQ ID NO: 252 - RNA, 21 nt, Homo sapiens
agugguuuuacccuaugguag

SEQ ID NO: 253 - RNA, 23 nt, Homo sapiens
ucucuggagggaagcacuuucug

SEQ ID NO: 254 - RNA, 23 nt, Homo sapiens
uggcagugucuuagcugguuguu

SEQ ID NO: 255 - RNA, 22 nt, Homo sapiens
uagcagcacaucaugguuuaca

SEQ ID NO: 256 - RNA, 21 nt, Homo sapiens
gccugcugggguggaaccugg

SEQ ID NO: 257 - RNA, 21 nt, Homo sapiens
acagcaggcacagacaggcag

SEQ ID NO: 258 - RNA, 21 nt, Homo sapiens
cuccagagggaugcacuuucu

SEQ ID NO: 259 - RNA, 23 nt, Homo sapiens
gaagugcuucgauuuuggggugu

SEQ ID NO: 260 - RNA, 22 nt, Homo sapiens
ucagugcaucacagaacuuugu

SEQ ID NO: 261 - RNA, 18 nt, Homo sapiens
uggagagaaaggcaguuc

SEQ ID NO: 262 - RNA, 23 nt, Homo sapiens
caucuggagguaagaagcacuuu

SEQ ID NO: 263 - RNA, 23 nt, Mus musculus
cucaaacuaugggggcacuuuuu

SEQ ID NO: 264 - RNA, 23 nt, Homo sapiens
uagcagcgggaacaguucugcag

SEQ ID NO: 265 - RNA, 21 nt, Homo sapiens
uucacaguggcuaaguuccgc

SEQ ID NO: 266 - RNA, 21 nt, Homo sapiens
ugucaguuugucaaauacccc

SEQ ID NO: 267 - RNA, 22 nt, Rattus norvegicus
caacaaaucacagucugccaua

SEQ ID NO: 268 - RNA, 24 nt, Homo sapiens
agcuacaucuggcuacugggucuc

SEQ ID NO: 269 - RNA, 22 nt, Homo sapiens
uguaaacauccucgacuggaag

SEQ ID NO: 270 - RNA, 22 nt, Mus musculus
acucaaacugggggcucuuuug

SEQ ID NO: 271 - RNA, 21 nt, Homo sapiens
uaaggcacccuucugaguaga

SEQ ID NO: 272 - RNA, 22 nt, Rattus norvegicus
ucgaggagcucacagucuagua

SEQ ID NO: 273 - RNA, 24 nt, Mus musculus
ucccugaggagcccuuugagccug

SEQ ID NO: 274 - RNA, 22 nt, Homo sapiens
ucagugcaucacagaacuuugu

SEQ ID NO: 275 - RNA, 23 nt, Mus musculus
gcuucuccuggcucuccucccuc

SEQ ID NO: 276 - RNA, 24 nt, Homo sapiens
ucucacacagaaaucgcacccguc

SEQ ID NO: 277 - RNA, 21 nt, Homo sapiens
uagcagcacagaaauauuggc

SEQ ID NO: 278 - RNA, 21 nt, Homo sapiens
uucacaguggcuaaguucugc

SEQ ID NO: 279 - RNA, 20 nt, Homo sapiens
ccucugggcccuuccuccag

SEQ ID NO: 280 - RNA, 21 nt, Mus musculus
ucgaucggucggucggucagu

SEQ ID NO: 281 - RNA, 22 nt, Homo sapiens
ugagaacugaauuccauaggcu

SEQ ID NO: 282 - RNA, 23 nt, Mus musculus
gcaaagcacagggccugcagaga

SEQ ID NO: 283 - RNA, 21 nt, Mus musculus
cuagacugaggcuccuugagg

SEQ ID NO: 284 - RNA, 22 nt, Mus musculus
uaccacaggguagaaccacgga

SEQ ID NO: 285 - RNA, 21 nt, Homo sapiens
uagcagcacagaaauauuggc

SEQ ID NO: 286 - RNA, 20 nt, Homo sapiens
gucaguuugucaaauacccc

SEQ ID NO: 287 - RNA, 23 nt, Homo sapiens
agcuacauugucugcuggguuuc

SEQ ID NO: 288 - RNA, 23 nt, Homo sapiens
ucacuccucuccucccgucuucu

SEQ ID NO: 289 - RNA, 23 nt, Homo sapiens
uguaaacauccuacacucucagc

SEQ ID NO: 290 - RNA, 20 nt, Homo sapiens
uagcaccauuugaaaucggu

SEQ ID NO: 291 - RNA, 21 nt, Homo sapiens
agggcccccccucaauccugu

SEQ ID NO: 292 - RNA, 21 nt, Homo sapiens
ugagguaggagguuguauagu

SEQ ID NO: 293 - RNA, 22 nt, Homo sapiens
ugagguaguagauuguauaguu

SEQ ID NO: 294 - RNA, 22 nt, mus musculus
ggcagaggagggcuguucuucc

SEQ ID NO: 295 - RNA, 23 nt, Homo sapiens
cccaguguuuagacuaucuguuc

SEQ ID NO: 296 - RNA, 22 nt, Homo sapiens
uagcuuaucagacugauguuga

SEQ ID NO: 297 - RNA, 22 nt, Homo sapiens
agagguauagggcaugggaaaa

SEQ ID NO: 298 - RNA, 21 nt, Homo sapiens
cuuuuugcggucugggcuugc

SEQ ID NO: 299 - RNA, 22 nt, Homo sapiens
uucacagggaggugucauuuau

SEQ ID NO: 300 - RNA, 24 nt, Homo sapiens
ugaaacauacacgggaaaccucuu

SEQ ID NO: 301 - RNA, 21 nt, Homo sapiens
ucguaccgugaguaauaaugc

SEQ ID NO: 302 - RNA, 21 nt, Homo sapiens
ugagguaguaguuugugcugu

SEQ ID NO: 303 - RNA, 21 nt, Homo sapiens
aucacauugccagggauuucc

SEQ ID NO: 304 - RNA, 23 nt, Homo sapiens
uuucaagccagggggcguuuuuc

SEQ ID NO: 305 - RNA, 22 nt, Homo sapiens
uggcucaguucagcaggaacag

SEQ ID NO: 306 - RNA, 22 nt, Homo sapiens
uagcagcacguaaauauuggcg

SEQ ID NO: 307 - RNA, 23 nt, Homo sapiens
aaaagcuggguugagagggcgaa

SEQ ID NO: 308 - RNA, 22 nt, Homo sapiens
uccuucauuccaccggagucug

SEQ ID NO: 309 - RNA, 22 nt, Homo sapiens
uaauacugccggguaaugaugg

SEQ ID NO: 310 - RNA, 23 nt, Homo sapiens
uaauacugccugguaaugaugac

SEQ ID NO: 311 - RNA, 22 nt, Homo sapiens
aacccguagauccgaacuugug

SEQ ID NO: 312 - RNA, 22 nt, Homo sapiens
ugagguaguagguuguaugguu

SEQ ID NO: 313 - RNA, 22 nt, Homo sapiens
ugagguaguagguugugugguu

SEQ ID NO: 314 - RNA, 21 nt, Homo sapiens
uucaaguaauccaggauaggc

SEQ ID NO: 315 - RNA, 22 nt, Homo sapiens
cagugcaauguuaaaagggcau

SEQ ID NO: 316 - RNA, 22 nt, Homo sapiens
uucaaguaauucaggauagguu

SEQ ID NO: 317 - RNA, 21 nt, Homo sapiens
uagcagcacagaaauauuggc

SEQ ID NO: 318 - RNA, 23 nt, Homo sapiens
uacccuguagauccgaauuugug

SEQ ID NO: 319 - RNA, 20 nt, Homo sapiens
ccucugggcccuuccuccag

SEQ ID NO: 320 - RNA, 22 nt, Homo sapiens
uacccuguagaaccgaauuugu

SEQ ID NO: 321 - RNA, 22 nt, Homo sapiens
uaacacugucugguaaagaugg

SEQ ID NO: 322 - RNA, 22 nt, Homo sapiens
uguaaacauccuacacucagcu

SEQ ID NO: 323 - RNA, 22 nt, Homo sapiens
caacggaaucccaaaagcagcu

SEQ ID NO: 324 - RNA, 21 nt, Homo sapiens
uagcagcacagaaauauuggc

SEQ ID NO: 325 - RNA, 21 nt, Homo sapiens
ugagguaguaguuuguacagu

SEQ ID NO: 326 - RNA, 22 nt, Homo sapiens
uaugugccuuuggacuacaucg

SEQ ID NO: 327 - RNA, 23 nt, Homo sapiens
cucuugagggaagcacuuucugu

SEQ ID NO: 328 - RNA, 22 nt, Homo sapiens
aacccguagauccgaucuugug

SEQ ID NO: 329 - RNA, 24 nt, Homo sapiens
uucuccaaaagaaagcacuuucug

SEQ ID NO: 330 - RNA, 22 nt, Homo sapiens
gcugcgcuuggauuucgucccc

SEQ ID NO: 331 - RNA, 24 nt, Homo sapiens
uacugcaucaggaacugauuggau

SEQ ID NO: 332 - RNA, 22 nt, Homo sapiens
ucucccaacccuuguaccagug

SEQ ID NO: 333 - RNA, 21 nt, Homo sapiens
uagcaccaucugaaaucgguu

SEQ ID NO: 334 - RNA, 22 nt, Homo sapiens
uguuugcagaggaaacugagac

SEQ ID NO: 335 - RNA, 23 nt, Homo sapiens
aaaagcuggguugagagggcgaa

SEQ ID NO: 336 - RNA, 22 nt, Homo sapiens
ugagguaguagguuguauaguu

SEQ ID NO: 337 - RNA, 22 nt, Homo sapiens
ucccugagacccuaacuuguga

SEQ ID NO: 338 - RNA, 21 nt, Homo sapiens
uugguccccuucaaccagcua

SEQ ID NO: 339 - RNA, 21 nt, Rattus norvegicus
uggaagacuagugauuuuguu

SEQ ID NO: 340 - RNA, 22 nt, Homo sapiens
uacaguacugugauaacugaag

SEQ ID NO: 341 - RNA, 24 nt, Homo sapiens
guccaguuuucccaggaaucccuu

SEQ ID NO: 342 - RNA, 23 nt, Homo sapiens
ucuuugguuaucuagcuguauga

SEQ ID NO: 343 - RNA, 23 nt, Homo sapiens
uggagugugacaaugguguuugu

SEQ ID NO: 344 - RNA, 20 nt, Homo sapiens
gucaguuugucaaauacccc

SEQ ID NO: 345 - RNA, 22 nt, Homo sapiens
ucuggcuccgugucuucacucc

SEQ ID NO: 346 - RNA, 23 nt, Homo sapiens
ucccugagacccuuuaaccugug

SEQ ID NO: 347 - RNA, 22 nt, Homo sapiens
ugagaugaagcacuguagcuca

SEQ ID NO: 348 - RNA, 23 nt, Homo sapiens
acuccauuuguuuugaugaugga

SEQ ID NO: 349 - RNA, 72 nt, Homo sapiens; identical to SEQ ID NO: 49

SEQ ID NO: 350 - RNA, 83 nt, Homo sapiens; identical to SEQ ID NO: 15

SEQ ID NO: 351 - RNA, 87 nt, Homo sapiens
ggaagcgaguuguuaucuuugguuaucuagcuguaugaguguauuggucuucauaaagcuagauaaccgaaaguaaaaacuccuuca

SEQ ID NO: 352 - RNA, 90 nt, Homo sapiens
ggaggcccguuucucucuuugguuaucuagcuguaugagugccacagagccgucauaaagcuagauaaccgaaaguagaaaugauucuca

SEQ ID NO: 353 - RNA, 81 nt, Homo sapiens; identical to SEQ ID NO: 10

SEQ ID NO: 354 - RNA, 73 nt, Homo sapiens; identical to SEQ ID NO: 27

SEQ ID NO: 355 - RNA, 84 nt, Homo sapiens
ggcuguggcuggauucaaguaauccaggauaggcuguuuccaucugugaggccuauucuugauuacuuguuucuggaggcagcu

SEQ ID NO: 356 - RNA, 72 nt, Homo sapiens; identical to SEQ ID NO: 59

SEQ ID NO: 357 - RNA, 79 nt, Homo sapiens
acuguccuuuuucgguuaucaugguaccgaugcuguauaucugaaagguacaguacugugauaacugaagaaugguggu

SEQ ID NO: 358 - RNA, 89 nt, Homo sapiens; identical to SEQ ID NO: 48

SEQ ID NO: 359 - RNA, 90 nt, Homo sapiens; identical to SEQ ID NO: 31

SEQ ID NO: 360 - RNA, 110 nt, Homo sapiens; identical to SEQ ID NO: 44

SEQ ID NO: 361 - RNA, 111 nt, Homo sapiens; identical to SEQ ID NO: 52

SEQ ID NO: 362 - RNA, 127 nt, Homo sapiens; identical to SEQ ID NO: 70

SEQ ID NO: 363 - RNA, 83 nt, Homo sapiens
ucucaugcagucauucuccaaaagaaagcacuuucuguugucugaaagcagagugccuucuuuuggagcguuacuguuugaga

SEQ ID NO: 364 - RNA, 90 nt, Homo sapiens
ucucagguugugaccuucucgaggaaagaagcacuuucuguugucugaaagaaaagaaagugcuuccuuucagaggguuacgguuugaga

SEQ ID NO: 365 - RNA, 85 nt, Homo sapiens
ucucaugaugugaccaucuggagguaagaagcacuuuguguuuugugaaagaaagugcuuccuuucagaggguuacucuuugaga

SEQ ID NO: 366 - RNA, 90 nt, Homo sapiens
ucucaggcugugaccaucuggagguaagaagcacuuucuguuuugugaaagaaaagaaagugcuuccuuucagaggguuacucuuugaga

SEQ ID NO: 367 - RNA, 95 nt, rattus norvegicus; identical to SEQ ID NO: 110

SEQ ID NO: 368 - DNA, 45 bp, Artificial sequence
  RT PCR Primer
acttttgagggggacacagaccttctaagttttgagatcaacatc

SEQ ID NO: 369 - DNA, 44 bp, Artificial sequence
  RT PCR Primer
acttttgagggggacacagaccttctaagttttgagaggaaatc

SEQ ID NO: 370 - DNA, 45 bp, Artificial sequence
  RT PCR Primer
acttttgagggggacacagaccttctaagttttgagagcggaact

SEQ ID NO: 371 - DNA, 45 bp, Artificial sequence
  RT PCR Primer
acttttgagggggacacagaccttctaagttttgagagcaactta

SEQ ID NO: 372 - DNA, 45 bp, Artificial sequence
  RT PCR Primer
acttttgagggggacacagaccttctaagttttgagatcacaagt

SEQ ID NO: 373 - DNA, 45 bp, Artificial sequence
  RT PCR Primer
acttttgagggggacacagaccttctaagttttgagatccatcat

SEQ ID NO: 374 - DNA, 45 bp, Artificial sequence
  RT PCR Primer
acttttgagggggacacagaccttctaagttttgagaaaccacac

SEQ ID NO: 375 - DNA, 45 bp, Artificial sequence
  RT PCR Primer
acttttgagggggacacagaccttctaagttttgagaaactatac

SEQ ID NO: 376 - DNA, 34 bp, Artificial sequence
  miR specific forward primer
aacctcagcctcgctatggttagcttatcagact

SEQ ID NO: 377 - DNA, 33 bp, Artificial sequence
  miR specific forward primer
aacctcagcctcgctatgggatcacattgccag

SEQ ID NO: 378 - DNA, 32 bp, Artificial sequence
  miR specific forward primer
aacctcagcctcgctatggttcacagtggcta

SEQ ID NO: 379 - DNA, 33 bp, Artificial sequence
  miR specific forward primer
aacctcagcctcgctatgggtattgcacattac

SEQ ID NO: 380 - DNA, 30 bp, Artificial sequence
  miR specific forward primer
aacctcagcctcgctatggtccctgagacc

SEQ ID NO: 381 - DNA, 33 bp, Artificial sequence
  miR specific forward primer
aacctcagcctcgctatgggactccatttgttt

SEQ ID NO: 382 - DNA, 33 bp, Artificial sequence
  miR specific forward primer
aacctcagcctcgctatgggtgaggtagtaggt

SEQ ID NO: 383 - DNA, 33 bp, Artificial sequence; identical to SEQ ID NO: 382
  miR specific forward primer

SEQ ID NO: 384 - DNA, 10 bp, Artificial sequence
  miR specific primer
agactgatgt

SEQ ID NO: 385 - DNA, 9 bp, Artificial sequence
  miR specific primer
cagggattt

SEQ ID NO: 386 - DNA, 11 bp, Artificial sequence
  miR specific probe
ctaagttccgc

SEQ ID NO: 387 - DNA, 11 bp, Artificial sequence
  miR specific probe
tactaagttgc

SEQ ID NO: 388 - DNA, 9 bp, Artificial sequence
  miR specific probe
aagttaggg

SEQ ID NO: 389 - DNA, 9 bp, Artificial sequence
  miR specific probe
tgatgatgg

SEQ ID NO: 390 - DNA, 10 bp, Artificial sequence
  miR specific probe
accacacaac

SEQ ID NO: 391 - DNA, 12 bp, Artificial sequence
  miR specific probe
actatacaacct

SEQ ID NO: 392 - DNA, 20 bp, Artificial sequence
  Target specific reverse primer
atcagcgatcttggtggttt

SEQ ID NO: 393 - DNA, 20 bp, Artificial sequence
  Target specific reverse primer
aggggccatgctaatcttct

SEQ ID NO: 394 - DNA, 25 bp, Artificial sequence
  Target specific forward primer
gcagatgatgtaaaagaatatttgc

SEQ ID NO: 395 - DNA, 22 bp, Artificial sequence
  Target specific forward primer
gcttcggcagcacatatactaa

SEQ ID NO: 396 - DNA, 10 bp, Artificial sequence
  miR specific probe
agagatggtg

SEQ ID NO: 397 - DNA, 9 bp, Artificial sequence
  miR specific probe
atcgttcca

SEQ ID NO: 398 - DNA, 19 bp, Artificial sequence
  Universal primers
aacctcagcctcgctatgg

SEQ ID NO: 399 - DNA, 20 bp, Artificial sequence
  Universal primers
acttttgagggggacacaga

SEQ ID NO: 400 - RNA, 24 nt, Homo sapiens
guccaguuuucccaggaaucccuu

SEQ ID NO: 401 - DNA, 45 bp, Artificial sequence
  RT_DNA_hsa-miR-145 (201)
acttttgagggggacacagaccttctaagttttgagaaagggatt

SEQ ID NO: 402 - DNA, 33 bp, Artificial sequence
  F_primer_hsa-miR-145 (201)
aacctcagcctcgctatggggtccagttttccc

SEQ ID NO: 403 - DNA, 19 bp, Artificial sequence
  FP_NM000201, Amp 1
aacctcagcctcgctatgg

SEQ ID NO: 404 - DNA, 20 bp, Artificial sequence
  RP_NM_000201, Amp 1
acttttgagggggacacaga

SEQ ID NO: 405 - DNA, 12 bp, Artificial sequence
  hsa-miR-145-qPCR-Probe1#Q2
ccaggaatccct